(19) United States
(12) Patent Application Publication
(10) Pub. No.: US 2026/0207623 A1
(43) Pub. Date: Jul. 23, 2026

(54) USE OF ESTETROL IN HEPATICALLY IMPAIRED PATIENTS

(71) Applicant: ESTETRA SRL, Liége (BE)

(72) Inventors: Mélanie Taziaux, Liège (BE); Anne Gallez, Liège (BE)

(73) Assignee: ESTETRA SRL, Liége (BE)

(21) Appl. No.: 19/139,201

(22) PCT Filed: Dec. 19, 2023
(86) PCT No.: PCT/EP2023/086561

§ 371 Date: Jun. 13, 2025

(30) Foreign Application Priority Data
Dec. 20, 2022 (EP) 22215129.2

(51) Int. Cl. A61K 31/565; A61K 31/55; A61K 31/567; A61K 31/57; A61K 31/585; A61P 5/30; A61P 15/12

(52) U.S. Cl. CPC A61K 31/565; A61K 31/55; A61K 31/567; A61K 31/57; A61K 31/585; A61P 5/30; A61P 15/12

(57) ABSTRACT

The present invention relates to a composition comprising an estetrol component for use in alleviating estrogen deficiency symptoms including menopause-associated symptoms in subjects that are characterized by a degree of hepatic impairment. The composition described herein displays favorable pharmacokinetic properties when compared to existing estrogen-based compositions that aim to alleviate estrogen deficiency symptoms. Also described herein are uses related to the above and corresponding methods of treatment.

### FIELD OF THE INVENTION

The present invention broadly relates to the field of medicine, and more particularly to hormone treatments of female menopausal subjects characterized by hepatic impairment. Specifically, the invention relates to a composition comprising an estetrol component for use in treating hepatically impaired subjects in a safe manner and wherein the composition is effective for estrogen deficiency symptoms in said subjects.

### BACKGROUND OF THE INVENTION

The liver is the main organ for phase I and phase II drug metabolic processes and is involved in the clearance of many drugs through a variety of oxidative and conjugative metabolic pathways and/or through biliary excretion of unchanged drugs or metabolites thereof (Almazroo et al., Clin Liver Dis, 2017).

Many reports in the biomedical literature have documented that hepatic diseases can alter the absorption and disposition of drugs (PK) as well as their efficacy and safety (PD) due to alterations of these excretory and metabolic activities. Such a hepatic impairment can lead to the accumulation of the drug and its metabolites and/or a failure to form an active metabolite, subsequently resulting in different sensitivities to pharmacological effects of the drug that is administered, both desired and adverse (Verbeeck, Eur J Clin Pharmacol, 2008; FDA report on pharmacokinetics in patients with impaired hepatic function, Clinical Pharmacology, 2003). These reports have been based on studies in patients with common hepatic diseases, such as alcoholic liver disease and chronic infections with hepatitis viruses B and C, and less common diseases, such as acute hepatitis D or E, primary biliary cirrhosis, primary sclerosing cholangitis, and alpha1-antitrypsin deficiency. The specific impact of any disease on hepatic function is often poorly described and highly variable, particularly with regard to effects on the PK and PD of a drug.

In an average ageing population, the use of drugs aiming to reduce one or more estrogen deficiency symptoms (such as menopause-associated symptoms) is widespread. Menopause has been described in detail and can be considered as a gradual process involving different hormonal changes over a period of typically multiple years resulting in a permanent arrest of ovarian follicular activity and menstrual cycles. Briefly, the early stages of menopause include increased levels of follicle stimulating hormone and a decline of inhibin B expression, albeit estrogen levels are generally preserved when compared to those typically observed in a pre-menopausal woman.

As time progresses, estrogen levels eventually decline as permanent cessation of menses ensues (Djahanbakhch et al., J Pathol, 2007). The menopausal process can impart a plethora of unwanted effects to a (peri)-menopausal woman, including but not limited to vasomotor symptoms (VMS) (e.g. night sweats, hot flashes, and flushes) (Utian et al., Menopause, 2005), and thinning of the epithelial linings of the vagina and urethra resulting in the genitourinary syndrome of menopause (GSM), which encompasses a wide array of symptoms such as vulvovaginal atrophy (VVA), urinary complaints, vaginal dryness, itching, dyspareunia, dysuria, increased urinary frequency and an increased risk of recurrent urinary infections (Portman et al., Menopause, 2014). Moreover, menopause may have a profound effect on the psychological state of the subject leading to for example depression, irritability, mood changes, insomnia, sleep disturbance, anxiety, and nervous tensions. These psychological aspects of menopause can arise independently of the physiological symptoms, but may equally be a consequence thereof. While considerable inter-person variability exists, the potential impact of menopause on the general well-being of a person should therefore not be underestimated or minimized.

To date, estrogen therapy (i.e. hormone replacement therapy) remains the gold standard for relief of menopausal symptoms, in particular VMS. Estrogen therapy is the most consistently effective treatment used in the US and Europe to treat VMS due to menopause. Following the safety issues reported in the primary Women's Health Initiative publications (Anderson et al., JAMA, 2004) and with continued subject requests for treatment, it has been a challenge for clinicians to identify the lowest effective dose of estrogen for alleviating menopausal symptoms especially in subjects that have a medical precondition (Simon et al., Expert Opin Investig Drugs, 2007). In addition, it remains a general challenge to develop safer estrogen-based therapy than those currently used.

As with any drugs in general, it cannot be predicted how estrogen will be metabolized by hepatically impaired subjects and therefore the efficacy, safety and tolerability properties of these drugs are unknown. Generally, drugs containing an estrogen have been explicitly contraindicated in subjects with hepatic impairment because of altered exposure to one or more of the pharmaceutically active ingredients or metabolites thereof. As an illustrative example, reference is made to Angeliq®, BIJUVA®, or DUAVEE®, where said contraindication is explicitly mentioned in the Food and Drug Administration (FDA)-approved patient label. Indeed, estrogens may be poorly metabolized in women with impaired liver function. Therefore, it is unknown if the use of respective drugs in these patients is safe or if adjustments are required.

The provision of any estrogen-based drug directed to alleviating estrogen deficiency symptoms that is safe to use in subjects with hepatic impairment is therefore of great interest to both subjects and healthcare professionals, and forms an unmet need to date.

As a precondition, there is a need to achieve favorable pharmacokinetic parameters despite the presence of a hepatic impairment in the subjects.

### SUMMARY OF THE INVENTION

The inventors have observed that estetrol is well-tolerated in hepatically impaired subjects and therefore provides a safer means to treat estrogen deficiency symptoms (such as menopause-associated symptoms) in hepatically impaired subjects when compared to presently available products (reference is made to the background section). More particularly, estetrol dosages from about 15 mg to 25 mg, and more particularly dosages from about 15 mg and about 20 mg of estetrol component were well tolerated in all tested subjects, and no significant treatment-related adverse events (TEAEs) were observed. All subjects maintained optimal vital signs, and laboratory values as further illustrated in the Examples section.

Indeed, the hormone replacement therapy of the invention (having the aim to alleviate menopause-associated symptoms, and more broadly estrogen deficiency symptoms) was found to be particularly beneficial and to have an extremely limited impact on a large number of hepatic, haemostatic, endocrine and metabolic parameters in hepatically impaired subjects. Surprisingly and very beneficially, the treatment of the present invention, in contrast, was found to not modify, or to only minimally modify, most parameters.

More particularly, the pharmacokinetic properties of a pharmaceutical composition comprising estetrol as the estrogenic component for use in alleviating estrogen deficiency symptoms were generally favourable for subjects having varying degrees of hepatic impairment when compared to compositions for the alleviation of estrogen deficiency symptoms such as hormone replacement therapies that are known to date. The favourable pharmacokinetic properties encompass parameters including the maximum observed plasma concentration (Cₘₐₓ) of the estetrol component, area under the concentration-time curve (AUC) from time zero extrapolated to infinity (AUC_{inf}) of the estetrol component, AUC_{inf} of E4-3-glucuronide, and half-life times of the estetrol component and its metabolites.

Based on the above pharmacokinetic findings, it was found to be surprisingly possible to treat estrogen-deficiency symptoms in patients with varying degrees of hepatic impairment with the same estetrol dose used in patients with normal hepatic function, without any significant difference in terms of safety and efficacy. The same effect on the endometrium is observed in patients with and without hepatic impairment: using a dose of 15 mg estetrol or more, fewer biopsies are required than with the lower dose of 10 mg in both populations. Based on these findings, it is now possible that patients with hepatic impairment can solely be treated with an estrogen—namely estetrol—against estrogen deficiency symptoms and especially menopausal symptoms, without a requirement to counteract the effect on the endometrium with a progestogen such as drospirenone. In addition, hysterectomized patients with impaired liver function also benefit from the respective therapy which has the advantage that the patients are not burdened with a progestogen that they do not need. Such a therapy, i.e. a therapy that is suitable for both hysterectomized and non-hysterectomized hepatically impaired patients, has not been described to date. The invention therefore provides the following aspects:
Aspect 1. A composition for use in alleviating estrogen deficiency symptoms in a hepatically impaired subject, wherein said composition comprises an estetrol component and wherein said composition is administered at a daily amount equivalent to from about 15 mg to about 25 mg of estetrol.
Aspect 2. Use of a composition for the manufacturing of a medicament for alleviating estrogen deficiency symptoms in a hepatically impaired subject, wherein said composition comprises an estetrol component and wherein said composition is administered at a daily amount equivalent to from about 15 mg to about 25 mg of estetrol.
Aspect 3. Use of a composition comprising an estetrol component for alleviating estrogen deficiency symptoms in a hepatically impaired subject, wherein said composition is administered at a daily amount equivalent to from about 15 mg to about 25 mg of estetrol.
Aspect 4. A method of alleviating (i.e. treating) estrogen deficiency symptoms in a hepatically impaired subject, wherein said method comprises the step of administering a composition comprising an estetrol component at a daily amount equivalent to from about 15 mg to about 25 mg of estetrol.
Aspect 5. A method to improve the safety of therapy for estrogen deficiency symptoms in a hepatically impaired subject, wherein said method comprises administration of a composition comprising an estetrol component at a daily amount equivalent to from about 15 mg to about 25 mg.
Aspect 6. The composition for use according to aspect 1, the use according to aspects 2 or 3, or the method according to aspects 4 or 5, wherein the hepatically impaired subject is characterised by a Child-Pugh score of at least 5 (corresponding to Grade A hepatic impairment according to the Child-Pugh score).
Aspect 7. The composition for use, the use, or the method according to any one of the preceding aspects, wherein the hepatically impaired subject is characterised by a Child-Pugh score of from 5 to 15 (corresponding to Grade A, B, or C hepatic impairment according to the Child-Pugh score).
Aspect 8. The composition for use, the use, or the method according to any one of the preceding aspects, wherein the hepatically impaired subject is characterised by a mild hepatic impairment (Child-Pugh score of 5 or 6 points, alternatively indicated as “Grade A”), a moderate hepatic impairment (Child-Pugh score of 7 to 9 points, alternatively indicated as “Grade B”), or a severe hepatic impairment (Child-Pugh score of 10 to 15 points, alternatively indicated as “Grade C”), as classified by the Child-Pugh scoring system.
Aspect 9. The composition for use, the use, or the method according to any one of the preceding aspects, wherein the hepatically impaired subject is characterised by a mild hepatic impairment (Grade A) or moderate hepatic impairment (Grade B) as classified by the Child-Pugh scoring system.
Aspect 10. The composition for use, the use, or the method according to any one of the preceding aspects, wherein the estrogen deficiency symptoms are menopause-associated symptoms.
Aspect 11. The composition for use, the use, or the method according to any one of the preceding aspects, wherein the estrogen deficiency symptoms are psychological aspects of menopausal transition.
Aspect 12. The composition for use, the use, or the method according to aspect 11, wherein the psychological aspects of menopausal transition are selected from the group consisting of: depression, irritability, mood changes, insomnia, sleep disturbance, anxiety, nervous tension and any combination thereof.
Aspect 13. The composition for use, the use, or the method according to any one of the preceding aspects, wherein the estrogen deficiency symptoms are physiological aspects of menopausal transition.
Aspect 14. The composition for use, the use, or the method according to aspect 13, wherein the physiological aspects of menopausal transition are selected from the group consisting of: joint pain, loss of bone density, urinary tract infections, urinary incontinence, dryness of the vagina, uterine prolapse, changes in skin texture, weight gain, dyspareunia, cardiovascular diseases, diabetes and any combination thereof.
Aspect 15. The composition for use, the use, or the method according to any one of the preceding aspects, for use in reducing VMS frequency, VMS severity, hot flush weekly weighted score, dryness of the vagina, dyspareunia, or any combination thereof, or for use in improving Quality of Life according to the Menopause Rating Scale (MRS) and/or the Menopause-specific Quality of Life (MENQOL) questionnaires.
Aspect 16. The composition for use, the use, or the method according to any one of the preceding aspects, wherein the composition is formulated as an oral, sublingual, buccal, or sublabial dosage unit, preferably wherein the composition is formulated as an oral dosage unit.
Aspect 17. The composition for use, the use, or the method according to aspect 16, wherein a single administration of the oral dosage unit provides a hepatically impaired subject with a pharmacokinetic profile characterized by a geometric mean (GM) Cmax for estetrol (E4) which is less than 2 fold the corresponding GM Cmax in a subject having a normal hepatic function, preferably wherein the geometric mean (GM) Cmax is about 1.7 fold or less of the corresponding GM Cmax in a subject having a normal hepatic function.
Aspect 18. The composition for use, the use, or the method according to aspect 16 or 17, wherein a single administration of the oral dosage unit provides a hepatically impaired subject with a pharmacokinetic profile characterized by a GM AUCinf for E4 of less than about 2 fold the corresponding GM AUCinf in a subject having a normal hepatic function, preferably wherein the GM AUCinf of E4 is about 1.1 fold or less of the corresponding GM AUCinf in a subject having a normal hepatic function.
Aspect 19. The composition for use, the use, or the method according to any one of aspects 16 to 18, wherein a single administration of the oral dosage unit provides a hepatically impaired subject with a pharmacokinetic profile characterized by a GM AUCinf for E4-3-glucuronide which does not significantly differ from the corresponding GM AUCinf of E4-3-glucuronide in a subject having a normal hepatic function.
Aspect 20. The composition for use, the use, or the method according to any one of aspects 16 to 19, wherein a single administration of the oral dosage unit provides a hepatically impaired subject with a pharmacokinetic profile characterized by a GM T½ for E4 which is similar when compared to the corresponding GM T½ in a subject having a normal hepatic function.
Aspect 21. The composition for use, the use, or the method according to any one of the preceding aspects, wherein the number, frequency, and/or severity of adverse effects do not differ between a population of hepatically impaired subjects and a population of subjects having a normal hepatic function.
Aspect 22. The composition for use, the use, or the method according to any one of the preceding aspects, wherein the subject is a female menopausal, perimenopausal, or post-menopausal subject.
Aspect 23. The composition for use, the use, or the method according to any one of the preceding aspects, wherein the composition comprises from about 15 mg to about 25 mg of estetrol component.
Aspect 24. The composition for use, the use, or the method according to any one of the preceding aspects, wherein the composition comprises from about 15 mg to about 20 mg of estetrol component.
Aspect 25. The composition for use, the use, or the method according to any one of the preceding aspects, wherein the composition comprises about 15 mg of estetrol component.
Aspect 26. The composition for use, the use, or the method according to any one of aspects 1 to 24, wherein the composition comprises about 20 mg of estetrol component.
Aspect 27. The composition for use, the use, or the method according to any one of the preceding aspects, wherein the estetrol component is estetrol or an ester thereof.
Aspect 28. The composition for use, the use, or the method according to any one of aspects 1 to 26, wherein the estetrol component is estetrol monohydrate.
Aspect 29. The composition for use, the use, or the method according to any one of the preceding aspects, wherein the composition further comprises a progestogenic component.
Aspect 30. The composition for use, the use, or the method according to aspect 29, wherein the progestogenic component is selected from the group consisting of: progesterone, drospirenone, norethisterone, norethisterone-acetate (NETA), dydrogesterone, levonorgestrel (LNG), etonogestrel, norgestrel, nomegestrol, nomegestrol-acetate (NOMAC), trimegestone, nestorone, dydrogesterone, gestodene, desogestrel, norgestimate, cyproterone acetate, dienogest, and chlormadinone.
Aspect 31. The composition for use, the use, or the method according to aspect 29 or 30, wherein said progestogenic component is selected from the group comprising drospirenone, progesterone, or dydrogesterone.
Aspect 32. The composition for use, the use, or the method according to any one of aspects 29 to 31, wherein the progestogenic component is drospirenone, preferably of from about 0.25 mg to about 10 mg of drospirenone, more preferably from about 1 mg to about 4 mg of drospirenone, more preferably about 3 mg of drospirenone, or wherein said progestogenic component is administered in an amount equivalent to from about 0.25 mg to about 10 mg of drospirenone, preferably in an amount equivalent to from about 1 mg to about 4 mg of drospirenone, more preferably in an amount equivalent to about 3 mg drospirenone.
Aspect 33. The composition for use, the use, or the method according to any one of aspects 29 to 32, wherein the progestogenic component is progesterone, preferably from about 25 mg to about 300 mg of progesterone, more preferably about 100 mg to about 200 mg of progesterone, or wherein said progestogenic component is administered in an amount equivalent to from about 25 mg to about 300 mg of progesterone, preferably in an amount equivalent to from about 100 mg to about 200 mg.
Aspect 34. The composition for use, the use, or the method according to any one of aspects 29 to 33, wherein the progestogenic component is dydrogesterone, preferably of from about 1 mg to about 20 mg dydrogesterone, more preferably from about 5 mg to about 10 mg dydrogesterone, or wherein said progestogenic component is administered in an amount equivalent to from about 1 mg to about 20 mg dydrogesterone, preferably in an amount equivalent to from about 5 mg to about 10 mg dydrogesterone.
Aspect 35. The composition for use, the use, or the method according to any one of aspects 1 to 28, wherein the composition further comprises bazedoxifene.
Aspect 36. The composition for use, the use, or the method according to any one of aspects 1 to 28, wherein the estetrol component is the single (i.e. only) pharmaceutically active ingredient in the composition.
Aspect 37. The composition for use, the use, or the method according to any one of the preceding aspects, wherein the composition is formulated to correspond to a daily dosage unit.
Aspect 38. The composition for use, the use, or the method according to any one of the preceding aspects, wherein the composition is used in a once-a-day multiple dose regimen.
Aspect 39. In any one of the aspects defined herein, said dosage unit may be presented as a kit-of-parts containing a packaging unit, e.g. a blister pack, containing the daily oral dosage units comprising the estetrol component. The skilled person will additionally know that, within the scope of the present invention, each packaging unit, e.g. blister pack, may be numbered or otherwise marked.

Within the scope of the invention, each such packaging unit may be a sealed blister pack with a cardboard, paperboard, foil plastic backing and enclosed in a suitable cover.

Also envisaged in any one of the aspects of such packaging units as defined herein are bottles. The material of the bottle is not particularly limiting. In preferred embodiments, the bottle is a glass bottle characterized by a color capable of reducing or preventing degradation of the contents of the bottle by e.g. UV light while maintaining a degree of transparency that allows for visual inspection of the contents of said bottle. Suitable colors include without limitation amber, cobalt, or vintage green.
Aspect 40. In a particular embodiment of the kit-of-parts according to aspect 39, the packaging unit comprises 28 containers or a multitude of 28 containers, such as 2 to 12 times 28 containers.

The above and further aspects and preferred embodiments of the invention are described in the following sections and in the appended claims. The subject matter of the appended claims is hereby specifically incorporated in this specification.

### BRIEF DESCRIPTION OF THE FIGURES

FIG. 1. Arithmetic Mean (+Standard Deviation (SD)) Plasma Concentrations versus Time Profiles for E4 (Linear Scale) up to 24 h post dose (Pharmacokinetic Set). SD=standard deviation; LLOQ=lower limit of quantitation. Note: LLOQ=0.025 ng/mL. X-axis: Time post dose (hrs); Y-axis: Plasma concentration (ng/ml). Circles: Normal hepatic function group; filled circles: Mild hepatic impairment group; triangles: Moderate hepatic impairment group; filled triangles: Severe hepatic impairment group.

FIG. 2. Geometric Mean Plasma Concentration versus Time Profiles for E4 (Semilogarithmic Scale) (Pharmacokinetic Set). LLOQ=lower limit of quantitation. Note: LLOQ=0.025 ng/ml. X-axis: Time post dose (hrs); Y-axis: Plasma concentration (ng/ml). Circles: Normal hepatic function group; filled circles: Mild hepatic impairment group; triangles: Moderate hepatic impairment group; filled triangles: Severe hepatic impairment group.

FIG. 3. Arithmetic Mean (+SD) Plasma Concentrations versus Time Profiles for E4-3-glucuronide. (Linear Scale) (Pharmacokinetic Set) up to 24 h post dose. SD=standard deviation; LLOQ=lower limit of quantitation. Note: LLOQ=0.250 ng/ml. X-axis: Time post dose (hrs); Y-axis: Plasma concentration (ng/ml). Circles: Normal hepatic function group; filled circles: Mild hepatic impairment group; triangles: Moderate hepatic impairment group; filled triangles: Severe hepatic impairment group.

FIG. 4. Geometric Mean Plasma Concentrations versus Time Profiles for E4-3-glucuronide (Semi-logarithmic Scale) (Pharmacokinetic Set). LLOQ=lower limit of quantitation. Note: LLOQ=0.250 ng/ml. X-axis: Time post dose (hrs); Y-axis: Plasma concentration (ng/ml). Circles: Normal hepatic function group; filled circles: Mild hepatic impairment group; triangles: Moderate hepatic impairment group; filled triangles: Severe hepatic impairment group.

FIG. 5. Arithmetic Mean (+SD) Plasma Concentrations versus Time Profiles of E4-16-glucuronide (Linear Scale) (Pharmacokinetic Set) up to 24 h post dose. SD=standard deviation; LLOQ=lower limit of quantitation. Note: LLOQ=0.500 ng/ml. X-axis: Time post dose (hrs); Y-axis: Plasma concentration (ng/ml). Circles: Normal hepatic function group; filled circles: Mild hepatic impairment group; triangles: Moderate hepatic impairment group; filled triangles: Severe hepatic impairment group.

FIG. 6. Geometric Mean Plasma Concentrations versus Time for E4-16-glucuronide (Semi-logarithmic Scale) (Pharmacokinetic Set). LLOQ=lower limit of quantitation. Note: LLOQ=0.500 ng/ml. X-axis: Time post dose (hrs); Y-axis: Plasma concentration (ng/ml). Circles: Normal hepatic function group; filled circles: Mild hepatic impairment group; triangles: Moderate hepatic impairment group; filled triangles: Severe hepatic impairment group.

FIG. 7. Primary Pharmacokinetic Parameters for E4 and Child-Pugh Score (Pharmacokinetic Set). AUCinf=area under the concentration-time curve from time zero extrapolated to infinity; Cmax=maximum observed plasma concentration. White circles: Normal hepatic function group; filled circles: Mild hepatic impairment group; Squares: Moderate hepatic impairment group; Triangles: Severe hepatic impairment group; X-axis: Child-Pugh Score; Y-axis: Cmax (ng/mL); AUCinf (h*ng/ml).

FIG. 8. Geometric LS Mean Ratios of Cmax for E4. X-axis: Geometric LS Mean Ratio (Cmax (ng/ml) and 90% CI; Y-axis: Comparison (Test vs. Reference). Top comparison: Mild vs Normal. Middle comparison: Moderate vs Normal. Bottom comparison: Severe vs Normal.

FIG. 9. Geometric LS Mean Ratios of AUCinf for E4. X-axis: Geometric LS Mean Ratio (AUCinf (h*ng/mL)) and 90% CI; Y-axis: Comparison (Test vs. Reference). Top comparison: Mild vs Normal. Middle comparison: Moderate vs Normal. Bottom comparison: Severe vs Normal.

FIG. 10. Vasomotor symptoms (VMS) (measured by number of hot flushes per week) frequency in 15 mg estetrol monohydrate, 20 mg estetrol monohydrate, and placebo treatment group. Statistical analysis via MMRM model of Change from Baseline to Week 4 and Week 12 for weekly Frequency of moderate to severe VMS; Intent-to-treat (ITT)-population; p-values between E4 treatments and placebo on the change from Baseline versus placebo (difference of LS means).

FIG. 11. Proportion of subjects with at least 50% or 75% reduction from baseline in the weekly frequency of moderate to severe VMS over time; Efficacy Study Part-Intent-to-treat (ITT) population; *: p-value<0.05; **: p-value<0.01; ***: p-value<0.001; ****: p-value<0.0001.

FIG. 12. VMS severity in 15 mg estetrol monohydrate, 20 mg estetrol monohydrate, and placebo treatment group. Statistical analysis via MMRM model of Change from Baseline to Week 4 and Week 12 for weekly severity of moderate to severe VMS by FDA method; Intent-to-treat (ITT) population; p-values between E4 treatments and placebo on the change from baseline versus placebo (difference of LS means).

FIG. 13. Haemoglobin A1C levels are decreased in subjects upon 15 mg and 20 mg estetrol monohydrate treatments after 12 weeks. (A) Haemoglobin A1C levels. Left condition=baseline, right condition=week 12 (B) Change from baseline. *: p-value<0.05; **: p-value<0.01; ***: p-value<0.001; ****: p-value<0.0001.

FIG. 14. Fasting glucose levels are decreased in subjects upon 15 mg and 20 mg estetrol monohydrate treatments after 12 weeks. (A) Fasting glucose levels. Left condition=baseline, right condition=week 12 (B) Change from baseline. *: p-value<0.05; **: p-value<0.01; ***: p-value<0.001; ****: p-value<0.0001.

### DETAILED DESCRIPTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints. This applies to numerical ranges irrespective of whether they are introduced by the expression “from . . . to . . . ” or the expression “between . . . and . . . ” or another expression.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g. any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation or meaning is meant to apply throughout this specification, i.e. also in the context of other aspects or embodiments of the invention, unless otherwise defined. For example, embodiments directed to products are also applicable to corresponding features of methods and uses.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, alternative combinations of claimed embodiments are encompassed, as would be understood by those in the art.

Unless indicated otherwise, all methods, steps, techniques and manipulations that are not specifically described in detail can be performed and have been performed in a manner known per se, as will be clear to the skilled person. Reference is for example again made to standard handbooks as well as to the general background art referred to herein and to the further references cited therein.

The term “estetrol component”, as used throughout this document, encompasses substances selected from the group consisting of estetrol, esters of estetrol, esters of estetrol wherein the hydrogen atom of at least one of the hydroxyl groups has been substituted by an acyl radical of a hydrocarbon carboxylic, sulfonic acid or sulfamic acid of 1-25 carbon atoms, estetrol hydrates such as estetrol monohydrate; and combinations thereof. It is understood that when estetrol is mentioned throughout any section of this specification, any estetrol-containing component (i.e. compound) and/or estetrol derivative (such as an estetrol ester) is also envisaged. More preferably, in the context of the present disclosure, a particularly preferred estetrol component suitable for the dosage units, medical uses and methods of treatment described herein is estetrol (including estetrol hydrates). Most preferably, said estetrol component is estetrol monohydrate.

The term “estetrol” as used herein refers to 1,3,5 (10)-estratrien-3,15alpha, 16alpha, 17beta-tetrol or 15alpha-hydroxyestriol as well as hydrates of estetrol, e.g. estetrol monohydrate. “Estetrol”, or short “E4” is an estrogen steroid produced by the foetal human liver (PubChem CID: 27125). Estetrol may be described as a 3-hydroxy steroid corresponding to 17beta-estradiol wherein the 15a and 16a positions are substituted for two additional hydroxy groups. It is known that estetrol is an estrogen receptor agonist (Coelingh Bennink et al., Climacteric, 2008). The estetrol may be chemically synthesised, synthesised by the use of (mutant) recombinant enzymes, or synthesised by any combination thereof. It is therefore evident that the terms “estetrol” and “estetrol components” equally encompasses further chemically modified estetrol. Estetrol may be indicated in the art by its molecular formula: C₁₈H₂₄O₄, or by structural formula (I).

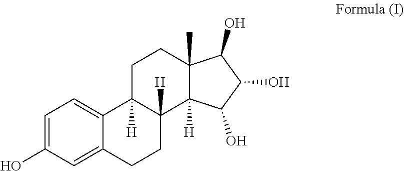

In preferred embodiments the estetrol component is estetrol or an ester thereof. In further embodiments, the estetrol component is estetrol monohydrate. A skilled person appreciates that estetrol monohydrate corresponds to estetrol containing one molecule of water, and that the core structural formula of estetrol does not differ from Formula (I). By means of illustration and not limitation, the structural formula of estetrol monohydrate is indicated by Formula (II):

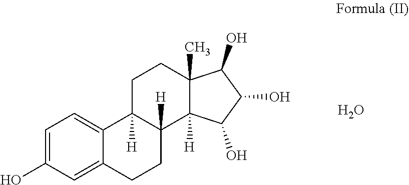

The term “subject”, or “patient” as used herein refers to female human subjects, preferably peri- and/or post-menopausal female subjects. The female subject envisaged herein may be subjects in need of, or deemed in need of a treatment for alleviating estrogen deficiency symptoms (such as menopause-associated symptoms), or predicted to be in need of such a treatment in a foreseeable future point in time due to for example entering the peri-menopausal stage of life.

A skilled person is aware that terms such as “quantity”, “amount” and “level” are synonyms and have a well-defined meaning in the art. The terms as used herein may particularly refer to an absolute quantification of a molecule such as a steroid, in (a sample taken from) a subject, or to a relative quantification of a molecule or analyte in a sample, i.e., relative to another value such as relative to a reference value as taught herein, or to a range of values indicating a base-line of a certain parameter. These values or ranges of values may be obtained from one single subject or from a group of subjects (i.e. at least two subjects). Further, when reference is made to (absolute or relative) quantities of parameters such as AUC or Cₘₐₓ, these amounts are to be interpreted as the quantity as measured in (a sample of) the blood plasma of one or more subjects. Both “blood plasma” and “serum plasma” are generally accepted terms in medicinal and clinical contexts and leave no ambiguity with respect to their interpretation to a skilled person (Matthew and Varacallo, Physiology, Blood plasma, StatPearls, 2019).

“Dosage unit”, interchangeably used with “dosage form” herein and in the art indicates a physical preparate that is suitable for administration to a subject, without the necessity to adapt the drug product prior to administration, i.e. the final beneficial product. A dosage unit therefore indicates a ready-to-administer composition. The term is not limiting for any other particulars of the treatment, such as frequency of administration and/or any characteristic of the dosage unit (taste, appearance, size, etc.). In the context of the present invention, each dosage unit preferably comprises an estetrol component in an amount equivalent to from about 15 mg to about 25 mg of estetrol as pharmaceutically acceptable ingredient. The presence of an estetrol component as pharmaceutically acceptable ingredient does not exclude the presence of one or more further pharmaceutical ingredients and/or pharmaceutically acceptable ingredients in the dosage unit. The term “pharmaceutically acceptable” as used herein is consistent with the art and means compatible with the other ingredients of a pharmaceutical composition and not deleterious to the recipient thereof. Non-limiting suitable excipients are described further throughout the disclosure.

A skilled person appreciates the meaning of the following abbreviations that are used throughout the present disclosure which should each be interpreted according to their commonly accepted meaning:
Cₘₐₓ maximum observed plasma concentration;
AUC_{inf} area under the concentration-time curve (AUC) from time zero extrapolated to infinity;
AUC_{0-24 h} AUC from time 0 to 24 h post-dose;
AUCₗₐₛₜ AUC up to the last time with concentration above the lower limit of quantitation;
tₘₐₓ time to attain Cmax;
t_{1/2} terminal elimination half-life;
C_{L/F} apparent clearance (only for E4);
λ_{z} terminal elimination phase rate constant;
Vz/F apparent volume of distribution during terminal phase (only for E4).

Through extensive research, the inventors have observed that estetrol surprisingly provides an excellent safety profile when used to treat estrogen deficiency symptoms such as but not limited to menopause-associated symptoms in subjects that have a hepatic impairment. This is in contrast to existing commercial products such as Angeliq® (containing 1 mg estradiol and 0.5 mg drospirenone) that even indicate hepatic impairment as a clear contraindication for using the product. Notably, this improved safety was obtained while maintaining a satisfying efficacy. The degree of hepatic impairment that the subjects have is not particularly limiting for the present invention, and favorable pharmacokinetic properties could be identified to some extent throughout any group as stratified by Child-Pugh grouping, although the unexpected properties are most pronounced in subject characterized by a mild or moderate hepatic impairment. Particular favourable parameters include the maximum observed plasma concentration (Cₘₐₓ) of the estetrol component, area under the concentration-time curve (AUC) from time zero extrapolated to infinity (AUC_{inf}) of the estetrol component, AUCinf of E4-3-glucuronide, and half-life times of the estetrol component and its metabolites that are obtained after administration of estetrol to hepatically impaired subjects.

Hence, in a first aspect the invention is directed to a composition for use in alleviating estrogen deficiency symptoms (which may optionally present themselves in a context of menopause) in a hepatically impaired subject, wherein said composition comprises an estetrol component and wherein said composition is administered at a daily amount equivalent to from about 15 mg to about 25 mg of estetrol.

A skilled person appreciates that the expression “administered at a daily amount equivalent to from about x mg to about y mg of estetrol” indicates the administration of a substance, in the context of the present invention an estetrol component, that achieves the same physiological and/or psychological effects as a situation wherein the subject would have been administered x mg to y mg of estetrol. Additionally, the term “daily” indicates that the recited amounts are the cumulative amount that is administered to a subject per day. A skilled person understands that if the estetrol component is administered only once per day (i.e. daily), that the amount of estrogen administered in that single administration will be the daily dose. Alternatively, a skilled person appreciates that if the estetrol component is administered more than once per day (e.g. 2 times or 3 times) the daily amount will correspond to the sum of estetrol component administered during each administration event within a total time window of 24 hours. In embodiments where different estetrol components are comprised in the composition (e.g. estetrol monohydrate and an estetrol ester), it is within the capacities of a skilled person to verify the amount of estetrol each estetrol component corresponds to. Preferred embodiments within the context of the invention comprise the administration of a single estetrol component, such as but not limited to estetrol monohydrate.

Additionally, the invention envisages use of a composition that comprises an estetrol component for the manufacturing of a medicament for alleviating estrogen deficiency symptoms such as menopause-associated symptoms in a hepatically impaired subject, wherein the medicament is formulated to be administered such that a daily amount of estetrol component is administered that is equivalent to from about 15 mg to about 25 mg of estetrol.

Additionally, the use of a composition comprising an estetrol component for alleviating estrogen deficiency symptoms such as menopause-associated symptoms in a hepatically impaired subject is envisaged, wherein the composition is used such that the estetrol component is administered to a subject in a daily amount equivalent to from about 15 mg to about 25 mg of estetrol.

Additionally, a method of alleviating (i.e. treating) estrogen deficiency symptoms such as menopause-associated symptoms in a hepatically impaired subject is envisaged. The method comprises the step of administering a composition comprising an estetrol component at a daily amount equivalent to from about 15 mg to about 25 mg of estetrol to said subject.

“Estrogen deficiency symptoms” have been described in the art and encompass any symptom a subject can experience due to estrogen deficiency (i.e. “hypoestrogenism”, or “estrogen deficiency syndrome”). In a preferred embodiments of the invention, the estrogen deficiency symptoms are occurring in a context of menopause and hence the symptoms may be referred to as menopause-associated symptoms. The expression “alleviating menopause-associated symptoms” as used throughout the present specification has a well-established meaning within the technical field and indicates treatment of any unwanted physical or psychological manifestation that accompanies menopause, or the onset of menopause. Thus, the composition disclosed herein is typically used as a (therapeutic) treatment for menopause-associated symptom, i.e. as a composition that is administered to a subject in a context of therapy. Also envisaged is the use of the composition described herein for preventing the onset of estrogen deficiency symptoms or more specifically menopause-associated symptoms.

Moreover, a skilled person appreciates that the expression “alleviating menopause-associated symptoms” combined with the feature of an estetrol component-comprising composition implies that the present uses and methods described herein additionally encompass the medical indication of hormone replacement therapy. It is commonly accepted in the technical field of pharmacy and medicine that hormone replacement therapy (abbreviated as “HRT”) involves the use of one or more medications (i.e. one or more compositions) designed to increase or supplement hormone levels in women who lack adequate hormone production. HRT can mitigate and prevent symptoms caused by diminished circulating estrogen (and progesterone hormones), regardless as to whether the subject is premenopausal, peri-menopausal, menopausal or post-menopausal. Hormone replacement therapy (HRT) is used to describe either unopposed estrogen use (for female subjects who have undergone hysterectomy) or combined estrogen-progestin therapy (for female subjects still having a uterus). Further specific disease states have been documented for each stage of menopausal progression.

The terms “treatment” or “treat” are to be interpreted as both the therapeutic treatment of a symptom, disease or condition that has already developed, leading to (clinical) manifestations, as well as prophylactic or preventive measures, wherein the goal of the treatment is to prevent, lessen, or reduce the chances of incidence of an undesired affliction, such as to prevent occurrence, development and progression of symptoms, (clinical) conditions related to menopause. Beneficial or desired clinical results may include, without limitation, alleviation of one or more symptoms, improvement of one or more biological markers, diminishment of the extent (i.e. a reduction in severity) of the menopause-associated symptoms, stabilized (i.e. not worsening) of menopausal symptoms, delay or slowing of the manifestation of menopause-associated symptoms, and the like. “Prevention” or “prevent” as used in the context of the invention refers to an aversion of manifestation of a condition or disease image in a subject, i.e. the establishment of preventive measures or prophylactic measures. Preventive treatment refers to treatments wherein the object is to avoid a subject's body or an element thereof to show (worsening of) symptoms of an undesired physiological or psychological change induced by menopause. As used herein, the terms “therapeutic treatment” or “therapy” and the like, refer to treatments wherein the aim is to change a subjects body or a part of a subjects body from an undesired physiological state, disease or disorder which is caused by aging, to a desired state, such as a less severe state (e.g., amelioration, or even back to its normal, healthy state (e.g., restoring the health, the physical integrity and the physical well-being of a subject), to keep it (i.e., not worsening) at said undesired physiological status (e.g., stabilization), or slow down progression to a more severe or worse state compared to said undesired physiological change or disorder). Measurable lessening includes any statistically significant decline in a measurable marker or symptom. Statistically significant as used herein refers to p values below 0.05, which is a commonly accepted cut-off score in statistical analysis as a skilled person appreciates. “Treatment” encompasses both curative treatments and treatments directed to reduce estrogen deficiency symptoms and/or slow progression and/or stabilize the estrogen deficiency symptoms.

The subjects preferably envisaged by the present invention are female subjects that are characterised by a hepatic impairment (either diagnosed, considered, or predicted (i.e. prognosticated)) and are experiencing or are predicted (i.e. prognosticated) to experience one or more estrogen deficiency symptoms which can optionally be considered as one or more menopause-associated symptoms.

In embodiments wherein the subject is already experiencing one or more menopause-associated symptoms, the subject may a hormone deregulated subject. In the context of the present invention, the deregulation is determined or established by comparison of the hormone level of the subject with a representative value of an adult, healthy female subject. The cause of the hormone deregulation is particularly limiting for the present disclosure, and may be of a natural cause (such as menopausal hormonal changes) but may equally be caused by a pathologic condition or deficiency. In certain embodiments, the hormone deregulation is an estrogen deregulation, such as an 17β-estradiol deregulation. In such embodiments, the 17β-estradiol differs at least 15%, preferably at least 25%, more preferably at least 50% from a representative 17β-estradiol value for female adult subjects. In cases wherein the endogenous estrogen production of the female subject is reduced, the subject may be considered to be a subject suffering from estrogen-deficiency syndrome.

The term “17β-estradiol” refers to (17beta)-estra-1,3,5 (10)-triene-3,17-diol and may be interchangeably be indicated by the terms “oestradiol”, or “E2”, and is an estrogen endogenously produced by the human body. More particularly, estradiol is the predominant estrogen hormone produced by the human ovaries during the initial half of the menstrual cycle (i.e. the follicular phase). Estradiol is also involved in the maintenance of bone density, reduction of vasomotor symptoms, and maintenance of the normal structure of the female genital organs in menopausal subjects.

Embodiments concerning subjects characterised by an estrogen depletion (i.e. hypoestrogenism, or estrogen deficiency syndrome) are envisaged by the invention. While the invention is primarily directed to alleviating menopause-associated symptoms, a skilled person will appreciate that numerous of these symptoms can also present themselves in a female subject that has a medical condition which is not menopause but nonetheless results in one or more menopause-associated symptoms. Hence, the invention is equally directed to alleviating estrogen deficiency symptoms that may present themselves in a person that is not undergoing menopause, and/or is not predicted by an imminent onset of menopause. The cause of the hypoestrogenism is not particularly limited and may therefore be caused by the non-limiting causes of menopause, hypogonadism, castration, primary ovarian failure, and aromatase inhibitor or gonadotropin-releasing hormone analogue breast cancer treatment.

In certain embodiments, the subject is a female menopausal, perimenopausal, or postmenopausal subject. In certain embodiments, the subject is a menopausal, perimenopausal, or postmenopausal subject having an 17β-estradiol level of less than 100 μg/ml, preferably less than 50 μg/ml, preferably less than 30 μg/ml, more preferably less than 20 μg/ml, more preferably less than 20 μg/ml, most preferably less than 10 μg/ml. In alternative embodiments, the subject is a menopausal, perimenopausal, or postmenopausal subject that is characterised by having follicle-stimulating hormone concentrations of at least 20 milli-international units per millilitre (mlU/ml), preferably at least 25 mlU/ml, more preferably at least 30 mlU/ml, more preferably at least 35 mlU/ml, most preferably at least 40 mlU/ml.

“Menopausal subjects”, used interchangeably in the art with “post-menopausal subjects” or “climacteric subjects” are female subjects that that have not had menstrual bleeding for a year which is accompanied by a decrease or discontinuation in hormone production by the ovaries (such as 17β-estradiol).

According to the US FDA, the criteria for post-menopausal status are:
At least 12 months of spontaneous amenorrhea; or
At least 6 months of spontaneous amenorrhea with serum FSH levels>40 mlU: mL; or.
At least 6 weeks postsurgical bilateral oophorectomy with or without hysterectomy.

Alternatively worded, “menopause” may be described as a biological condition characterised by impairment or cessation of ovarian primary function. Menopause may be accompanied by a broad range of clinical symptoms which are variable in severity such as but not limited to vasomotor dysfunction, vaginal dryness, mood changes, sleep disturbances, urinary incontinence, cognitive changes, somatic complaints, and sexual dysfunction. Methodologies to diagnose menopause have been described in the art and are therefore known to a person skilled in the art (Nelson, Menopause, Lancet, 2008).

“Perimenopause” refers to a period of life which begins approximately three to four years prior to menopause and ends one year after the final menstrual period, and is characterised by persistent irregular menstrual cycles, extreme fluctuations in hormonal levels, frequent anovulation and the appearance of vasomotor symptoms (Harlow et al., Executive summary of the Stages of Reproductive Aging Workshop+10: addressing the unfinished agenda of staging reproductive aging, Menopause, 2012). The term “post menopause” or “postmenopausal” is indicative for female subjects that are characterised by a permanent cessation of menstrual periods. This permanent cessation is determined retrospectively after an observation of 12 months of amenorrhea without any other obvious pathological or physiological cause. The term “post menopause” also includes menopause as the consequence of premature ovarian failure, surgery (ovariectomy for example), chemotherapy or radiotherapy for cancer, and certain diseases (for example, infections or hypothyroidism).

Thus, in the context of the present invention the female subject is a menopausal, perimenopausal, or post-menopausal subject.

Preferably, the subject is a female subject of adult age. More preferably, the subject is a female subject of middle age or elder age. Yet more preferably, the subject is a female subject of at least 40 years of age, preferably of at least 50 years of age, preferably of at least 55 years of age, more preferably of at least 60 years of age, more preferably of at least 65 years of age. Alternatively, the subject may be a female subject of between 40 and 90 years, preferably of between 45 and 85 years of age, preferably of between 50 and 80 years of age, more preferably of between 55 and 75 years of age, more preferably of between 60 and 70 years of age or between 65 and 75 years of age. In certain embodiments, the female subject is at most 90 years of age, preferably at most 85 years of age, more preferably at most 80 years of age, more preferably at most 75 years of age, more preferably at most 70 years of age, more preferably at most 65 years of age, more preferably at most 60 years of age. “Diagnosed with”, “diagnosing”, and diagnosis are indicative for a process of recognising, deciding on, or concluding on a disease, condition, or (adverse side effect) in a subject on the basis of symptoms and signs and/or from results of various diagnostic procedures (such as, for example, from knowing the presence, absence and/or quantity of one or more biomarkers of or clinical symptoms characteristic for the diagnosed disease or condition). “Diagnosis of” one or more estrogen deficiency symptoms may particularly mean that the subject has at least one estrogen deficiency symptom as adjudged by a skilled medical practitioner. Although a subject presents one or more conventional symptoms or signs indicative thereof, an absence of symptoms may ultimately be diagnosed. “Diagnosis of” one or more of the estrogen deficiency symptoms herein in a subject may particularly mean that the subject has at least one physiological estrogen deficiency symptom and/or at least one psychological estrogen deficiency symptom. “Prognosticating” in the context of the invention is indicative for anticipation on the progression of one or more estrogen deficiency symptoms in a subject and the prospect (e.g. the probability, duration, and/or extent) of recovery, and/or the severity of experiencing or amelioration of said one or more estrogen deficiency symptoms. The term may encompass anticipation of not further worsening or aggravating of such, preferably within a given time period. The term “a poor prognosis of” the disease or condition typically encompasses an anticipation of a substandard recovery and/or unsatisfactorily slow recovery, or no recovery at all, or further worsening of the one or more estrogen deficiency symptoms, which encompasses both physiological estrogen deficiency symptoms, psychological estrogen deficiency symptoms, or a combination thereof.

Related to the foregoing, “predicting” or “prediction” generally refer to a statement, declaration, indication or forecasting of a disease or condition in a subject not (yet) showing any, or a limited, clinical manifestation of one or more estrogen deficiency symptoms. A prediction of one or more estrogen deficiency symptoms in a subject may indicate a probability, chance, or risk that said subject will develop said clinical manifestation, condition, or (adverse) side effect, for example within a certain time period after diagnosis of the one or more estrogen deficiency symptoms. Said probability, chance or risk may be indicated as any suitable qualitative or quantitative expression, wherein non-limiting examples of a quantitative expression include absolute values, ranges or statistics. Alternatively, probabilities, chances, or risks may be indicated relative to a suitable control subject or group of control subject (i.e. a control subject population (such as, e.g., relative to a general, normal or healthy subject or subject population)). Therefore, any probability, chance or risk may be advantageously indicated as increased or decreased, upregulated or downregulated, as fold-increased or fold-decreased relative to a suitable control subject or subject population, or relative to a baseline value which may be derived from either a control subject (population), textbook reference values. It is evident that when a population of subjects is used to define the baseline value, said baseline value will be a centre size of one or more values (parameters) of a population, such as the mean or median of said value. A skilled person further appreciates that monitoring may be applied in the course of a medical treatment of a subject. Such monitoring may be comprised, e.g., in decision making whether a patient may be discharged from a controlled clinical or health practice environment, needs a change in treatment or therapy, or requires hospitalisation.

In each of the embodiments disclosed herein, the composition aims to alleviate estrogen deficiency symptoms such as menopause-associated symptoms by providing an estetrol component in an amount which is equivalent to from about 15 mg to about 25 mg of estetrol. This implies that the estetrol component in the composition is present in a pharmaceutically effective amount, including in such embodiments wherein the daily equivalent dose is obtained by multiple administrations of the composition. “A pharmaceutically effective amount” refers to an amount necessary to obtain a physiological effect and may indicate a therapeutically effective amount and/or a prophylactically effective amount. The physiological effect may be achieved by a single dose or by multiple doses. A “therapeutically effective amount” or “therapeutically effective dose” indicates an amount of estetrol component that when administered brings about a clinical positive response with respect to treatment of a subject afflicted by estrogen deficiency symptoms. Similarly, a “prophylactically effective amount” or “prophylactically effective dose” refers to an amount of estetrol component that inhibits or delays the onset of clinical manifestation of condition as being sought by a researcher, veterinarian, medical doctor or other clinician. A skilled person is aware that terms such as “quantity”, “amount” and “level” are synonyms and have a well-defined meaning in the art and appreciates that these may particularly refer to an absolute quantification of an estetrol component which is considered an effective amount for the applications described herein, or to a relative quantification of the estetrol component. Suitable values or ranges of values may be obtained from one single subject or from a group of subjects (i.e. at least two subjects).

A further aspect of the invention is directed to a method to improve the safety of therapy for estrogen deficiency symptoms (such as but not limited to menopause-associated symptoms) in subjects with hepatic impairment, wherein said method comprises administration of a composition comprising an estetrol component at a daily amount equivalent to from about 15 mg to about 25 mg. As exemplified by the Examples enclosed herewith, it is evident that administration of an estetrol component improves the safety of therapy for estrogen deficiency symptoms by reducing the hepatic burden on the subject when compared to existing estrogen replacement therapies that rely on administration of an estrogen different from the estetrol component envisaged herein. Thus, the invention is also directed to a method of improving the pharmacokinetic profile of a drug for treating estrogen deficiency symptoms, and a method of improving safety of a treatment directed to alleviating estrogen deficiency symptoms due to the improved pharmacokinetic profile in an hepatically impaired subject.

The subjects described herein are hepatically impaired subjects. Unless explicitly stated otherwise, the degree of hepatic impairment is not particularly limiting in the context of the invention. Throughout the present description, hepatic impairment may be expressed by means of the Child-Pugh Score. Alternative names for this scoring system have been used in the art and include but are not limited to “Child-Turcotte-Pugh score” or “Child criteria”. The Child-Pugh score enjoys widespread use throughout the art (Tsoris and Marlar, Updated 2022 Mar. 18, StatPearls) and the classification schedule for hepatic impairment is summarized below, as known to a person skilled in the art.

### Classification of Hepatic Function—Child-Pugh Score

| | 1 point | 2 points | 3 points |
|---|---|---|---|
| Albumin (g/L) | >35 | 35-28 | <28 |
| | (>3.5 g/dL) | (3.5-2.8 g/dL) | (<2.8 g/dL) |
| Total bilirubin (μmol/L) | <34.2 | 34.2-51.3 | >51.3 |
| | (<2 mg/dL) | (2-3 mg/dL) | (>3 mg/dL) |
| Prothrombin time | <4 | 4-6 | >6 |
| prolongation (s) | | | |
| Ascites | Absent | Slight | Moderate to severe |
| Encephalopathy*^{,†} | Absent | Grade 1 or 2 | Grade 3 or 4 |

Interpretation:
Grade A: 5-6 points;
Grade B: 7-9 points;
Grade C: 10-15 points
*Assessment of encephalopathy is primarily based on clinical signs and symptoms as detailed below; an encephalogram is not obligatory for grading of encephalopathy
^{†}Grade 0: normal consciousness, personality, neurological examination, electroencephalogram
Grade 1: restless, sleep disturbed, irritable/agitated, tremor, impaired handwriting, 5 cps waves
Grade 2: lethargic, time-disoriented, inappropriate, asterixis, ataxia, slow triphasic waves
Grade 3: somnolent, stuporous, place-disoriented, hyperactive reflexes, rigidity, slower waves
Grade 4: unrousable coma, no personality/behavior, decerebrate, slow 2-3 cps delta activity

Hepatic function groups are defined as follows:
Group 1: normal hepatic function.
Group 2: mild hepatic impairment (Child-Pugh score 5-6 points).
Group 3: moderate hepatic impairment (Child-Pugh score 7-9 points)
Group 4: severe hepatic impairment (Child-Pugh score 10-14 points)

Preferred subjects in the context of the invention are subjects that have a grade of encephalopathy that is grade 2 or less, preferably subjects that have a grade of encephalopathy that is grade 1 or less, more preferably subjects that have a grade of encephalopathy that is grade 0.

Optionally, the subject is a female menopausal, perimenopausal, or postmenopausal subject characterized by a hepatic impairment corresponding to a Child-Pugh score of at least 5 points. In preferred embodiments, the subject is a female menopausal, perimenopausal, or postmenopausal subject characterized by a hepatic impairment corresponding to a Child-Pugh score of at least 6 points, preferably at least 7 points, preferably at least 8 points, preferably at least 9 points, preferably at least 10 points, preferably at least 11 points, preferably at least 12 points, preferably at least 13 points, preferably at least 14 points. Hence, the subject may be a female menopausal, perimenopausal, or postmenopausal subject characterized by a hepatic impairment corresponding to a Child-Pugh grade A, grade B, or grade C. In certain embodiments, the subject is a female menopausal, perimenopausal, or postmenopausal subject characterized by a hepatic impairment corresponding to a Child-Pugh score of from 5 points to 15 points, preferably of from 5 points to 14 points, preferably of from 5 points to 13 points, preferably of from 5 points to 12 points, preferably of from 5 points to 11 points, preferably of from 5 points to 9 points, preferably of from 5 points to 8 points, preferably of from 5 points to 7 points, preferably of from 5 points to 6 points.

In alternative embodiments, the subject is a female menopausal, perimenopausal, or postmenopausal subject characterized by a hepatic impairment corresponding to a Child-Pugh score of from 5 points to 15 points, preferably of from 6 points to 15 points, preferably of from 7 points to 15 points, preferably of from 8 points to 15 points, preferably of from 9 points to 15 points, preferably of from 10 points to 15 points, preferably of from 11 points to 15 points, preferably of from 12 points to 15 points, preferably of from 13 points to 15 points, preferably of from 14 points to 15 points.

In certain embodiments, the subject is a female menopausal, perimenopausal, or postmenopausal subject characterized by a mild hepatic impairment (i.e. a Child-Pugh score corresponding to “grade A”). In alternative embodiments, the subject is a female menopausal, perimenopausal, or postmenopausal subject characterized by a moderate hepatic impairment (i.e. a Child-Pugh score corresponding to “grade B”). In yet alternative embodiments, the subject is a female menopausal, perimenopausal, or postmenopausal subject characterized by a severe hepatic impairment (i.e. a Child-Pugh score corresponding to “grade C”). In preferred embodiments, the subject is a female menopausal, perimenopausal, or postmenopausal subject characterized by a mild or moderate hepatic impairment (i.e. a Child-Pugh score corresponding to “grade A” or “grade B”).

It is evident that the expressions “estrogen deficiency symptoms” and “menopause-associated symptoms” both comprise psychological symptoms, physiological symptoms, and any combination thereof.

Psychological estrogen deficiency symptoms and menopause-associated symptoms include by means of illustration and not limitation depression, irritability, mood changes, insomnia, sleep disturbance, anxiety, nervous tension and any combination thereof. It is evident that the term “psychological menopause-associated symptoms” additionally encompasses any form or degree of emotional distress due to one or more physical menopause-associated symptoms occurring in said subject.

Physiological estrogen deficiency symptoms and menopause-associated symptoms include by means of illustration and not limitation joint pain, loss of bone density, urinary tract infections, urinary incontinence, dryness of the vagina, uterine prolapse, changes in skin texture, weight gain, dyspareunia, cardiovascular diseases, diabetes and any combination thereof. Optionally, the diabetes is diabetes type 2 (e.g. described in Mauvais-Jarvis et al., Endocr Rev, 2017).

Preferably, the compositions, uses, and methods described herein are for use in reducing VMS frequency, VMS severity, hot flush weekly weighted score, dryness of the vagina, dyspareunia, or any combination thereof, or for use in improving Quality of Life according to the Menopause Rating Scale (MRS) and/or the Menopause-specific Quality of Life (MENQOL) questionnaires.

The term “VMS” as used herein (alternatively in its non-abbreviated form, “vasomotor symptoms”) corresponds to thermo-regulatory disturbances characteristic of menopause. The term VMS encompasses hot flashes (interchangeably indicated by the term “hot flushes”), sweating attacks such as night sweats, chills and increased perspiration, and palpitations. VMS are episodes of profuse heat accompanied by sweating and flushing, experienced predominantly around the head, neck, chest, and upper back. VMS are classified into mild, moderate and severe categories. In certain embodiments the VMS are selected from the group consisting of: hot flushes, night sweats, sleep disturbances, mood swings, and any combination thereof.

A severity scoring system to define the different severity categories of VMS is commonly used in the technical field:
a score of mild (1) for a sensation of heat without sweating;
a score of moderate (2) for a sensation of heat with sweating/the subject is able to continue activity; and
a score of severe (3) for a sensation of heat with sweating/which causes cessation of activity.

In addition, a severity score of zero is attributed to the patients who have experienced a 100% VMS relief during a given week. From these score records, the VMS severity at baseline is typically calculated by taking into account only moderate and severe VMS, such that the total number of moderate VMS during the 7 days of the baseline week is multiplied by 2 and added to the total number of severe VMS during the 7 days of the baseline week multiplied by 3. This total is then divided by the total number of moderate and severe VMS during the baseline week.

The severity at weeks 4 and 12 is calculated using the following formula for each of these weeks:
the number of mild VMS during 7 days multiplied by 1;
the number of moderate VMS during 7 days multiplied by 2;
the number of severe VMS during 7 days multiplied by 3;
   and adding the 3 resulting numbers together before dividing this total by the total number of mild, moderate and severe VMS during the 7 days of the week.

VMS Severity is assessed as follows: the severity at baseline is also calculated by taking into account only moderate and severe VMS, such that the total number of moderate VMS during the 7 days of the baseline week is multiplied by 2 and added to the total number of severe VMS during the 7 days of the baseline week multiplied by 3. This total is then divided by the total number of moderate and severe VMS during the baseline week.

The severity at weeks 4 and 12, however, is calculated using the following formula for each of these weeks:
the number of moderate VMS during 7 days multiplied by 2;
the number of severe VMS during 7 days multiplied by 3.
   and adding the 2 resulting numbers together before dividing this total by the total number of moderate and severe VMS during the 7 days of the week.

In another embodiment, the severity score can be calculated according to the method described in Archer et al. (Menopause, 2014). According to this method, a daily severity score is calculated using the following formula for each day during 7 days:
the number of moderate VMS during 1 day is multiplied by 2;
the number of severe VMS during 1 day is multiplied by 3;
the 2 resulting numbers are added together;
the addition result is in turn divided by the total number of VMS during the same day (moderate and severe).

The same formula is applied during 7 consecutive days, the 7 resulting numbers are summed together and divided by 7 to calculate the “weekly mean daily severity” score of moderate to severe VMS.

Further, the terms “Hot Flush Weekly Weighted Score”, as used herein, corresponds to a score taking into account frequency and severity which is calculated by using the severity score (as calculated above): [(1×No. of mild VMS)+(2×No. of moderate VMS)+(3×No. of severe VMS)] during a 7 day period.

Such a weighted score was for example used by Notelovitz et al. (Obstetrics and Gynaecology, 2000).

In yet alternative embodiments, any combination of the above described methods maybe employed to evaluate the weekly severity and/or frequency of VMS.

As used herein, the terms “Quality of Life” (and the abbreviation “QoL”) refers to a parameter which can be assessed, for example, by using questionnaires, such as, by way of example and not limitation, the “Menopause Rating Scale” questionnaire (Heinemann et al., 2003, “International versions of the Menopause Rating Scale (MRS)” Health Qual Life Outcomes 1:28; Heinemann et al., 2004, “The Menopause Rating Scale (MRS) scale: A methodological review”. Health Qual Life Outcomes 2:45; Heinemann et al., 2004, “The Menopause Rating Scale (MRS) as outcome measure for hormone treatment? A validation study”. Health Qual Life Outcomes 2:67; as further detailed below in Example 1 Section C.) or the MENQOL questionnaire (The Menopause-specific Quality of Life (MENQOL) questionnaire, Hilditch et al.; Maturitas 1996; A menopause-specific quality of life questionnaire: development and psychometric properties; 24 (3); p. 161-175).

In a particular embodiment, the composition described herein is beneficially administered to take advantage of the absence of effect of the smoking status of the subject on the therapy of the invention. It has indeed long been known that smoking significantly reduces serum estrogen concentrations, such as reported for example in the clinical study analysis of Bjarnason et al. (Bjarnason et al.; Climacteric 2012; Acute and long-term estradiol kinetics in smoking postmenopausal women; 15:5; p. 449-454) who found that in the estrogen group, smoking leads to significantly lower levels of both serum estrone and serum estradiol at all post-randomization time points, while no differences between smokers and non-smokers were seen on placebo. Bjarnason et al. conclude that smoking reduces serum estrogens at both trough and after 2 h in postmenopausal women on estrogen treatment, that the effect of smoking on estrogen concentrations is fully expressed in women smoking ten or less cigarettes daily, and that the influence of smoking upon the metabolism of estrogen therapy is constant and without dose-response for standard smoking intensities.

In this particular embodiment, based on the surprising finding that the therapy of the invention is not affected by the smoking status of the subject, the composition is optionally administered to a patient population smoking 5 or more cigarettes daily, to a patient population smoking 10 or more cigarettes daily, or to a patient population smoking 15 or more cigarettes daily.

In yet another particular embodiment of the invention, the composition described herein is beneficially administered to take advantage of the absence of effect of the BMI of the subject on the therapy of the invention. It has indeed been surprisingly found that contrary to hormone replacement therapies described in the art, the efficacy of the therapy of the invention is not affected by the BMI value of the subject. In this particular embodiment, the hormone therapy of the invention is preferably administered to a subject whose BMI is 25 or more, 28 or more, 30 or more, 33 or more, 35 or more, 37 or more, or 40 or more, In this particular embodiment, the hormone therapy of the invention is preferably administered to an overweight subject, or to an obese subject. As used herein, “BMI” (or “Body Mass Index” in its non-abbreviated form) refers to an index relating to weight and height of a subject, which is calculated by dividing the subject's weight in kilograms by the subject's height in meters squared. A BMI of 27.3 or more classifies the female subject as “overweight” while a BMI of 30 or more classifies the subject as “obese”.

It is evident that any of the compositions and dosage units may suitably contain one or more pharmaceutically acceptable excipients. The term “pharmaceutically acceptable” as used herein is consistent with the art and means compatible with the other ingredients of a pharmaceutical composition and not deleterious to the recipient thereof.

The composition subject of the present invention is particularly suited for formulation as an oral dosage unit, as evidenced by the Examples enclosed herewith which is shown to result in favourable pharmacokinetic properties in hepatically impaired subjects. Preferably, the oral dosage unit for use according to the invention is swallowed. More preferably, the oral dosage unit for use according to the invention is swallowed as a whole. However, equally envisaged are dosage units formulated towards alternative administration methods such as but not limited to sublingual, buccal, or sublabial dosage units.

“Oral dosage unit” encompasses any dosage unit that is intended to and/or suitable for administration to a subject by means of the oral cavity. (Immediate or near-immediate) ingestion of the dosage unit is envisaged but not a limitation for the oral dosage unit of the invention, as detailed further below.

The oral dosage unit described herein may be a solid or semi solid dosage unit such as a tablet, a capsule, a cachet, a pellet, a pill, powder, or granules, or any combination thereof. For example, the oral dosage unit subject of the invention may be a tablet comprising estetrol component-containing granules or a capsule comprising estetrol component-containing granules. The term “solid or semi-solid dosage unit” also encompasses capsules that contain a liquid, e.g. an oil, in which the present estetrol component and/or the optional progestogenic component is dissolved or dispersed.

Tablets and equivalent solid and semi-solid dosage units can suitably contain materials such as binders (e.g. hydroxypropylmethyl cellulose, polyvinyl pyrrolidone (povidone, PVP), other cellulosic materials and starch), diluents (e.g. lactose (monohydrate) and other sugars, starch (e.g. maize starch), dicalcium phosphate and cellulosic materials), disintegrating agents (e.g. starch polymers and cellulosic materials (e.g. sodium starch glycolate) and lubricating agents (e.g., (magnesium) stearates and talc). These tablets and equivalent solid dosage units may be prepared by any suitable means, which have been described in detail in the art (e.g. Kaur, Int Res J Pharm, 2012). Non-limiting examples of processing the estetrol component when manufacturing the dosage unit include wet granulation, e.g. using an aqueous solution or an organic solution, direct compression, 3D printing, or by coating carrier particles with the estetrol component using an organic or inorganic solvent.

As described above, the composition, and hence the (oral) dosage unit may comprise one or more suitable excipients. The term “excipient” as used interchangeably herein and in the art with “carrier” may be indicative for any solvent, diluent, buffer (including but not limited to neutral buffered saline, phosphate buffered saline, or optionally Tris-HCl, acetate or phosphate buffers), solubiliser (including but not limited to Tween 80 or Polysorbate 80), colloid, dispersion medium, vehicle, filler, chelating agent (including but not limited to EDTA or glutathione), amino acid, protein, disintegrant, binder, lubricant, wetting agent, stabiliser, emulsifier, sweetener, colorant, flavoring, aromatiser, thickener, any agent suitable to achieve a depot effect, coating, antifungal agent, any preservative (including but not limited to Thimerosal™, benzalkonium chloride, or benzyl alcohol), antioxidant (including but not limited to ascorbic acid, sodium metabisulfite), tonicity controlling agent, absorption delaying agent, adjuvant, bulking agent (including but not limited to lactose, mannitol) and any other ingredient that may influence any parameter or characteristic of the oral dosage unit subject of the invention. A skilled person understands that one or more excipients may be used in the oral dosage unit on condition that the one or more excipient is compatible with the one or more pharmaceutical ingredient (i.e. in the context of the present invention at least the estetrol component) and that a pharmaceutically acceptable formulation is obtained.

In certain embodiments, the excipient may be an active pharmaceutical ingredient excipient, binder excipient, carrier excipient, co-processed excipient, coating system excipient, controlled release excipient, diluent excipient, disintegrant excipient, dry powder inhalation excipient, effervescent system excipient, emulsifier excipient, lipid excipient, lubricant excipient, modified release excipient, penetration enhancer excipient, permeation enhancer excipient, pH modifier excipient, plasticiser excipient, preservative excipient, preservative excipient, solubiliser excipient, solvent excipient, sustained release excipient, sweetener excipient, taste making excipient, thickener excipient, viscosity modifier excipient, filler excipient, compaction excipient, dry granulation excipient, hot melt extrusion excipient, wet granulation excipient, rapid release agent excipient, increased bioavailability excipient, dispersion excipient, solubility enhancement excipient, stabilizer excipient, capsule filling excipient, or any combination hereof. A skilled person is aware that use of such media and agents for pharmaceutical active substances is common practice and incorporation of these excipients is hence well known in the art. It is evident that all of the used ingredients should be non-toxic in the concentration contained in the final pharmaceutical composition and should not negatively interfere with the activity of the one or more pharmaceutically active ingredients, in this context at least the estetrol component.

Optionally, the composition is comprised in a tablet and comprises next to estetrol excipients fulfilling the functions of a first or a further filler, superdisintegrant, binder, disintegrant, and lubricant. An excipient can perform several of these functions. So can a binder also be a disintegrant. A tablet can also contain two or more excipients that perform the same function, such as two different binders. In preferred embodiments, the composition is comprised in a tablet comprising estetrol, lactose, sodium starch glycolate, maize/corn starch, povidone, and magnesium stearate. Preferably, the composition is comprised in a tablet comprising estetrol monohydrate, lactose monohydrate, sodium starch glycolate type A, maize/corn starch, povidone K30, and magnesium stearate. Optionally, the tablet is coated with a coating agent. In further optional embodiments, the coating agent comprises hypromellose, hydroxypropylcellulose, titanium dioxide, red iron oxide, hydrogenated cottonseed oil, and talc. By means of illustration and not limitation, suitable coating agents are AquaPolish orange 034.23 MS, AquaPolish P blue 064.65 MS, AquaPolish yellow 024.15 MS or AquaPolish pink 044.08 MS. The skilled person is aware that such coatings may be used in combination with a suitable amount of purified water. A skilled person further appreciates that any excipients present in any dosage unit such as an oral dosage unit should adhere to pharmaceutical grade industry quality standards such as Ph. Eur. and USP-NF.

The oral dosage unit may be suitable or even specifically manufactured for sublingual, buccal, and/or sublabial administration. In such embodiments, the solid dosage unit is able to rapidly release the estetrol component when contacted with an aqueous solvent such as saliva. Hence, in these embodiments the solid dosage unit is an orodispersible dosage unit which releases at least about 50%, preferably at least about 60%, more preferably at least about 70%, yet more preferably at least about 80%, most preferably more than about 80% of the estetrol component within about 5 minutes, preferably within about 3 minutes, more preferably within about 2.5 minutes, more preferably within about 90 seconds, most preferably within about 90 seconds. The oral dosage unit may be an orodispersible dosage unit. In such embodiments, the dosage unit rapidly disintegrates in the oral cavity when it comes into contact with saliva and to disperse the estetrol component into the saliva so it may be absorbed through the mucosal lining of the oral cavity. A skilled person is aware of methods to determine the release rate of an estetrol component from a dosage unit. Non-limiting standardized tests generally accepted in the field include the disintegration test according to Ph. Eur. 2.9.1 (“Disintegration of tablets and capsules”) and USP <701> (“Disintegration”), for example using water as the disintegration medium.

The term “sublingual” as used herein refers to the pharmacological route of administration by which the estetrol component diffuses into the blood through tissues under the tongue.

The term “buccal” as used herein refers to the pharmacological route of administration by which the estetrol component diffuses into the blood through tissues of the buccal vestibule, the area inside the mouth between the lining of cheek (the buccal mucosa) and the teeth/gums.

The term “sublabial” as used herein refers to the pharmacological route of administration by which the estetrol component is placed between the lip and the gingiva.

In certain embodiments, the estetrol component is comprised in an immediate release dosage unit or composition.

In certain embodiments, the estetrol component is formulated into a solid dosage unit, including but not limited to hard capsules, soft capsules, tablets, coated tablets such as lacquered tablets or sugar-coated tablets, granules, aqueous or oily solutions, syrups, emulsions, suspensions, ointments, pastes, lotions, gels, inhalants or suppositories. In embodiments wherein the effective amount of an estetrol component is administered by means of oral administration, the oral dosage unit according to the invention is preferably a solid or semi-solid dosage unit such as tablets, capsules, cachets, pellets, pills, powders and granules. The term “solid or semi-solid dosage unit” also encompasses capsules that contain a liquid, e.g. an oil, in which the present estetrol component and/or the optional progestogenic component is dissolved or dispersed. Tablets and equivalent solid and semi-solid dosage units can suitably contain materials such as binders (e.g. hydroxypropylmethyl cellulose, polyvinyl pyrrolidone, other cellulosic materials and starch), diluents (e.g. lactose and other sugars, starch, dicalcium phosphate and cellulosic materials), disintegrating agents (e.g. starch polymers and cellulosic materials) and lubricating agents (e.g., stearates and talc). These tablets and equivalent solid dosage units may be prepared by any suitable means, which have been described in detail in the art (e.g. Kaur, Int Res J Pharm, 2012). Non-limiting examples of processing the estetrol component when manufacturing the dosage unit include wet granulation, e.g. using an aqueous solution or an organic solution, direct compression, 3D printing, or by coating carrier particles with the estetrol component using an organic or inorganic solvent.

By means of illustration and not limitation, an oral dosage unit comprising the composition subject of the present disclosure may be manufactured by a process involving wet granulation. A skilled person appreciates that a wet granulation process may suitable comprise the successive steps of: dispensing and sieving of the active ingredient(s) and excipients, blending the sieved materials in a processor, granulation, screening (i.e. further sieving) of the granules, and one or more blending steps of the sieved granules with one or more further excipients. Afterwards, if desired in view of the final dosage unit the granules may be compressed into for example a tablet, optionally involving a coating step of the tablets.

As discussed above, the composition described herein unexpectedly provides a favourable pharmacokinetic profile in hepatically impaired female subjects. The composition is particularly suited for oral use in alleviating estrogen deficiency symptoms (such as but not limited to menopause-associated symptoms) in this subject population. The favourable pharmacokinetics profile encompasses a multitude of parameters including but not limited to geometric mean (GM) plasma concentration, Cmax, AUCinf, and GM T½ for estetrol. In addition, metabolites of estetrol also display the favourable pharmacokinetic properties, which is reflected for example by the AUCinf for E4-3-glucuronide. “E4-3-glucuronide” interchangeably indicated by the full length name “estetrol-3-glucuronide” is a known and well-documented metabolite of estetrol.

A skilled person is aware of the commonly accepted meaning of the term “geometric mean” and consequently appreciates that the geometric mean indicates the typical value of a set of numbers by using the product of their values

$$( i . e . \sqrt[n]{x 1 \cdot x 2 … \cdot xn} ) .$$

It is understood that when the abbreviation “AUC” is used herein, that this refers to “Area Under the Curve” and is to be interpreted as it common meaning in the art, i.e. the definite integral of a curve that describes the variation of a drug concentration in blood plasma as a function of time. AUC0-24 as used herein expresses the AUC from a time “0”, being the point of administration of the COC to a subject, to a time point of 24 hours. In the context used herein, “AUC” may be interpreted as bioavailability. In accordance, AUCinf indicates the total AUC (time point “0” to infinite). “Cmax” as referred to herein is the maximal or peak blood plasma concentration that is reached of the drug, for example drospirenone. Unless stated otherwise, the AUC and Cmax values may be measured by radioimmunoassays, and/or HPLC and LC MS/MS, which are assays known to a person skilled in the art (for example in Jaffe, Methods of Hormone Radioimmunoassay, 2^{nd} edition, Academic press, 1979, and Chen and Hsu, Development of a LC-MS/MS-based method for determining metolazone concentrations in human plasma: Application to a pharmacokinetic study, J of Food and Drug Anal, 2013). “T½” as used herein refers to the amount of time required for the drug concentration measured in plasma (or other biological matrices) to be reduced to exactly half of its starting concentration or amount.

In the following embodiments, certain parameters defining a particular pharmacokinetic profile are discussed. It is to be understood that these parameters are defined in a relative manner, and are expressed as relative values and/or ranges vis-à-vis a subject with a normal hepatic function. It is evident that “a subject with a normal hepatic function” in the context of the present invention relates to a subject that generally corresponds to the hepatically impaired subject (e.g. age, menopausal status, ethnicity, BMI, etc.) and substantially only differs from said hepatically impaired subject by the absence of a hepatic impairment. It is within the capacities of a medical practitioner to classify a subject to have a normal hepatic function or to have a hepatic impairment by making use of any means or methods deemed necessary by said practitioner such as but not limited to one or more biomarkers. Optionally, a subject having a normal hepatic function in the context of the present invention may be defined as a subject having a Child-Pugh score of less than 5, preferably less than 4, more preferably less than 3, yet more preferably less than 2, most preferably as a subject having a Child-Pugh score of 1. Optionally, the subject having a normal hepatic function in the context of the present invention may be defined as a subject having a Child-Pugh score of from 1 up to and including 4, preferably a Child-Pugh score of from 1 up to and including 3, more preferably a Child-Pugh score of from 1 up to and including 2.

Therefore, in certain embodiments, the composition formulated as an oral dosage unit provides a pharmacokinetic profile in a hepatically impaired subject upon single administration that is characterized by a geometric mean Cmax for estetrol which is less than 3 fold the corresponding GM Cmax in a subject having a normal hepatic function. Preferably, a GM Cmax for estetrol is provided in a hepatically impaired subject which is 2.5 fold or less, more preferably 3 fold or less, even more preferably 1.7 fold or less of the corresponding GM Cmax in a subject having a normal hepatic function. Optionally, the hepatically impaired subject is a subject characterised by a mild hepatic impairment, moderate hepatic impairment, or severe hepatic impairment as defined by the Child-Pugh score. In further embodiments, the composition formulated as an oral dosage unit provides a pharmacokinetic profile in a mild hepatically impaired subject upon single administration that is characterised by a GM Cmax for estetrol which is from about 1.5 fold to about 2.0 fold, preferably about 1.7 fold the corresponding GM Cmax in a subject having a normal hepatic function. In alternative further embodiments, the composition formulated as an oral dosage unit provides a pharmacokinetic profile in a moderate hepatically impaired subject upon single administration that is characterised by a GM Cmax for estetrol which is from about 1.8 fold to about 2.5 fold, preferably about 1.9 fold the corresponding GM Cmax in a subject having a normal hepatic function. In yet alternative further embodiments, the composition formulated as an oral dosage unit provides a pharmacokinetic profile in a severe hepatically impaired subject upon single administration that is characterised by a GM Cmax for estetrol which is from about 5 fold to about 6 fold, preferably about 4.5 fold the corresponding GM Cmax in a subject having a normal hepatic function.

A further favourable pharmacokinetic parameter upon single administration of the composition formulated as an oral dosage unit to a hepatically impaired subject is the absence, or general absence of a difference in peak exposure levels for the estetrol metabolite E4-3-glucuronide. Hence, optionally the composition is characterised by a peak exposure level of E4-3-glucuronide that does not significantly differ from the peak exposure level of E4-3-glucuronide upon single administration of the composition formulated as an oral dosage unit to a subject having a normal hepatic function. In further embodiments, the peak exposure level of E4-3-glucuronide in the hepatically impaired subject is from about 90% to about 110% of the peak exposure level of E4-3-glucuronide in a subject having a normal hepatic function. Preferably, the peak exposure level of E4-3-glucuronide in the hepatically impaired subject is from about 95% to about 105%, preferably from about 97% to about 102.5% of the peak exposure level of E4-3-glucuronide in a subject having a normal hepatic function.

Another favourable pharmacokinetic parameter upon single administration of the composition formulated as an oral dosage unit to a hepatically impaired subject is a GM AUCinf for estetrol and/or E4-3-glucuronide which does not significantly differ from (i.e. is similar to) the GM AUCinf of estetrol and/or E4-3-glucuronide upon single administration of the composition formulated as an oral dosage unit to a subject having a normal hepatic function. Optionally, the GM AUCinf of estetrol and/or E4-3-glucuronide in a mild or moderate hepatically impaired subject is from about 90% to about 110% of the GM AUCinf of estetrol and/or E4-3-glucuronide in a subject having a normal hepatic function. Preferably, the GM AUCinf of estetrol and/or E4-3-glucuronide in the mild or moderate hepatically impaired subject is about 1.1 fold or less of the GM AUCinf of estetrol and/or E4-3-glucuronide in a subject having a normal hepatic function. Preferably the GM AUCinf of estetrol and/or E4-3-glucuronide in a severe hepatically impaired subject is about 2 fold or less of the GM AUCinf of estetrol and/or E4-3-glucuronide in a subject having a normal hepatic function.

Yet a further favourable pharmacokinetic parameter upon single administration of the composition formulated as an oral dosage unit to a hepatically impaired subject is a GM T½ for estetrol and/or metabolites such as E4-3-glucuronide that is similar when compared to the respective GM T½ upon single administration of the composition as an oral dosage unit to a subject having a normal hepatic function. Preferably, the GM T½ for estetrol and/or metabolites such as E4-3-glucuronide in a mild hepatically impaired subject is similar when compared to the respective GM T½ upon single administration of the composition as an oral dosage unit to a subject having a normal hepatic function.

In combination with one or more of the favourable pharmacokinetic profile parameters described herein, the number, frequency, and/or severity of adverse effects (AEs) do not differ in a population of hepatically impaired subjects and a population of subjects having a normal hepatic function. In further embodiments, the number, frequency, and/or severity of treatment-emergent adverse effects (TEAEs) do not differ in a population of hepatically impaired subjects and a population of subjects having a normal hepatic function. It is to be appreciated that treatment-emergent adverse effects are those adverse events occurring from time point of first ingestion of the study until last visit or any event already present that worsens in either intensity or frequency following exposure to the treatment. TEAEs hence encompass newly emergent adverse events due to treatment, but also worsening of pre-existing adverse events due to treatment with the composition described herein. The (treatment-emergent) adverse effects are not particularly limited in the context of the present invention and include by means of illustration and not limitation bloating, breast tenderness, breast swelling, swelling in/of other parts of the body, general feeling of sickness, leg cramps, headaches, indigestion, diarrhoea, and vaginal bleeding.

Remarkably, upon use of the composition subject of the invention the number, frequency, and/or severity of adverse effects do not differ between a population of hepatically impaired subjects and a population of subjects having a normal hepatic function. The composition described herein is therefore safe and well-tolerated in subjects characterised by both normal hepatic function, mild hepatic impairment, moderate hepatic impairments, and severe hepatic impairments. Optionally, the risk for a subject to experience one or more treatment emergent adverse events does not differ between a population of hepatically impaired subjects and a population of subjects having a normal hepatic function. Optionally, the subject is a subject having a hepatic impairment that is not considered a severe hepatic impairment (i.e. a subject that has a mild or moderate hepatic impairment).

As detailed above, the composition which is subject of the present invention comprises an estetrol component equivalent to a daily amount of from about 15 mg to about 25 mg of estetrol. Evidently, this includes embodiments wherein the composition comprises from about 15 mg to about 25 of the estetrol component (a skilled person appreciates that this is encompassed by the definition of “equivalent to from about 15 mg to about 25 mg of estetrol”). In further embodiments, the composition comprises from about 15 mg to about 20 mg of estetrol component, preferably wherein the estetrol component is estetrol, estetrol monohydrate, or an ester of estetrol. Preferably the composition comprises from about 15 mg to about 25 mg of estetrol monohydrate, more preferably from about 15 mg to about 20 mg estetrol monohydrate. In alternative embodiments, the composition comprises from about 15 mg to about 25 mg of estetrol or an ester thereof, preferably from about 15 mg to about 20 mg of estetrol or an ester thereof.

Optionally, the composition comprises about 17 mg of estetrol component, or an amount of estetrol component equivalent to a daily dose of about 17 mg of estetrol component. In further embodiments, the composition comprises about 17 mg of estetrol monohydrate, or an amount of estetrol component equivalent to a daily dose of about 17 mg of estetrol monohydrate. In alternative further embodiments, the composition comprises about 17 mg of estetrol or ester thereof, or an amount of estetrol component equivalent to a daily dose of about 17 mg of estetrol or an estetrol ester.

In alternative embodiments, the composition comprises from about 12 mg to about 28 mg of an estetrol component or an amount of estetrol component equivalent to a daily dose of from about 12 mg to about 28 mg of estetrol. In preferred alternative embodiments, the composition comprises from about 12 mg to about 28 mg of estetrol monohydrate, or an amount of estetrol component equivalent to a daily dose of from about 12 mg to about 28 mg of estetrol monohydrate. In yet alternative embodiments, the composition comprises from about 12 mg to about 28 mg of estetrol or an ester thereof, or an amount of estetrol component equivalent to a daily dose of from about 12 mg to about 28 mg of estetrol or an ester thereof.

Optionally, the composition comprises about 15 mg of estetrol component, or an amount equivalent to about 15 mg of estetrol. Optionally, the composition may comprise about 15 mg of estetrol. In further optional embodiments the composition comprises about 15 mg of estetrol monohydrate.

Alternatively, the composition comprises about 20 mg of estetrol component, or an amount equivalent to about 20 mg of estetrol. Optionally, the composition may comprise about 20 mg of estetrol. In further optional embodiments the composition comprises about 20 mg of estetrol monohydrate.

The estetrol component may be comprised as a multitude of particles in the composition. The particle size of the estetrol component is not particularly limiting. By means of illustration and not limitation, suitable particle sizes may be expressed by means of particle-size distribution values such as but not limited to D(10), D(50), and D(90). A skilled person is well aware how to interpret these parameters. “Particle-size distribution”, commonly abbreviated as “PSD” is a numerical value expressing a relative amount of particles according to size, wherein the relative amount of particles is preferable expressed by mass. For example, the D10 or Dv(10) value signifies the point in the size distribution, up to and including which, 10% of the total volume of the sample (i.e. a collection of particles) is contained. For example, a D10 of 10 μm means that 10% of the sample has a size of maximum 10 μm. The D10, D50, and D90 values are routinely used in the art to calculate the span of a sample, which indicates the width of the size distribution. The span is calculated according to the following formula: (D90−D10)/D50. A skilled person appreciates that representative particle-size distribution values can only be derived from a representative sample.

Optionally, the estetrol particles have a D(10) from about 0.5 μm to about 10 μm, preferably from about 1 μm to about 5 μm, more preferably of from about 1.5 μm to about 2.5 μm. Optionally, the estetrol particles have a D(50) of less than 20 μm, preferably less than 12 μm, more preferably from about 5 μm to about 15 μm, preferably from about 6 μm to about 12 μm, more preferably from about 7 μm to about 11 μm, most preferably from about 8 μm to about 12 μm. Optionally, the estetrol particles have a D(90) from about 15 μm to about 50 μm, preferably from about 20 μm to about 30 μm, more preferably from about 22 μm to about 28 μm.

A multitude of measurement techniques are available for determining particle-size distribution values and include sieve analysis, air elutriation analysis, photo analysis, optical counting, electro resistance counting, sedimentation, laser diffraction, laser obscuration, time of transition, acoustic spectroscopy, ultrasound attenuation microscopy, by means of a cascade impactor, or any combination thereof. Unless explicitly mentioned otherwise, the particle-size distribution values of the present disclosure are obtained by laser diffraction analysis. Laser diffraction analysis, interchangeably annotated in the art by laser diffraction spectroscopy, is a particle measurement technology based on interpretation of laser diffraction patterns passed through an object. Laser diffraction is capable to measure the geometrical dimensions of a particle. Laser diffraction protocols have been described in detail in the art on numerous occasions (e.g. as reviewed in detail in a context of particle analysis in Eshel et al., Soil Science Society of America Journal, 2004).

Optionally, the estetrol particles are further granulated into larger granulates. In certain embodiments, the estetrol component is comprised in the composition as a multitude of larger granulates that have a volume median diameter from about 100 μm to about 4000 μm, preferably from about 200 μm to about 1000 μm, more preferably from about 200 μm to about 600 μm.

In certain embodiments, the estetrol component is the sole (i.e. single, only) pharmaceutically active ingredient part of the composition. The term “pharmaceutically active ingredient”, interchangeably used throughout the present disclosure with “pharmaceutically active agent” is to be interpreted according to the definition of the term by the World Health organisation: “a substance used in a finished pharmaceutical product (FPP), intended to display pharmacological activity or to otherwise have direct effect in the diagnosis, cure, mitigation, treatment or prevention of disease, or to have direct effect in restoring, correcting or modifying physiological functions in human beings”. More particularly, in certain embodiments no progestogenic component is co-administered with the estetrol component (neither in the same composition or oral dosage unit nor in a further composition or dosage unit that is co-administered). In embodiments wherein the hepatically impaired subject underwent hysterectomy, the estetrol component is preferably administered as sole pharmaceutically active ingredient.

In alternative embodiments, the composition comprises at least one further pharmaceutically active ingredient in addition to the estetrol component.

Optionally, the oral dosage form comprises as a further pharmaceutically active ingredient a progestogenic component, or the method of treatment comprises a step of co-administering a progestogenic component to the subject. While embodiments wherein the progestogenic component is comprised in the same composition as the estetrol component are preferred, embodiments wherein the progestogenic component is administered by means of a separate composition or dosage unit are equally envisaged.

The terms “progestogen”, “gestagen”, or “gestogen” and derived hereof “progestogenic components” as used both herein and in the art refer to any molecule that produces effects similar to those of the natural female sex hormone progesterone in the body of a subject. Progestogens are considered to be agonists of the progesterone receptors and their functions have been thoroughly examined in the art (inter alia discussed in Kuhl, Climacteric, 2005). Progestins are a subgroup of progestogens that comprise synthetic progestogens. While the above terms may be used interchangeably in the art, there is a general understanding that when progestin is mentioned, synthetic progestogens are meant.

Examples of progestogenic components envisaged by the invention include without limitation: levonorgestrel, norgestimate, norethisterone, dydrogesterone, drospirenone, 3-beta-hydroxydesogestrel, 3-ketodesogestrel, 17-deacetylnorgestimate, 19-norprogesterone, acetoxypregnenolone, allylestrenol, amgestone, chlormadinone, cyproterone, demegestone, desogestrel, dienogest, dihydrogesterone, dimethisterone, ethisterone, ethynodiol diacetate, fluorogestone acetate, gastrinone, gestodene, gestrinone, hydroxymethylprogesterone, hydroxyprogesterone, lynestrenol, mecirogestone, medroxyprogesterone, megestrol, melengestrol, nomegestrol, norethindrone, norethynodrel, norgestrel (including d-norgestrel, and dl-norgestrel), norgestrienone, normethisterone, progesterone, quingestanol, (17α)-17-hydroxy-11-methylene-19-norpregna-4, 15-dien-20-yn-3-one, tibolone, trimegestone, algestone-acetophenide, nestorone, promegestone, 17-hydroxyprogesterone esters, 19-nor-17hydroxyprogesterone, 17alpha-ethynyltestosterone, 17alpha-ethynil-19-nortestosterone, d-17beta-acetoxy-13beta-ethyl-17alpha-ethynylgon-4-en-3-one oxime, 6beta, 7beta; 15beta, 16beta-dimethylene-3-oxo-17-pregna-4,9(11)-diene-21, 17beta-carbolactone or tanaproget and precursors of these components that are capable of liberating these progestogens in vivo.

The progestogenic components may be selected from the group comprising: progesterone, drospirenone, norethisterone, norethisteron-acetate (NETA), dydrogesterone, levonorgestrel (LNG), etonogestrel, norgestrel, nomegestrol, nomegestrol-acetate (NOMAC), trimegestone, nestorone, dydrogesterone, gestodene, desogestrel, norgestimate, cyproterone acetate, dienogest, and chlormadinone. Particularly preferred progestogens in the present context include without limitation drospirenone, progesterone and dydrogesterone.

In certain embodiments, the progestogen is a naturally occurring progestogen. In alternative embodiments, the progestogen is a progestin.

“Drospirenone” (abbreviated as DRSP, PubChem CID: 68873) is an example of a progestogenic component and enjoys a widespread use in Combined Oral Contraceptives (commonly abbreviated as COCs) due to its antimineralocorticoid and antiandrogenic activity combined with a general low off-target activity. In general, drospirenone-containing COCs are referred to as fourth generation COCs. Non-limiting examples of commercially available COCs comprising drospirenone are known as “Yaz®” and “Yasmin®”. An illustrative example of a drospirenone only progestogen pill is “Slynd®”, which is also commercially available. Additionally, hormone replacement therapy compositions comprising an estrogen such as estradiol and drospirenone are available such as “Angeliq®”. Drospirenone may alternatively be indicated in the art by its molecular formula C₂₄H₃₀O₃, or by the structural formula (III):

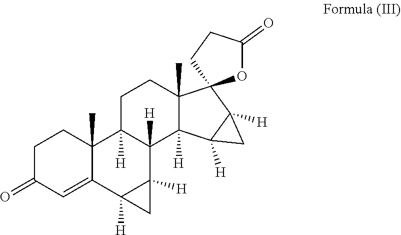

It is understood that when the term “drospirenone” is used herein, any drospirenone derivatives are also envisaged.

The methods described herein may include the administration of drospirenone to the subject receiving the estetrol component. In certain embodiments, the composition may comprise from about 0.25 mg to about 10 mg drospirenone, or a progestogenic component in an amount equivalent to a daily dose from about 0.25 mg to about 10 mg drospirenone. Preferably, the composition may comprise from about 1 mg to about 4 mg drospirenone or a progestogenic component in an amount equivalent to a daily dose from about 1 mg to about 4 mg drospirenone. More preferably, the composition may comprise from about 1 mg to about 3 mg of drospirenone, or a progestogenic component in an amount equivalent to a daily dose from about 1 mg to about 3 mg drospirenone. Yet more preferably, the composition may comprise from about 2.5 mg to about 3.5 mg of drospirenone, or a progestogenic component in an amount equivalent to a daily dose of from about 2.5 to about 3.5 mg of drospirenone.

Optionally, the composition comprises an estetrol component, preferably estetrol monohydrate, in an amount from about 15 mg to about 25 mg and drospirenone in an amount of from about 0.25 mg to about 10 mg. In further optional embodiments, the composition comprises from about 15 mg to about 20 mg of an estetrol component, preferably estetrol monohydrate, and from about 1 mg to about 4 mg drospirenone. In yet further optional embodiments, the composition comprises about 15 mg or about 20 mg of an estetrol component, preferably estetrol monohydrate, and about 3 mg of drospirenone.

“Progesterone” (commonly abbreviated as “P4”; PubChem CID 5994) is an endogenous steroid and progestogen sex hormone involved in the menstrual cycle, pregnancy, and embryogenesis of women and constitutes the major progestogen in the body. Progesterone is a well-documented substance and has been used in the art for indications including but not limited to contraception, female hormone replacement therapy, and feminizing hormone therapy. Progesterone may be indicated in the art by reference to its structural formula C₂₁H₃₀O₂, or by the structural formula (IV):

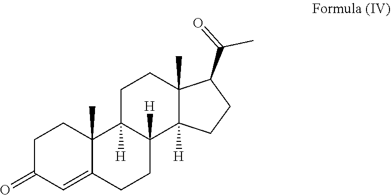

It is understood that when the term “progesterone” is used herein, any progesterone derivatives are also envisaged.

In certain embodiments, the composition may comprise from about 10 mg to about 500 mg, preferably from about 25 mg to about 300 mg progesterone, or a progestogenic component in an amount equivalent to a daily dose from about 10 mg to about 500 mg, preferably from about 25 mg to about 300 mg progesterone. Preferably, the composition may comprise from about 100 mg to about 200 mg progesterone or a progestogenic component in an amount equivalent to a daily dose from about 100 mg to about 200 mg progesterone.

Optionally, the composition comprises an estetrol component, preferably estetrol monohydrate, in an amount from about 15 mg to about 25 mg and progesterone in an amount of from about 25 mg to about 300 mg. In further optional embodiments, the composition comprises from about 15 mg to about 20 mg of an estetrol component, preferably estetrol monohydrate, and from about 100 mg to about 200 mg progesterone. In yet further optional embodiments, the composition comprises about 15 mg or about 20 mg of an estetrol component, preferably estetrol monohydrate, and from about 100 mg to about 200 mg of progesterone. In another embodiment, progesterone is used at a daily dose of 100 mg to 200 mg when it is used sequentially, for example when it is administered during about 14 days every month. In yet another embodiment, progesterone is used at a daily dose of 100 mg to 200 mg when it is used sequentially, for example when it is administered during about 14 days after the estetrol component has been administered orally once daily at a dose of 15 or 20 mg for a minimum of 12 weeks and no longer than 13 weeks. Preferably, progesterone is used once daily for 14 consecutive days after completion of treatment with the estetrol component.

The progestin “dydrogesterone” (PubChem CID 9051), interchangeably indicated in the art such as “isopregnenone” and “didrogesteron”, has been used for numerous medical indications including dysfunctional bleeding, infertility, dysmenorrhea, endometriosis, and menopause hormone therapy. Dydrogesterone may be indicated in the art by reference to its structural formula C₂₁H₂₈O₂ or by the structural formula (V):

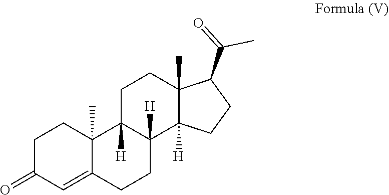

It is understood that when the term “dydrogesterone” is used herein, any dydrogesterone derivatives are also envisaged.

In certain embodiments, the composition may comprise from about 1 mg to about 20 mg dydrogesterone, or a progestogenic component in an amount equivalent to a daily dose from about 5 mg to about 10 mg dydrogesterone. Preferably, the composition may comprise from about 1 mg to about 20 mg dydrogesterone or a progestogenic component in an amount equivalent to a daily dose from about 5 mg to about 10 mg dydrogesterone.

Optionally, the composition comprises an estetrol component, preferably estetrol monohydrate, in an amount from about 15 mg to about 25 mg and dydrogesterone in an amount of from about 1 mg to about 20 mg. In further optional embodiments, the composition comprises from about 15 mg to about 20 mg of an estetrol component, preferably estetrol monohydrate, and from about 5 mg to about 10 mg dydrogesterone. In yet further optional embodiments, the composition comprises about 15 mg or about 20 mg of an estetrol component, preferably estetrol monohydrate, and from about 5 mg to about 10 mg of dydrogesterone.

In the context of the present invention, other compounds (i.e. components, agents) may be used in conjunction with the estetrol component for administering to women who have an uterus. Selective Estrogen Receptor Modulators (SERMs) defines a category of such compounds, which are contemplated as useful complements to the estetrol component in the methods of the invention. A preferred SERM for use in the context of the present invention is bazedoxifene. In the methods and compositions further described herein, it has to be understood that when reference is made to a “progestogenic component”, such reference includes SERMs and in particular bazedoxifene. Preferably, the bazedoxifene is administered at a daily dose of from about 10 mg to 50 mg. More preferably, bazedoxifene is administered at a daily dose of from about 15 to about 25 mg. Most preferably, bazedoxifene is administered at a daily dose of about 20 mg.

The composition may be formulated into a dosage unit that is to be administered to a subject using any time interval deemed appropriate by a skilled person. A preferred administration scheme in the context of the present invention is a daily administration scheme (i.e. one administration every about 24 hours). In such embodiments, the composition described herein is therefore representative of a daily composition, a daily dosage unit. In alternative embodiments, the composition is formulated for a multiple dose per day administration scheme, which then accumulates to achieve the dose in line with the present invention, e.g. 15 to 25 mg of the estetrol component.

Optionally, the dosage unit is administered to a subject by means of a continuous administration schedule. The terms “continuous” and “continuously” as used herein, means that the dosage units are administered at relatively regular intervals, with no (therapeutically) significant interruptions. Naturally, minor interruptions may occur that do not affect the overall effectiveness of the present method, and indeed such aberrations are encompassed by the present invention. In a preferred embodiment, and more arithmetically, the administration regimen is deemed to be continuous if the longest interval between two subsequent administrations is not more than 3.5 times as long as the average interval. Even more preferably said longest interval is not more than 2.5 times, most preferably not more than 1.5 times as long as the average interval. By means of illustration and not limitation, the treatment strategies and method of treatments described herein preferably employ continuous administration of the estetrol component during a period of at least 10 days, preferably of at least 20 days.

Alternatively, the dosage unit is administered to a subject by means of a sequential administration schedule. It is to be appreciated that the term “sequential” means an administration during, for example, 10 to 14 days each month or during 14 days every 3 months. Sequential administration schedules are particularly envisaged wherein a progestogenic component is part of the composition or treatment described herein.

A further aspect of the invention is directed to packaging units comprising the dosage units described herein. The packaging units may comprise at least 14, preferably at least 21, even more preferably at least 28, containers for holding separately packaged and individually removable dosage units, wherein each container comprises at least one dosage unit comprising of from about 15 to about 25 mg of an estetrol component. Preferably, the separately packaged and individually removable dosage units are oral dosage units. More preferably, each of the separately packaged and individually removable dosage units comprise about 15 or about 20 mg of estetrol component, preferably about 15 mg or 20 mg of estetrol monohydrate.

Optionally, the packaging units additionally comprise at least 10, preferably 12, more preferably 14, additional containers for holding separately packaged and individually removable dosage units, wherein each additional container comprises at least one dosage unit comprising a progestogenic component. Optionally, each of the additional containers for holding the dosage units comprising the progestogenic component are individually visually arranged next to a container holding a dosage unit comprising the estetrol component when these two dosage units have to be administered on the same day. Optionally, the packaging unit additionally comprises the same number of additional containers for holding separately packaged and individually removable oral dosage forms, wherein each additional container comprises at least one daily, preferably solid, oral dosage form comprising a progestogen, preferably wherein said progestogen is selected from drospirenone, progesterone and dydrogesterone.

A skilled person will understand that the embodiments described above which are directed to packaging units may equivalently be presented as a kit-of-parts containing a first packaging unit, e.g. a blister pack, containing the daily oral dosage units comprising the estetrol component, and a second, distinct, packaging unit, e.g. a second, distinct, blister pack, containing the daily oral dosage units comprising the progestogen.

The skilled person will additionally know that, within the scope of the present invention, each packaging unit, e.g. blister pack, may be numbered or otherwise marked.

The packaging units may be provided in any suitable packaging means known in the art, non-limiting examples being troches, sachets, pouches, bottles, films, sprays, microcapsules, implants, rods or blister packs.

By means of illustration and not limitation, each packaging unit may be a sealed blister pack with a cardboard, paperboard, foil plastic backing and enclosed in a suitable cover. Also envisaged in any one of the aspects defined herein are packaging units such as bottles. The material of the bottle is not particularly limiting. In preferred embodiments, the bottle is a glass bottle characterised by a colour capable of reducing or preventing degradation of the contents of the bottle by e.g. UV light while maintaining a degree of transparency that allows for visual inspection of the contents of said bottle. Suitable colours include without limitation amber, cobalt, or vintage green.

In a particular embodiment of the invention the packaging unit comprises 28 containers or a multiple of 28 containers, such as 2 to 12 multiple of 28 containers.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims. The herein disclosed aspects and embodiments of the invention are further supported by the following non-limiting examples. The following specific experimental examples are provided in support of the claimed invention but are not to be seen as limiting the scope of the invention.

### EXAMPLES

### Example 1. Clinical Study Report (CSR) in Hepatically Impaired Subjects

### 1. Study Objectives

### 1.1 Primary

To assess the PK of E4 in subjects with different categories of hepatic impairment and in subjects with normal hepatic function.

### 1.2 Secondary

To assess the PK of E4 metabolites in subjects with different categories of hepatic impairment and in subjects with normal hepatic function.
To assess the safety of E4 in subjects with different categories of hepatic impairment and in subjects with normal hepatic function.

### 2. Investigational Plan

### 2.1 Overall Study Design and Plan

In the sections below, the study design and plan are summarized. See also the summary of clinical procedures and assessments in Table 2 and Table 3 in Section 2.5.1.

### 2.1.1 Type of Study

This was a Phase 1, multi-centre, open-label, PK, and safety study of a single oral dose of 20 mg E4 monohydrate in female subjects with normal hepatic function (Group 1), mild hepatic impairment (Group 2), moderate hepatic impairment (Group 3), and severe hepatic impairment (Group 4), as defined using the Child-Pugh classification at Screening.

The study consisted of an eligibility Screening period (within 28 days prior to study drug administration), an Assessment Period with in-house period and outpatient visits (including a single oral dose of 20 mg E4 on Day 1), and a follow up visit. All subjects were confined to the study site from Day-1 (the day before dosing) until Day 4 and returned to the site every day from Day 5 to Day 9. Alternatively, the subjects could stay for a prolonged in-house period in agreement with the Investigator. The follow-up visit occurred on Day 14 (+2 days).

### 2.1.2 Screening Period

All subjects were screened within 28 days prior to study drug administration.

Subjects signed the study specific informed consent form (ICF) prior to any study specific screening procedures being performed. The written informed consent was obtained for all subjects, regardless of their eligibility for the study; the signed ICFs were retained and archived at the CRO and another copy was provided to the subject. The hepatic function of subjects with hepatic impairment was assessed according to Child-Pugh classification within the screening period. The results of this assessment were used for the categorization into the groups of subjects with mild, moderate, and severe hepatic impairment, respectively (see Section 2.3). Subjects with hepatic impairment were assigned to these groups based on results that had to be obtained within 15 days prior to dosing. If screening was performed prior to 15 days pre-dose the assessments used for the Child-Pugh classification had to be repeated, and the most recent results were used for the categorization of the respective subject into the corresponding hepatic impairment group. If this assessment was performed on Day-2 the subjects could choose whether to start the confinement period from then on or to attend the unit in an ambulatory fashion.

After signing of the ICF, eligibility screening consisted of the assessments as presented in Table 2.

### 2.1.3 Assessment Period

All subjects arrived at the study site in the morning of Day-1 in a fasted state (no food for at least 8 h, water was allowed) to have the scheduled assessments performed in order to confirm eligibility as outlined in the inclusion and exclusion criteria (Section 2.3.1 and Section 2.3.2, respectively). In the morning of Day 1 following an overnight fast, the subjects received oral dosing of 20 mg E4 monohydrate and remained at the site. Study-related assessments were performed at time points indicated in the Schedule of Assessments in Table 2.

Serial blood sampling for PK assessments started on Day 1 and proceeded until Day 9 according to the Pharmacokinetic Sampling Schedule presented in Table 3.

On Day 4, the subjects could leave the study site and return for outpatient visits from Day 5 to Day 9. Alternatively, and at the discretion of the Investigator, the subjects could remain for an extended in-house period until Day 9.

For a detailed overview of assessments, refer to Section 2.5.1 and the Schedule of Assessments (Table 2).

### 2.1.4 Follow-Up

The follow-up medical examination was performed on Day 14 (+2 days). For a detailed overview of assessments, refer to Section 2.5.1 and the Schedule of Assessments (Table 2).

### 2.2 Discussion of Study Design

The study adhered to the guidelines for studies with hepatic impaired subjects according to the recommendations given by the Food and Drug Administration (FDA) and the European Medicines Agency (EMA). 7,8

In accordance with the objectives, a parallel design was used to explore the potential difference in PK profiles between subjects with different stages of impaired hepatic function and subjects with normal hepatic function. Hepatic function was scored following the Child-Pugh classification as recommended in guidelines from the FDA and the EMA (FDA Guidance for Industry, Pharmacokinetics in Patients with Impaired Hepatic Function, 2003; EMA Guideline on the Evaluation of the Pharmacokinetics of Medicinal Products in Patients with Impaired Hepatic Function, 2005).

Initially, 2 subjects were enrolled in the hepatic impairment groups (Groups 2, 3, and 4). Once those 2 subjects in a specific group had completed Day 5, a safety laboratory data review for that group was performed. Providing the outcome of this data review was satisfactory (see Section 2.8) afterwards, the remaining subjects in that group could be enrolled.

The study was open-label as all subjects received the same treatment and needed to be assigned to the appropriate hepatic function group.

### 2.3 Selection of Study Population

Approximately 32 female subjects were to be enrolled into this study: 24 subjects with hepatic impairment (8 subjects in each severity group: mild, moderate, and severe) and 8 subjects with normal hepatic function who represented the control group. Subjects could be replaced, or additional subjects could be enrolled to ensure a minimum of 6 evaluable subjects per group and a sufficient number of subjects for matching.

The hepatic function was assessed in subjects with hepatic impairment (liver cirrhosis) using the Child-Pugh scoring system (Table 1) and the lab results at Screening.

TABLE 1
Classification of Hepatic Function - Child-Pugh Score

| | 1 point | 2 points | 3 points |
|---|---|---|---|
| Albumin (g/L) | >35 | 35-28 | <28 |
| | (>3.5 g/dL) | (3.5-2.8 g/dL) | (<2.8 g/dL) |
| Total bilirubin (μmol/L) | <34.2 | 34.2-51.3 | >51.3 |
| | (<2 mg/dL) | (2-3 mg/dL) | (>3 mg/dL) |
| Prothrombin time | <4 | 4-6 | >6 |
| prolongation (s) | | | |
| Ascites | Absent | Slight | Moderate to severe |
| Encephalopathy*^{,†} | Absent | Grade 1 or 2 | Grade 3 or 4 |

Interpretation:
Grade A: 5-6 points;
Grade B: 7-9 points;
Grade C: 10-15 points
*Assessment of encephalopathy was primarily based on clinical signs and symptoms as detailed below; an encephalogram was not obligatory for grading of encephalopathy
^{†}Grade 0: normal consciousness, personality, neurological examination, electroencephalogram;
Grade 1: restless, sleep disturbed, irritable/agitated, tremor, impaired handwriting, 5 cps waves;
Grade 2: lethargic, time-disoriented, inappropriate, asterixis, ataxia, slow triphasic waves;
Grade 3: somnolent, stuporous, place-disoriented, hyperactive reflexes, rigidity, slower waves;
Grade 4: unrousable coma, no personality/behavior, decerebrate, slow 2-3 cps delta activity

The hepatic function groups were defined as follows:
Group 1: normal hepatic function
Group 2: mild hepatic impairment (Child-Pugh score 5-6** points)
Group 3: moderate hepatic impairment (Child-Pugh score 7-9* points)
Group 4: severe hepatic impairment (Child-Pugh score 10-14* points)
   ** Subjects with Grade≥2 encephalopathy were not to be enrolled.

The control group (Group 1) was matched with the hepatically impaired population with respect to age, body mass index (BMI), and smoking habit. Subjects with normal hepatic function were enrolled after at least 50% of each group of the hepatic impaired subjects had been dosed, and the median, minimum, and maximum of age and BMI of Groups 2 to 4 were determined. The enrolment of subjects with normal hepatic function occurred within the calculated ranges of age and BMI (extremes covered as far as possible, approximately 50% of subjects on each side of the median). An approximately similar ratio of smokers/non-smokers was enrolled.

### 2.3.1 Inclusion Criteria

The following inclusion criteria had to be met for a subject to be eligible for inclusion in the study.

### 2.3.1.1 All Subjects

1. Able to understand and willing to sign the ICF and able to comply with the study restrictions.
2. Adult female subjects age 18 to 75 years, inclusive, at the time of informed consent.
3. BMI 18.0 to 35.0 kg/m2, inclusive, where BMI (kg/m2)=body weight (kg)/height2 (m2).
4. Subjects could be enrolled if they were:
   a. documented to be surgically sterile or postmenopausal (amenorrhea>1 year and FSH≥30 mU/mL), or
   b. having a negative serum pregnancy test at Screening and negative urine pregnancy test at Day-1, and willing to use a non-hormonal method of contraception (i.e., non-hormonal intrauterine device, vasectomized partner, sexual abstinence, male or female condom with or without spermicide, cap, diaphragm or sponge with spermicide) for the duration of the study (Screening to Follow-up).
5. Willing and able to sign the ICF.
6. Willing and able to comply with the study procedures.

### 2.3.1.2 Hepatically Impaired Subjects (in Addition)

7. Diagnosis of cirrhosis due to parenchymal liver disease, which was confirmed and documented by at least one of the following: hepatic ultrasound, computed axial tomography (CT) scan, magnetic resonance imaging (MRI), and/or liver biopsy.
8. Stable hepatic impairment, defined as no clinically significant change in disease status for 1 month, as judged by the Investigator.
   2.3.1.3 Subjects with Normal Hepatic Function (in Addition)
9. Subjects with absence of hepatic diseases and with normal hepatic function as judged by the Investigator.
10. Judged to be in good physical and mental health in the opinion of the Investigator on the basis of a medical history and medical evaluation that reveals the absence of any clinically relevant abnormality (including a physical examination, medical history, vital signs [systolic blood pressure≥100 mmHg and ≤140 mmHg, diastolic blood pressure>60 mmHg and ≤90 mmHg], and the results of biochemistry, coagulation and haematology tests and urinalysis carried out at screening and Day 1).

Subjects with a chronic (>3 months) stable non-oncologic disease under clinical control (e.g, hypertension, diabetes mellitus) may participate.

### 2.3.2 Exclusion Criteria

A subject who met any of the following exclusion criteria was not eligible for inclusion in the study:

### 2.3.2.1 All Subjects

1. Clinically relevant abnormal medical history, abnormal findings on physical examination (including gynaecological examination), vital signs, or laboratory tests at Screening that the Investigator judged as likely to interfere with the objectives of the trial or the safety of the volunteer except for conditions associated with hepatic impairment in subjects with compromised hepatic function (Groups 2 to 4).
2. Surgery (eg, stomach bypass) or medical condition that might significantly affect absorption of medicines (eg, pancreatic injury or pancreatitis, gastritis) as judged by the Investigator.
3. Documented congenital QT syndrome.
4. Unstable ischemic heart disease or severe heart failure (New York Heart Association Class III or IV).
5. Uncontrolled treated/untreated hypertension (defined as a mean of 3 repeated measurements for systolic blood pressure≥180 mmHg and/or diastolic blood pressure≥110 mmHg); current or documented history of repeated clinically significant hypotension or severe episodes of orthostatic hypotension (systolic blood pressure<90 mmHg and/or diastolic blood pressure<50 mmHg).
6. Primary biliary cirrhosis.
7. History of hormone-related (estrogen/progestin) cancer or history of other cancer judged not to be in full remission or presence of cancer (except basal cell skin cancer or squamous cell skin cancer).
8. Acute illness within 14 days prior to study drug administration unless mild in severity and approved by the Investigator and Sponsor's medical representative.
9. Presence of active infection requiring antibiotics.
10. Consumption of Seville oranges, grapefruit or grapefruit juice, pomelo, star fruit, cranberries, or menthol (drinks or food) within 7 days prior to study drug administration until the end of the Assessment Period.
11. Use of any prescription drugs or herbal supplements acting as strong inducers and inhibitors of CYP3A4 functions (e.g., St. John's Wort) within 14 days before Day 1 and until Follow Up. For treatment of pain, such as headaches or musculoskeletal pain, acetaminophen as first-line, ibuprofen, indomethacin, and metamizole with pitofenone were allowed. The dose of the analgesic was determined by the investigator. The Sponsor reserved the right to review any medication chronically used by hepatically impaired subjects in view of their eligibility for the study.
12. Any over-the-counter medication or dietary supplements (vitamins included) within 14 days prior to the study drug administration until study completion.
13. Ingestion of alcohol within 48 h prior to study drug administration and until Day 9. Outside this period, regular alcohol consumption below 17 units per week (1 unit equals 250 mL of beer, 75 mL of wine or 25 mL of spirits) was allowed.
14. Smoking of more than 10 cigarettes (or equivalent nicotine consumption) per day. Tobacco that contains menthol was not allowed to be consumed.
15. Exposure to another investigational drug within 30 days prior to study drug administration (or 5 times the half-life of the drug, whichever was longer) or exposure to more than 3 new investigational agents within 12 months prior to study drug administration. Previous participation in the current study with exposure to E4.
16. Donation or loss of more than 450 mL blood during the 3 months before the start of Screening.
17. Non-hysterectomied female subjects who are pregnant, nursing, or planning to become pregnant during the study.
18. Clinically significant renal disease (creatinine clearance [CLCr]<60 mL/min as calculated by the Cockcroft-Gault formula at Screening, see Table 4).
19. Positive serology for human immunodeficiency virus antibodies (anti-HIV)-1/2 at Screening.
20. History of drug addiction (including soft drugs like cannabis products).
21. Positive urine drug (opiates, methadone, cocaine, amphetamines [including methamphetamines], cannabinoids, barbiturates, benzodiazepines, and tricyclic antidepressants) screen (if not medically indicated and prescribed, except for cannabinoid containing drugs which will not be allowed under any circumstances) or alcohol breath test at Screening and/or Day-1.
22. History of relevant drug and/or food allergies.
23. Legal incapacity or limited legal capacity.
24. Undiagnosed vaginal bleeding in the last 12 months.
25. History of venous or arterial thromboembolic disease (eg, deep vein thrombosis, pulmonary embolism, stroke, myocardial infarction, angina pectoris, etc.) or history of known coagulopathy.
26. Abnormal and clinically significant cervical Pap smear for subjects with cervix (written documentation of prior test within 12 months or test at Screening exam) with evidence of cervical dysplasia. Women with a diagnosis of atypical squamous cells of undetermined significance (ASCUS) were allowed.
27. Subjects who were not in euthyroid condition (hyperthyroidism or hypothyroidism), including subjects who receive drug treatment (eg, L-thyroxine or anti-thyroid drugs).

### 2.3.2.2 Hepatically Impaired Subjects (in Addition)

28. History of oesophageal bleeding within the last 3 months prior to study drug administration.
29. Severe hepatic encephalopathy (Grade≥2) or degree of central nervous system impairment which the Investigator considers sufficiently serious to interfere with the informed consent, the conduct, the completion, or the results of this trial, or constitutes an unacceptable risk to the subject.
30. History of liver transplantation.
31. Advanced ascites and ascites which require emptying and albumin supplementation, as judged by the Investigator.
32. Haemoglobin concentration<105 g/L.
   2.3.2.3 Subjects with Normal Hepatic Function (in Addition)
33. Positive serology for hepatitis B surface antigen (HBsAg) or anti-hepatitis C virus (HCV) unless the absence of an active hepatitis B/C infection was confirmed by a polymerase chain reaction (PCR) test.
   2.3.3 Removal of Subjects from Assessment

Participation in the study was strictly voluntary. A subject had the right to withdraw from the study at any time for any reason, without any reprisal.

The Investigator had the right to terminate participation of a subject for any of the following reasons: difficulties in obtaining blood samples, violation of the protocol, severe adverse events (AEs) or serious adverse events (SAEs), or for any other reason relating to the subject's safety or integrity of the study data.

If a subject was to be withdrawn from the study, the Sponsor was to be informed immediately. If there was a medical reason for withdrawal, the subject was to remain under the supervision of the Investigator until satisfactory health had returned.

If the Investigator was to withdraw a subject for a study drug related reason (according to the judgment of the Investigator) she was to be considered a dropout. Dropouts were to be replaced, where the Sponsor deemed this necessary.

If a subject did not complete the study for a non-drug related reason, he/she would be considered a non-completer and was to be replaced to ensure a sufficient number of 6 evaluable subjects.

The decision regarding the replacement of subjects was to be documented.

The CRO was to make every effort to ensure that non completers and dropouts who had received study drug completed the safety follow up assessments.

### 2.3.4 Premature Termination of the Study

The procedures for a premature termination of the trial were detailed in the study protocol.

### 2.4 Treatments

### 2.4.1 Treatments Administered

Each enrolled subject was to receive a single oral dose of 20 mg of E4 (estetrol) as monohydrate as a film-coated tablet.

### 2.4.2 Identity of Investigational Product

### Active Medication

Active substance: Estetrol as monohydrate
Activity: Agonist of Estrogen receptor (ER), with a higher affinity for ERα
Indication: not applicable
Dose 20 mg
Strength: 1× 20 mg
Dosage form: Film coated tablets for oral administration

Packaging and labelling was done in accordance with The Rules Governing Medicinal Products in the European Union, Volume 4: Good Manufacturing Practice (GMP). The investigational medicinal product (IMP) labels included all the information required by Annex 13 to GMP.

### 2.4.3 Method of Assigning Subjects to Hepatic Function Groups

After obtaining oral and written informed consent, subjects with hepatic impairment and normal hepatic function were screened according to the inclusion and exclusion criteria. For hepatically impaired subjects, the allocation to one of the 3 hepatic impairment groups was based on the Child-Pugh classification (see Section 2.2). If the hepatic function test was repeated during screening, the most recent result was used for group allocation. Subjects for the control group had to have no hepatic diseases and a normal hepatic function as judged by the Investigator. Subjects who complied with all selection criteria received a unique subject number upon enrolment in the study. Subject numbers were allocated sequentially in the order in which the subjects were enrolled. The Investigator or designee entered the corresponding subject number in each subject's electronic case report form (eCRF). The subject number ensured identification until the follow-up visit.

Subjects dropping-out or withdrawing, for any reason, prior to enrolment, were considered as screen failures. Such subjects did not receive a subject number. The Investigator kept a screening log of all subjects screened in order to assess the numbers and characteristics of the excluded subjects, and the reasons for their exclusion.

### 2.4.4 Selection of Doses in the Study

E4 was well tolerated and safe up to 100 mg given as a single dose and up to 40 mg given once daily for 28 days, and the mass balance study in humans showed that the hepatic elimination contributes approximately 20% to the overall elimination. It was assumed that an oral dose of 20 mg of E4 would provide a sufficient safety margin even if a 5-fold higher exposure was expected in subjects with severe hepatic impairment than in subjects with normal hepatic function.

### 2.4.5 Timing of Doses in the Study

Study drug was administered to subjects in the morning after an overnight fast of at least 8 h.

The study drug was swallowed together with 240 mL tap water (room temperature). The study drug was not to be chewed on. Fasting continued for a period of 4 h after drug administration. During fasting, no fluids were allowed except water; however, water was not allowed from 2 h pre-dose until 1 h post-dose (apart from the water taken with the dose as described above). When not fasting, fluids were allowed ad libitum under consideration of the restrictions listed in Section 2.4.8.

Administration of the study drug was supervised by the Investigator or deputy. After drug administration a mouth and hand inspection took place.

Whenever possible, administration of regular medication was to be avoided 2 h pre- and 2 h post-IMP dosing, with exception of drugs that were prescribed for specific time, or drugs to be administered with meal.

### 2.4.6 Meals During the Study

A fasting period of at least 8 h was required before obtaining clinical laboratory samples at all time points, thus before Screening, admission on Day-1, Day 2, Day 3, Day 5, Day 9, and Follow-up.

With the exception of the restrictions with respect to caffeine- and alcohol containing beverages or food as described in Section 2.4.8, there were no special requirements related to food and beverage intake. When not fasting, meals and snacks were provided according to clinic standards.

### 2.4.7 Blinding

This was an open-label study.

### 2.4.8 Previous and Concomitant Therapy and Other Restrictions During the Study

All medications (prescription and over-the-counter) taken from 30 days before start of Screening until Follow-up were recorded in the appropriate section of the eCRF.

Apart from the restrictions listed in the inclusion and exclusion criteria (Section 2.3), the following restrictions had to be followed:

All subjects:
Ingestion of alcohol was not allowed within 48 h prior to study drug administration until the end of the Assessment Period.
No food or drink containing grapefruit, Seville oranges, pomelos, star fruit or cranberries were allowed from 7 days prior to study drug administration until the end of the Assessment Period.
No food or drink containing caffeine was allowed from 48 h prior to study drug administration until the end of the Assessment Period.
Postmenopausal women had to employ the same contraceptive measures as described for non-hysterectomied women, unless the postmenopausal status was confirmed by FSH test and absence of menses for more than 12 months.
Strenuous activity, sunbathing, or contact sports were not allowed from 96 h (4 days) prior to entry in the clinic research centre until the final follow-up visit.
Smoking was prohibited during the in-house stay.
For treatment of pain, such as headaches or musculoskeletal pain, acetaminophen as first-line, ibuprofen, indomethacin, and metamizole with pitofenone were allowed. The dose of the analgesic was determined by the investigator.

Subjects with hepatic impairment (in addition):
Start of any new medication (other than pain treatment) or any changes to a current dosage within 14 days prior to study drug administration was not allowed.

### 2.4.9 Treatment Compliance

Accountability and subject compliance were assessed by maintaining adequate study drug dispensing records. The Investigator was responsible for ensuring that dosing was administered in compliance with the protocol. Delegation of this task had to be clearly documented and approved by the Investigator. Compliance was further confirmed by bioanalytical assessment of E4 in plasma samples (see Section 2.5.5.1).

### 2.5 Pharmacokinetic, Safety, and Exploratory Measurements and Variables

The present study was performed to assess safety and PK parameters following a single dose of E4; this study did not comprise efficacy or pharmacodynamic assessments.

### 2.5.1 Schedules of Assessments

A Schedule of Assessments is presented in Table 2. PK sampling is presented in Table 3.

TABLE 2
Schedule of Assessments

| | Study Period | | | | | | | | | | | |
|---|---|---|---|---|---|---|---|---|---|---|---|---|
| | | | | | | | | | | | | Follow-up |
| | Screening | Assessment Period | | | | | | | | | | Visit¹ |
| | Study Day | | | | | | | | | | | |
| | Day −28 to | | Day | Day | Day | Day | Day | Day | Day | Day | Day | Day |
| | Day −2 | Day −1 | 1 | 2 | 3 | 4 | 5 | 6 | 7 | 8 | 9 | 14 ± 2 |
| Admission | | X | | | | | | | | | | |
| Discharge² | | | | | | X | | | | | (X) | |
| Informed Consent | X | | | | | | | | | | | |
| Inclusion/Exclusion | X | X | | | | | | | | | | |
| criteria | | | | | | | | | | | | |
| Medical history (incl. | X | | | | | | | | | | | |
| smoking/alcohol) | | | | | | | | | | | | |
| Demographics | X | | | | | | | | | | | |
| Physical examination³ | X | X | | | X | | | | | | | X |
| Gynecological | X | | | | | | | | | | | |
| examination⁴ | | | | | | | | | | | | |
| Height, Weight, BMI⁵ | X | X | | | | | | | | | | X |
| Vital Signs⁶ | X | X | X | X | X | X | X | X | X | X | X | X |
| Hepatic function | X⁷ | | | | | | | | | | | X |
| assessment | | | | | | | | | | | | |
| Safety lab tests⁷ | X | X | | X | X | | X | | | | X | X |
| Serology⁸ | X | | | | | | | | | | | |
| FSH⁹ | X | | | | | | | | | | | |
| Pregnancy test¹⁰ | X | X | | | | | | | | | | X |
| Drugs and alcohol abuse | X | X | | | | | | | | | | |
| screening¹¹ | | | | | | | | | | | | |
| Error! Reference | | | X | | | | | | | | | |
| source not found. | | | | | | | | | | | | |
| administration | | | | | | | | | | | | |
| PK blood sampling¹² | | | X | X | X | X | X | X | X | X | X | |
| AE/TEAE monitoring | X | X | X | X | X | X | X | X | X | X | X | X |
| Concomitant medication | | X | X | X | X | X | X | X | X | X | X | X |
| Prior medication | X | | | | | | | | | | | |

Note:
AE = adverse event;
BMI = body mass index;
FSH = follicle stimulating hormone;
h = hour;
PK = pharmacokinetic;
TEAE = treatment emergent AE
¹A follow-up visit was also to be performed in the case of early study termination of a subject.
²Discharge was to take place on Day 4 or, alternatively, on Day 9 after a prolonged in-house period, in agreement with Investigator and subject.
³Complete physical examination at screening and follow-up, Day −1 and Day 3: symptom orientated physical examination.
⁴Consisting of a breast examination, a transvaginal ultrasound, and a Pap smear (Pap smear only if none has been performed within 1 year prior to screening).
⁵Height at Screening only.
⁶Blood pressure, pulse rate, and respiratory rate measurements in supine position after a rest of at least 5 minutes; on Day 1: pre-dose, 2 h, 4 h, 6 h, 8 h, 10 h, 12 h post-dose; Day 2: 24 h, 36 h post-dose; Day 3: 48 h, 60 h post-dose, Day 4, Day 5, Day 6, Day 7, Day 8, Day 9 and Follow-up. Body temperature at Screening, Day −1, Day 1: pre-dose, 12 h post-dose; Day 3: 48 h post-dose; Day 4, Day 5, and Follow-up.
⁷Clinical chemistry, including coagulation, haematology and urinalysis (in fasted state).
⁸Hepatitis C virus antibodies, human immunodeficiency virus antibodies ½ and hepatitis B surface antigen.
⁹For the confirmation of the postmenopausal status if necessary.
¹⁰For non-hysterectomized women only. At screening and Follow-up test is done from serum, at Day −1 from urine.
¹¹Urine drug test and alcohol breath test.
¹²Blood PK samples will be collected according to Table 3.

TABLE 3
Pharmacokinetic Blood Sampling Schedule for Estetrol and its Metabolites
Sample collection time relative to dosing

| | |
|---|---|
| Study Day | Time point |

| 1 | Pre-dose (after overnight fast and within 60 min prior to dosing) | |
|---|---|---|
| | 15 min | (±1 min) |
| | 30 min | (±2 min) |
| | +1 h | (±5 min) |
| | +1.5 h | (±5 min) |
| | +2 h | (±5 min) |
| | +3 h | (±5 min) |
| | +4 h | (±10 min) |
| | +6 h | (±10 min) |
| | +8 h | (±10 min) |
| | +10 h | (±10 min) |
| | +12 h | (±15 min) |
| | +14 h | (±15 min) |
| | +16 h | (±30 min) |
| 2 | +24 h | (±30 min) |
| | +36 h | (±30 min) |
| 3 | +48 h | (±60 min) |
| 4 | +72 h | (±60 min) |
| 5 | +96 h | (±60 min) |
| 6 | +120 h | (±60 min) |
| 7 | +144 h | (±60 min) |
| 8 | +168 h | (±60 min) |
| 9 | +192 h | (±60 min) |

### 2.5.2 Pharmacokinetic Measurements

A Schedule of Assessments is presented in Table 2. PK sampling is presented in Table 3.

2.5.2.1 Blood Sampling Details on sample collection, handling, storage, and shipping were described in the laboratory manual prepared by the CRO.

### 2.5.3 Safety and Tolerability Measurements

Safety and tolerability assessments consisted of treatment-emergent AEs (TEAEs), clinical laboratory, vital signs, physical examination, and gynecological examination. Assessments were performed in accordance with Table 2 and Table 3 in Section 2.5.1.

### 2.5.3.1 Adverse Events

AEs/TEAEs were recorded after signing of the ICF until completion of the follow up visit. Any clinically significant observations in results of clinical laboratory, vital signs, or physical examinations were recorded as AEs.

A TEAE is defined as any event not present prior to administration of the study drug or any event already present that worsens in either severity or frequency following exposure to the study drug.

An adverse event (AE) which occurred after signing of ICF and prior to administration of the study drug was considered a pre-treatment AE.

At several time points before and after drug administration, subjects were asked non leading questions to determine the occurrence of AEs/TEAEs. Subjects were asked in general terms about any AEs/TEAEs at regular intervals during the study. In addition, all AEs/TEAEs reported spontaneously during the course of the study were recorded.

All answers were interpreted by the investigator using the Medical Dictionary for Regulatory Activities (MedDRA; Version 22.1) for AEs/TEAEs and were recorded in the AEs Record.

Pregnancy was to be monitored along with follow-up, if warranted.

### 2.5.3.2 Clinical Laboratory

The below listed tests were to be performed by the local laboratories at time points indicated in the Schedule of Assessments (Table 2).

TABLE 4
Clinical Laboratory Tests

| Hematology and | | | |
|---|---|---|---|
| Coagulation | Serum Chemistry | Urinalysis | Additional Tests |
| Hemoglobin | Sodium | pH | Serology: anti-HIV-1/2, |
| Hematocrit | Potassium | Specific gravity | HBsAg, anti-HCV^{b} |
| Erythrocytes | Chloride | Color | FSH test |
| Platelets | Calcium | Protein | for postmenopausal |
| Leukocytes | Inorganic phosphate | Glucose | women |
| Neutrophils^{a} | Urea | Ketones | Serum/urine pregnancy |
| Eosinophils^{a} | Creatinine^{c} | Hemoglobin | test for women of |
| Lymphocytes^{a} | Uric acid | (erythrocytes) | childbearing potential, |
| Monocytes^{a} | Total bilirubin | Leukocytes | including women |
| Basophils^{a} | Direct bilirubin | Microscopic | whose last |
| International Normalized | Alanine aminotransferase | analysis, if urine is | menstruation |
| Ratio (INR) | (ALT) | positive for protein, | was <1 |
| Partial thromboplastin | Aspartate aminotransferase | leukocytes or | year before |
| time (aPTT) | (AST) | hemoglobin | screening |
| Prothrombin time | Gamma glutamyl transferase | | Urine drugs of abuse test |
| | (GGT) | | including cannabinoids, |
| | Alkaline phosphatase (ALP) | | amphetamines, |
| | Lactate dehydrogenase | | methamphetamines, |
| | (LDH) | | opiates, methadone, |
| | Creatine kinase (CK) | | cocaine, |
| | Amylase | | benzodiazepines, |
| | Lipase | | tricyclic antidepressants, |
| | Triglycerides | | and barbiturates |
| | Total cholesterol | | Alcohol breath test |
| | Total protein | | |
| | Albumin | | |
| | Glucose | | |
| | Thyroid stimulating | | |
| | hormone (TSH) | | |
| | Free thyroxine (fT4) | | |

CLCr = creatinine clearance; FSH = follicle stimulating hormone; HBsAg = hepatitis B surface antigen; HCV = hepatitis C virus; HIV = human immunodeficiency virus; PCR = polymerase chain reaction.
^{a}Subpopulations of leukocytes to be determined in absolute measures and as percentage of total leukocytes
^{b}If positive, confirmation by PCR
^{c}For CLCr calculation using Cockcroft-Gault: CLCr = (((140 − age [years]) * weight [kg])/(72 * S-creatinine [μmol/l]/88.4))) * 0.85 (if female)

The clinical laboratory was to clearly mark all laboratory test values that were outside the normal range and the Investigator was to indicate the clinically significance according to the applicable CRO Standard Operating Procedure (SOP). The procedures for the collection, handling, and shipping of laboratory samples were specified in the laboratory manual(s) provided to the study site.

An estimated total volume of 285 mL blood per subject was collected throughout the study.

### 2.5.3.3 Vital Signs

Systolic and diastolic blood pressure and pulse were recorded after the subject had been resting for at least 5 minutes in the supine position at time points indicated in the Schedule of Assessments (Table 2). These assessments were made using an automated device. Body temperature and respiratory rate were measured subsequently.

### 2.5.3.4 Physical Examination

A complete physical examination consisting of a review of all body systems was performed at time points indicated in the Schedule of Assessments (Table 2). A symptom-directed physical examination could be conducted at any time per Investigator's discretion.

Height was recorded at the Screening visit only. Weight was measured, and BMI calculated at time points indicated in the Schedule of Assessments (Table 2).

A gynecological examination (consisting of a breast examination, a transvaginal ultrasound, and a Pap smear [Pap smear only if none had been performed within 1 year prior to screening]) was performed in accordance with the Schedule of Assessments in Table 2.

### 2.5.4 Appropriateness of Measurements

The assessments that were made in this study are standard, and generally recognized as reliable, accurate, and relevant. Valid PK assays are the standard measurement for determining changes in blood levels of a drug and its pharmacologic effects with varying hepatic function. Collection of AEs/TEAEs, safety laboratory tests, and physical examinations are standard methods for evaluating the safety of new medicines. Collectively, these are appropriate measures to address the study objectives.

All procedures are documented in the CRO SOPs.

### 2.5.4.1 Timing of Assessments

For PK, pre-dose samples were obtained after overnight fasting within 60 minutes before dosing. Post-dose samples were obtained with time margins as described in Table 3.

For safety assessments, pre-dose assessments were to be performed between waking up and dosing. For safety assessments up to 2.5 h post-dose a time-window of +15 minutes was allowed. Thereafter, serial post-dose assessments (eg, multiple assessments within any given day) were to be performed with time margins of +10% of the time that has passed since (last) dosing; incidental post-dose assessments (eg, 1 or 2 assessments within a given day) were to be performed within +3 h of the planned scheme time, provided that it remained on the same part of the day and was not shifted from pre-dose to post-dose or vice versa. In the event assessments were planned for the same scheme time, the following sequence should have been followed: (1) Physical examination; (2) vital signs assessments; (3) blood sampling, with PK blood sampling exactly on time and (4) meal (if applicable).

### 2.5.5 Pharmacokinetic and Safety Variables

### 2.5.5.1 Pharmacokinetic Variables

Calculation of non-compartmental PK parameters were done using WinNonlin™ Professional, Version 6.3 or higher.

### 2.5.5.1.1 Primary Pharmacokinetic Parameters

The primary PK parameters to be analyzed for E4 in plasma:
Cmax=maximum observed plasma concentration
AUCinf=area under the concentration-time curve (AUC) from time zero extrapolated to infinity

### 2.5.5.1.2 Secondary Pharmacokinetic Parameters

The secondary PK parameters to be analyzed for E4 and its metabolites in plasma:
Cmax=maximum observed plasma concentration (only for metabolites)
AUCinf=AUC from time zero extrapolated to infinity (only for metabolites)
AUC0-24 h=AUC from time 0 to 24 h post-dose
AUClast=AUC up to the last time with concentration above the lower limit of quantitation
Tmax=time to attain Cmax
T½=terminal elimination half-life
CL/F=apparent clearance (only for E4)
Λz=terminal elimination phase rate constant
Vz/F=apparent volume of distribution during terminal phase (only for E4)

Additional PK parameters may be calculated as deemed appropriate.

### 2.5.5.2 Safety Variables

The safety variables to be measured include but are not limited to the variables as given below:
Clinical laboratory
Vital signs
Physical examination
TEAEs

### 2.5.6 Drug Concentration Measurements

The analysis of E4 and the metabolites E4-3-glucuronide and E4-16-glucuronide in plasma samples were performed at the Bioanalytical Laboratory of the CRO using a validated liquid chromatography-mass spectrometry/mass spectrometry method.

### 2.6 Statistical Methods Planned in the Protocol and Determination of Sample Size

### 2.6.1 Analysis Sets

### 2.6.1.1 Safety Set

All subjects who have received a dose of E4 monohydrate.

### 2.6.1.2 Pharmacokinetic Set

All subjects who received at least 1 dose of E4 monohydrate and provided sufficient bioanalytical assessment results to calculate reliable estimates of the PK parameters.

### 2.6.2 Statistical and Analytical Plan for Pharmacokinetic, Safety, and Exploratory Evaluation

Statistical analysis was performed using the appropriate procedures in SAS® Version 9.4 or above.

All data were summarized using descriptive statistics and were listed and summarized in tabular and/or graphical form.

### 2.6.2.1 Pharmacokinetic Evaluation

Individual and mean plasma concentrations at each sampling time point for E4 are presented by listings and descriptive summary statistics including means, geometric means (GMs), medians, ranges, standard deviations (SD) and coefficients of variation (CV). Individual, mean and/or median concentrations per hepatic function group were plotted versus time on semilogarithmic scales.

PK parameters were calculated by non-compartmental analysis. PK parameters were analysed using descriptive statistics and are presented by hepatic function group.

Summary statistics of PK parameters including means, GMs, medians, ranges, SDs, and CVs are presented by hepatic function group.

Analysis of variance (ANOVA) were used to compare primary PK parameters of E4 (Cmax and AUCinf) between subjects with hepatic impairment and subjects with normal hepatic function in an exploratory approach, using the log-transformed value of each PK parameter as dependent variable, and the hepatic impairment group (normal, mild, moderate, and severe) as a fixed classification factor. Geometric least squares means were used to calculate the ratios of primary PK parameters for each hepatic impairment group to those in the control group, along with 90% confidence intervals (CIs).

Additionally, the impact of age and BMI on the effect of hepatic impairment were evaluated using an ANOVA model similar as the ANOVA model described above, but with the interaction between each factor and the hepatic impairment group included as an additional factor.

The relationship between log-transformed PK parameters (Cmax and AUCinf) and continuous parameters (albumin, total bilirubin, and prothrombin time) used for the Child-Pugh score was explored by a linear regression approach. An additional linear regression stratified by BMI or age groups could be performed in case of significant interaction with hepatic impairment groups identified using the ANOVA model. Further exploratory analyses of the relationship of hepatic impairment and PK could be performed if indicated.

Secondary PK parameters were analysed descriptively.

2.6.2.2 Evaluation of Safety and Tolerability Safety and tolerability were assessed through TEAEs, clinical laboratory, vital signs, and physical examination findings, and any other parameter that was relevant for safety assessment.

All individual safety results are listed and descriptive statistics including change from baseline were calculated, where applicable.

### 2.6.2.2.1 Adverse Events

A listing of all individual AEs is provided. Summary tables of TEAEs are presented by system organ class (SOC) based on the MedDRA terminology list (preferred terms): a table containing the number of subjects experiencing AEs by hepatic function, a table containing the number of subjects experiencing SAEs by hepatic function (if applicable), a table by hepatic function and relationship, and a table by hepatic function and severity.

All TEAE summaries are presented alphabetically by SOC, with preferred terms (PTs) sorted in decreasing order of frequency within each SOC based on the MedDRA.

A summary of the number and percentage of subjects reporting TEAEs, SAEs, TEAEs leading to study drug discontinuation, or TEAEs by severity, is presented by hepatic function group.

A summary of the number and percentage of subjects reporting each TEAE is presented by hepatic function group. Counting were done by subject only, not by event; subjects were only counted once within each body system or PT.

A summary of the number and percentage of subjects reporting each TEAE is presented by relationship to study drug (as recorded on the eCRF) and by hepatic function group. Subjects with multiple events within a particular SOC or PT are counted as related unless no events were related in that SOC/PT.

A summary of the number and percentage of subjects reporting each TEAE are presented by severity (as recorded on eCRF) and by hepatic function group. Subjects with multiple events within a particular SOC or PT are counted under the category of their most severe event within that SOC or PT.

All AEs (including non-treatment-emergent events) recorded on the eCRF are listed by subject.

A separate listing of AEs leading to study drug discontinuation is provided by subject.

### 2.6.2.2.2 Clinical Laboratory

All laboratory data were converted to Système International Units for reporting and processing purposes.

Clinical laboratory data are listed accompanied by an indication if the parameter is outside the reference range and a summary listing of all data assessed by the Investigator as clinically significant were prepared.

A descriptive statistics summary of continuous laboratory results for clinical chemistry including haematology, coagulation, and derived changes from baseline is provided by hepatic function group and scheduled time point.

All laboratory data are listed by subject.

A separate listing of clinically significant abnormal values is provided.

### 2.6.2.2.3 Physical Examination

Physical examination data are listed by subject.

### 2.6.3 Determination of Sample Size

No formal sample size calculation was performed as there was no prior information on PK variability of E4 in subjects with hepatic impairment. The number of subjects to be enrolled was chosen based on practical considerations in compliance with the applicable guidance for clinical studies with subjects with hepatic impairment.

### 2.7 Safety Laboratory Data Review

Safety laboratory results of the first 2 subjects of each group were reviewed to assess a potential impact of E4 on liver function (increase of liver enzymes) before enrolment of next subject(s). Results from Day 2, Day 3 and Day 5 were considered and compared to results from Day −1 (baseline).

A potential fluctuation of liver function was to be expected. If the results of liver enzymes exceeded the baseline value by 1.5-fold (either upper normal limit or actual value if higher than upper normal limit on Day −1) the case was further discussed within the CRO and with the Sponsor for follow up of the situation prior to further enrolment of subjects into that group.

### 2.8. Demographics

A total of 32 female subjects between 48 and 70 years of age and with a BMI between and 18.5 and 34.7 kg/m2 participated in the study. All subjects were white, none were of multiple race, or of Hispanic or Latino ethnicity (Table 7).

TABLE 5
Summary of Demographic Characteristics

| | Category or | Normal | Mild | Moderate | Severe | Total |
|---|---|---|---|---|---|---|
| | Statistics | (N = 8) | (N = 8) | (N = 8) | (N = 8) | (N = 32) |
| Gender (n) | Female | 8 | 8 | 8 | 8 | 32 |
| Race (n) | White | 8 | 8 | 8 | 8 | 32 |
| | American | 0 | 0 | 0 | 0 | 0 |
| | Indian or Alaska | | | | | |
| | Native | | | | | |
| | Asian | 0 | 0 | 0 | 0 | 0 |
| | Black or African | 0 | 0 | 0 | 0 | 0 |
| | American | | | | | |
| | Native | 0 | 0 | 0 | 0 | 0 |
| | Hawaiian or | | | | | |
| | other Pacific | | | | | |
| | Islander | | | | | |
| | Other | 0 | 0 | 0 | 0 | 0 |
| Ethnicity (n) | Not Hispanic or | 8 | 8 | 8 | 8 | 32 |
| | Latino | | | | | |
| | Hispanic or | 0 | 0 | 0 | 0 | 0 |
| | Latino | | | | | |
| Age (years) | n | 8 | 8 | 8 | 8 | 32 |
| | Mean | 54.9 | 55.5 | 57.3 | 56.4 | 56.0 |
| | SD | 5.89 | 7.35 | 6.65 | 4.21 | 5.90 |
| | Median | 55.0 | 53.5 | 57.0 | 56.0 | 55.5 |
| | Min, Max | 48, 66 | 48, 70 | 48, 66 | 51, 63 | 48, 70 |
| Screening | n | 8 | 8 | 8 | 8 | 32 |
| Weight (kg) | Mean | 68.03 | 66.32 | 63.18 | 66.29 | 65.95 |
| Screening | n | 8 | 8 | 8 | 8 | 32 |
| Height (cm) | Mean | 163.88 | 162.50 | 158.13 | 161.00 | 161.38 |
| Screening | n | 8 | 8 | 8 | 8 | 32 |
| BMI (kg/m²) | Mean | 25.50 | 25.03 | 25.30 | 25.46 | 25.32 |

BMI = body mass index;
Max = maximum;
Min = minimum;
N = number of subjects;
n = number of subjects as a unit;
SD = standard deviation

The medical history of the group of subjects with normal hepatic function was appropriate to the age of this population. Subjects with hepatic impairment showed disease characteristics that were in line with the origin of their underlying liver dysfunction or were secondary to this condition. Of the 32 female subjects, 3 were of childbearing potential.

### 3. Pharmacokinetic Results

### 3.1 Pharmacokinetic Results

### 3.1.1 E4 Plasma Concentrations

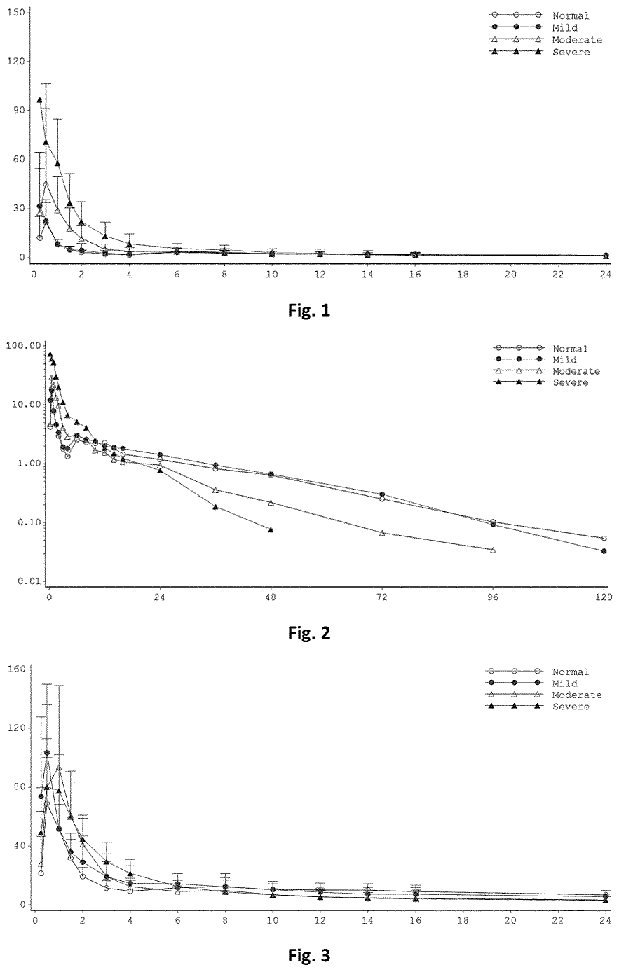

E4 was rapidly absorbed and reached its primary peak plasma concentration within 30 min of administration on average, within 15 min for the group of severe hepatic impairment, and no longer than 2 h for any of the subjects. E4 plasma concentration then rapidly declined before describing a smaller secondary peak in all groups except the group of subjects with severe hepatic impairment. After ~12 h, E4 entered a logarithmic-linear terminal elimination phase (FIG. 1 and FIG. 2).

The groups with moderate and severe hepatic impairment showed a faster decline compared to the groups with mild hepatic impairment and normal hepatic functioning. The concentrations of E4 remained above LLOQ in at least half of the subjects until 48 h post dose for the group with severe hepatic impairment, until 96 h for the group with moderate hepatic impairment, and for the group with mild hepatic impairment and the subjects with normal hepatic function this was until 144 h post dose, respectively (FIG. 2).

### 3.1.2 E4-3-Glucuronide Plasma Concentrations

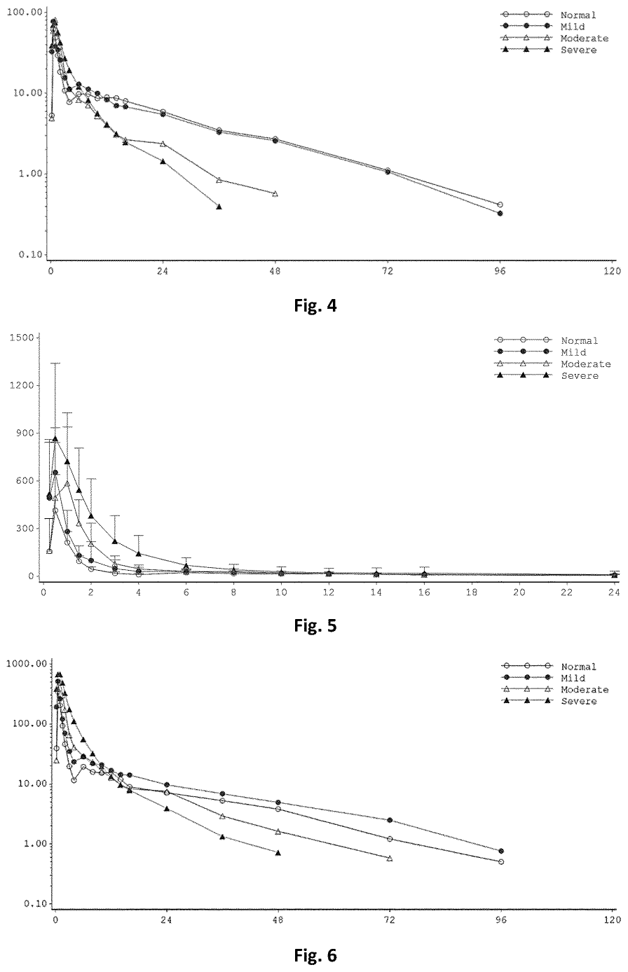

The arithmetic mean plasma concentrations of E4-3-glucuronide versus time are shown in FIG. 3 and GM plasma concentrations in FIG. 4. GM plasma concentrations reached a primary peak of a similar value within ~1 h in all groups, with all but the severe hepatic impairment group displaying a second peak.

The group with severe hepatic impairment showed the fastest decline compared to the groups with moderate hepatic impairment, mild hepatic impairment, and normal hepatic functioning. The concentrations of E4-3 remained above LLOQ in at least half of the subjects until 36 h post dose for the group with severe hepatic impairment, until 48 h for the group with moderate hepatic impairment, and until 96 h for the group with mild hepatic impairment and the subjects with normal hepatic function. 3.1.3 E4-16-glucuronide Plasma Concentrations

The arithmetic mean and GM plasma concentrations of E4-16-glucuronide versus time are shown in FIG. 5 and FIG. 6, respectively. The GM plasma concentrations reached a primary peak within ~1 h in all groups. The peak value was similar across the 3 groups of subjects with hepatic impairment, and higher than the peak value for the group of subjects with normal hepatic function. A secondary peak was reached before entering elimination phase in the groups with normal hepatic function and mild hepatic impairment.

The group with severe hepatic impairment showed the fastest decline compared to the groups with moderate hepatic impairment, mild hepatic impairment, and normal hepatic functioning. The concentrations of E4-16 remained above LLOQ in at least half of the subjects until 48 h post-dose for the group with severe hepatic impairment, until 72 h for the group with moderate hepatic impairment, and until 96 h for the group with mild hepatic impairment and the subjects with normal hepatic function.

### 3.1.4 Pharmacokinetic Parameters of E4 in Plasma

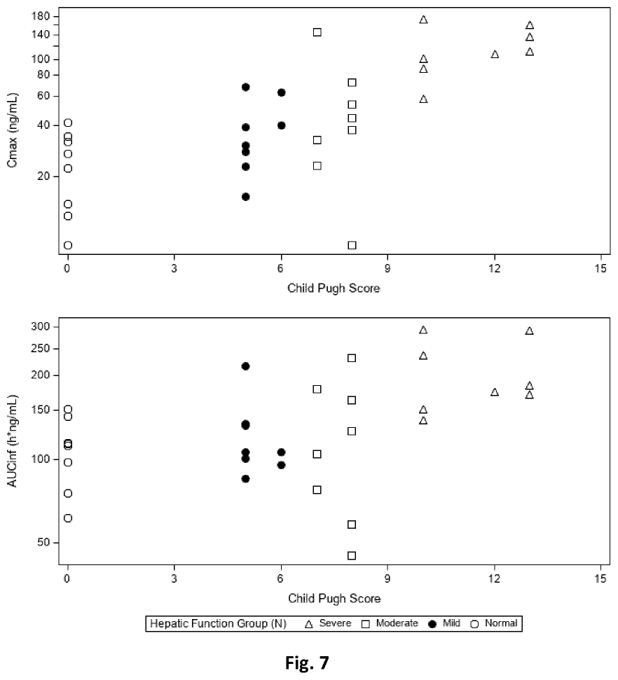

The effect of hepatic impairment on individual Cmax and AUCinf values is displayed in relation to the Child-Pugh Score in FIG. 7. All groups displayed a large inter-individual variability-Cmax and AUCinf values in the moderate hepatic impairment group spread over the largest range. In all subjects with severe hepatic impairment, Cmax was above the maximum value measured in the control group. In the mild and moderate hepatic impairment groups, the maximum Cmax was higher than in the control group. Minimum AUCinf measured in the severe hepatic impairment group was similar to the maximum AUCinf measured in the control group. In the mild and moderate hepatic impairment groups, the maximum AUCinf was higher than in the control group.

Summary statistics of primary and secondary PK parameters of E4 are presented in Table 8.

Compared to normal hepatic function, the GM values of Cmax of the parent compound E4 increased in all groups with hepatic impairment. The effect was most pronounced in the group with severe hepatic impairment, less in mild or moderate impairment, with the latter being inconclusive due to the high variability.

Mean, median, and GM AUCinf values were elevated in the severe impairment group (GM AUCinf ~1.9-fold), but comparable in the groups of mild impairment and normal hepatic function, and although apparently also for the group with moderate hepatic impairment, but given the high variability in this group the results are inconclusive (Table 12). Median Tmax ranged from 0.25 h to 0.50 h and did not suggest any relationship to the degree of hepatic impairment. The GM values of T½ were similar in subjects with normal hepatic function, mild impairment, and moderate impairment with high variability. Only in subjects with severe hepatic impairment a considerably lower GM T½ was suggested compared to the other groups. The last measurable concentration of E4 (Clast) was attained 192 h post-dose (the last measured timepoint) in subjects with normal hepatic function or with mild hepatic impairment, 144 h in subjects with moderate hepatic impairment, and 168 h in subjects with severe hepatic impairment. The apparent clearance CL/F was similar in groups with normal hepatic function and mild and moderate impairment, and presented a slight reduction in the group with severe impairment.

In subjects with normal hepatic function GM of Vz/F was 5996.6 L and decreased to 1201.4 L in subjects with severe hepatic impairment.

The variability of the primary PK parameters of E4 was moderate to high, (FIG. 7).

TABLE 6
Pharmacokinetic Parameters of E4 (Pharmacokinetic Set)

| PK Parameter | Summary | Normal | Mild | Moderate | Severe |
|---|---|---|---|---|---|
| (Unit) | Statistics | (N = 8) | (N = 8) | (N = 8) | (N = 8) |
| Cₘₐₓ | n | 8 | 8 | 8 | 8 |
| (ng/mL) | Mean | 23.878 | 38.363 | 52.410 | 117.025 |
| | SD | 12.1680 | 18.6620 | 42.5795 | 38.2407 |
| | % CV | 51.0 | 48.6 | 81.2 | 32.7 |
| | Median | 24.800 | 34.900 | 41.100 | 109.000 |
| | Min, Max | 7.72, 41.90 | 15.00, 68.00 | 7.78, 146.00 | 58.20, 174.00 |
| | GM | 20.750 | 34.460 | 39.350 | 111.220 |
| | Geo % CV | 65.8 | 53.8 | 104.4 | 36.2 |
| AUC_{inf} | n | 8 | 8 | 8 | 8 |
| (h*ng/mL) | Mean | 108.35 | 121.79 | 123.09 | 204.95 |
| | SD | 30.393 | 42.016 | 64.632 | 60.840 |
| | % CV | 28.1 | 34.5 | 52.5 | 29.7 |
| | Median | 112.62 | 105.96 | 115.30 | 179.68 |
| | Min, Max | 61.1, 151.0 | 84.8, 217.0 | 44.8, 231.6 | 138.0, 292.8 |
| | GM | 104.29 | 116.80 | 107.64 | 197.53 |
| | Geo % CV | 31.1 | 30.0 | 62.3 | 29.3 |
| AUC_{0-24 h} | n | 8 | 8 | 8 | 8 |
| (h*ng/mL) | Mean | 63.51 | 72.92 | 103.05 | 183.59 |
| | SD | 21.074 | 25.165 | 58.392 | 46.927 |
| | % CV | 33.2 | 34.5 | 56.7 | 25.6 |
| | Median | 63.80 | 66.29 | 92.61 | 171.67 |
| | Min, Max | 34.0, 102.3 | 51.2, 127.1 | 39.6, 197.8 | 133.9, 285.1 |
| | GM | 60.41 | 69.76 | 88.75 | 179.05 |
| | Geo % CV | 35.4 | 31.4 | 65.1 | 23.6 |
| AUCₗₐₛₜ | n | 8 | 8 | 8 | 8 |
| (h*ng/mL) | Mean | 107.02 | 120.69 | 122.12 | 203.91 |
| | SD | 30.527 | 42.245 | 64.680 | 60.820 |
| | % CV | 28.5 | 35.0 | 53.0 | 29.8 |
| | Median | 111.36 | 105.22 | 113.73 | 178.03 |
| | Min, Max | 59.2, 150.1 | 82.4, 216.4 | 44.2, 230.9 | 137.3, 292.0 |
| | GM | 102.85 | 115.60 | 106.58 | 196.47 |
| | Geo % CV | 31.9 | 30.5 | 62.8 | 29.4 |
| Tₘₐₓ (h) | n | 8 | 8 | 8 | 8 |
| | Median | 0.475 | 0.375 | 0.500 | 0.250 |
| | Min, Max | 0.23, 1.00 | 0.23, 1.92 | 0.25, 1.48 | 0.25, 1.00 |
| T_{1/2} (h) | n | 8 | 8 | 8 | 8 |
| | Mean | 22.308 | 22.063 | 16.573 | 8.971 |
| | SD | 5.6626 | 11.0157 | 8.8768 | 4.3007 |
| | % CV | 25.4 | 49.9 | 53.6 | 47.9 |
| | Median | 22.717 | 16.220 | 14.272 | 6.260 |
| | Min, Max | 15.28, 29.69 | 13.57, 43.03 | 7.71, 34.46 | 5.86, 17.36 |
| | GM | 21.670 | 20.070 | 14.790 | 8.220 |
| | Geo % CV | 26.3 | 47.1 | 53.6 | 44.9 |
| CL/F (L/h) | n | 8 | 8 | 8 | 8 |
| | Mean | 200.09 | 177.13 | 214.77 | 104.83 |
| | SD | 65.631 | 44.624 | 126.005 | 28.453 |
| | % CV | 32.8 | 25.2 | 58.7 | 27.1 |
| | Median | 177.60 | 188.75 | 175.13 | 111.40 |
| | Min, Max | 132.5, 327.1 | 92.2, 235.9 | 86.4, 446.3 | 68.3, 145.0 |
| | GM | 191.77 | 171.23 | 185.80 | 101.25 |
| | Geo % CV | 31.1 | 30.0 | 62.3 | 29.3 |
| λz (1/h) | n | 8 | 8 | 8 | 8 |
| | Mean | 0.0329 | 0.0373 | 0.0520 | 0.0905 |
| | SD | 0.00847 | 0.01404 | 0.02395 | 0.03248 |
| | % CV | 25.7 | 37.6 | 46.1 | 35.9 |
| | Median | 0.0307 | 0.0428 | 0.0493 | 0.1108 |
| | Min, Max | 0.023, 0.045 | 0.016, 0.051 | 0.020, 0.090 | 0.040, 0.118 |
| | GM | 0.0300 | 0.0300 | 0.0500 | 0.0800 |
| | Geo % CV | 26.3 | 47.1 | 53.6 | 44.9 |
| Vz/F (L) | n | 8 | 8 | 8 | 8 |
| | Mean | 6594.8 | 5829.2 | 4756.1 | 1263.4 |
| | SD | 2961.56 | 3898.49 | 2985.72 | 399.17 |
| | % CV | 44.9 | 66.9 | 62.8 | 31.6 |
| | Median | 6428.8 | 4800.6 | 4152.0 | 1220.1 |
| | Min, Max | 2921, 11539 | 1805, 14645 | 1564, 9558 | 599, 1814 |
| | GM | 5996.6 | 4958.2 | 3963.2 | 1201.4 |
| | Geo % CV | 50.6 | 65.6 | 73.6 | 36.5 |

AUC = area under the concentration-time curve; AUC_{inf} = AUC from time zero extrapolated to infinity; AUCₗₐₛₜ = AUC up to the last time with concentration above the lower limit of quantitation; AUC_{0-24 h} = AUC from time 0 to 24 h post-dose; CL/F = apparent clearance; Cₘₐₓ = maximum observed plasma concentration; Geo = geometric; GM = geometric mean; λz = terminal elimination phase rate constant; Max = maximum; Min = minimum; N = number of subjects of the PK set; n = number of subjects analyzed; PK = pharmacokinetic; SD = standard deviation; Tₘₐₓ = time to attain Cₘₐₓ; T_{1/2} = terminal elimination half-life; Vz/F = apparent volume of distribution during terminal phase; % CV = percent coefficient of variation.

### 3.1.5 Pharmacokinetic Parameters of E4-3-Glucuronide in Plasma

Summary statistics for the metabolite E4-3-glucuronide are presented in Table 9.

E4-3-glucuronide GMs of Cmax and AUC0-24 were comparable across all hepatic function groups. GMs of AUCinf showed a slight decrease in moderate and severe hepatic impairment groups when compared to the normal hepatic function group.

Median Cmax increased in the groups with hepatic impairment compared to the normal hepatic function group. Median AUCinf slightly decreased in the groups with moderate and severe hepatic impairment compared to the normal hepatic function group. Median AUCinf was the lowest in the group with severe hepatic impairment. Median AUC0-24 h was comparable with the normal hepatic functioning group for the mild and moderate hepatic impairment groups, and was slightly decreased in the group with severe hepatic impairment. Median AUClast decreased in the groups with hepatic impairment compared to the normal hepatic function group, the decrease was the largest in the group with severe hepatic impairment.

Maximum values for Cmax, AUCinf, AUC0-24 h, and AUClast were comparable between the group with severe hepatic impairment and the control group.

Tmax was reached within ~1 h in the group with severe hepatic impairment, within ~2 h in the mild and moderate hepatic impairment groups and due to an outlier after ~6 h in the group with normal hepatic function. However, the median was ~0.5 h in all groups except for the moderate hepatic impairment group where it was 1 h.

GM T½ was reduced in the groups of moderate and severe hepatic impairment, when compared to the normal hepatic function group and increased in the group with mild hepatic impairment. Mean and GM T½ were the lowest in the group with severe hepatic impairment.

TABLE 7
Pharmacokinetic Parameters of E4-3-glucuronide (Pharmacokinetic Set)

| PK Parameter | Summary | Normal | Mild | Moderate | Severe |
|---|---|---|---|---|---|
| (Unit) | Statistics | (N = 8) | (N = 8) | (N = 8) | (N = 8) |
| Cₘₐₓ | n | 8 | 8 | 8 | 8 |
| (ng/mL) | Mean | 73.238 | 118.113 | 122.863 | 96.250 |
| | SD | 27.4139 | 33.9047 | 49.9016 | 22.8747 |
| | % CV | 37.4 | 28.7 | 40.6 | 23.8 |
| | Median | 70.100 | 114.000 | 133.000 | 91.550 |
| | Min, Max | 27.40, 127.00 | 66.50, 168.00 | 41.40, 200.00 | 70.20, 130.00 |
| | GM | 68.240 | 113.610 | 111.990 | 93.970 |
| | Geo % CV | 44.8 | 31.2 | 52.9 | 23.6 |
| AUC_{inf} | n | 8 | 7 | 6 | 8 |
| (h*ng/mL) | Mean | 485.88 | 506.96 | 388.13 | 367.42 |
| | SD | 169.100 | 108.644 | 135.640 | 207.439 |
| | % CV | 34.8 | 21.4 | 34.9 | 56.5 |
| | Median | 495.16 | 483.73 | 412.85 | 305.49 |
| | Min, Max | 251.1, 725.5 | 398.7, 654.9 | 167.9, 515.7 | 218.5, 855.8 |
| | GM | 458.11 | 497.24 | 363.25 | 332.77 |
| | Geo % CV | 39.1 | 21.4 | 44.8 | 46.1 |
| AUC_{0-24 h} | n | 8 | 8 | 8 | 8 |
| (h*ng/mL) | Mean | 290.53 | 322.40 | 282.87 | 314.97 |
| | SD | 79.803 | 72.219 | 90.879 | 118.250 |
| | % CV | 27.5 | 22.4 | 32.1 | 37.5 |
| | Median | 309.09 | 302.66 | 299.08 | 264.88 |
| | Min, Max | 155.0, 419.3 | 244.2, 460.9 | 157.0, 411.4 | 211.5, 568.7 |
| | GM | 279.86 | 315.86 | 269.04 | 299.07 |
| | Geo % CV | 31.1 | 21.6 | 35.8 | 34.0 |
| AUCₗₐₛₜ | n | 8 | 8 | 8 | 8 |
| (h*ng/mL) | Mean | 474.51 | 483.07 | 336.84 | 360.81 |
| | SD | 168.243 | 106.771 | 135.590 | 207.068 |
| | % CV | 35.5 | 22.1 | 40.3 | 57.4 |
| | Median | 485.66 | 440.22 | 325.82 | 296.24 |
| | Min, Max | 243.2, 708.2 | 378.4, 644.4 | 164.8, 505.7 | 211.2, 847.9 |
| | GM | 446.22 | 473.28 | 311.58 | 325.76 |
| p | Geo % CV | 40.1 | 21.6 | 45.3 | 46.9 |
| Tₘₐₓ (h) | n | 8 | 8 | 8 | 8 |
| | Median | 0.500 | 0.490 | 1.000 | 0.510 |
| | Min, Max | 0.47, 5.98 | 0.23, 1.92 | 0.50, 1.97 | 0.25, 1.00 |
| T_{1/2} (h) | n | 8 | 8 | 6 | 8 |
| | Mean | 20.112 | 24.540 | 17.084 | 8.652 |
| | SD | 5.4240 | 8.9547 | 11.5614 | 4.3940 |
| | % CV | 27.0 | 36.5 | 67.7 | 50.8 |
| | Median | 18.583 | 24.692 | 14.153 | 6.277 |
| | Min, Max | 14.83, 29.75 | 13.43, 40.13 | 7.06, 38.16 | 3.68, 13.99 |
| | GM | 19.540 | 23.130 | 14.390 | 7.710 |
| | Geo % CV | 25.4 | 38.4 | 69.9 | 55.2 |
| λz (1/h) | n | 8 | 8 | 6 | 8 |
| | Mean | 0.0364 | 0.0318 | 0.0560 | 0.1005 |
| | SD | 0.00835 | 0.01178 | 0.03111 | 0.04889 |
| | % CV | 22.9 | 37.0 | 55.6 | 48.6 |
| | Median | 0.0373 | 0.0281 | 0.0502 | 0.1104 |
| | Min, Max | 0.023, 0.047 | 0.017, 0.052 | 0.018, 0.098 | 0.050, 0.189 |
| | GM | 0.0400 | 0.0300 | 0.0500 | 0.0900 |
| | Geo % CV | 25.4 | 38.4 | 69.9 | 55.2 |

AUC = area under the concentration-time curve;
AUC_{inf} = AUC from time zero extrapolated to infinity;
AUCₗₐₛₜ = AUC up to the last time with concentration above the lower limit of quantitation;
AUC_{0-24 h} = AUC from time 0 to 24 h post-dose;
Cₘₐₓ = maximum observed plasma concentration;
Geo = geometric;
GM = geometric mean;
λz = terminal elimination phase rate constant;
Max = maximum;
Min = minimum;
N = number of subjects of the PK set;
n = number of subjects analyzed;
PK = pharmacokinetic;
SD = standard deviation;
Tₘₐₓ = time to attain Cₘₐₓ;
T_{1/2} = terminal elimination half-life;
Vz/F = apparent volume of distribution during terminal phase;
% CV = percent coefficient of variation

### 3.1.6 Pharmacokinetic Parameters of E4-16-Glucuronide in Plasma

Summary statistics for metabolite E4-16-glucuronide are presented in Table 10.

E4-16-glucuronide medians, means and GMs of Cmax, AUCinf, and AUC0-24 h increased in the hepatic impairment groups, most pronounced in the group with severe impairment. It should be noted that % CV was high for Cmax in normal subjects and for AUCs in the severe hepatic impairment group.

Maximum values for Cmax, AUCinf, AUC0-24 h, and AUClast increase multiple folds in the group with severe hepatic impairment compared with the control group (Cmax: ~2-fold, AUCinf: ~4-fold; AUC0-24 h: ~5 fold, and AUClast: ~4-fold).

The median Tmax was ~0.5 h for all groups except for the moderate hepatic impairment group where it was ~1.0 h.

GM T½ was reduced in the groups of moderate and severe hepatic impairment, when compared to the normal hepatic function group and increased in the group with mild hepatic impairment.

TABLE 8
Pharmacokinetic Parameters of E4-16-glucuronide (Pharmacokinetic Set)

| PK Parameter | Summary | Normal | Mild | Moderate | Severe |
|---|---|---|---|---|---|
| (Unit) | Statistics | (N = 8) | (N = 8) | (N = 8) | (N = 8) |
| Cₘₐₓ | n | 8 | 8 | 8 | 8 |
| (ng/mL) | Mean | 419.750 | 745.750 | 785.625 | 952.625 |
| | SD | 219.7146 | 233.9809 | 297.5111 | 354.5045 |
| | % CV | 52.3 | 31.4 | 37.9 | 37.2 |
| | Median | 513.000 | 712.500 | 759.500 | 894.000 |
| | Min, Max | 105.00, 699.00 | 385.00, 1030.00 | 431.00, 1130.00 | 583.00, 1660.00 |
| | GM | 353.110 | 711.100 | 734.210 | 901.010 |
| | Geo % CV | 78.1 | 35.0 | 41.7 | 36.2 |
| AUC_{inf} | n | 8 | 7 | 7 | 8 |
| (h*ng/mL) | Mean | 946.46 | 1389.24 | 1518.70 | 2467.86 |
| | SD | 334.842 | 177.588 | 412.956 | 1779.722 |
| | % CV | 35.4 | 12.8 | 27.2 | 72.1 |
| | Median | 932.27 | 1358.39 | 1534.85 | 1959.93 |
| | Min, Max | 474.8, 1599.7 | 1192.1, 1735.8 | 761.8, 2046.8 | 1046.7, 6482.2 |
| | GM | 896.29 | 1380.13 | 1459.97 | 2081.97 |
| | Geo % CV | 36.8 | 12.2 | 33.1 | 63.8 |
| AUC_{0-24 h} | n | 8 | 8 | 8 | 8 |
| (h*ng/mL) | Mean | 676.36 | 1038.91 | 1293.40 | 2295.22 |
| | SD | 225.786 | 231.493 | 360.886 | 1500.633 |
| | % CV | 33.4 | 22.3 | 27.9 | 65.4 |
| | Median | 613.15 | 1061.93 | 1323.09 | 1922.86 |
| | Min, Max | 349.9, 1012.3 | 706.8, 1489.4 | 699.3, 1744.5 | 1046.0, 5542.7 |
| | GM | 643.44 | 1017.08 | 1244.09 | 1977.74 |
| | Geo % CV | 35.3 | 22.3 | 31.7 | 60.0 |
| AUCₗₐₛₜ | n | 8 | 8 | 8 | 8 |
| (h*ng/mL) | Mean | 919.86 | 1364.27 | 1435.97 | 2443.30 |
| | SD | 333.145 | 211.899 | 416.680 | 1778.821 |
| | % CV | 36.2 | 15.5 | 29.0 | 72.8 |
| | Median | 894.59 | 1329.47 | 1459.15 | 1951.71 |
| | Min, Max | 440.8, 1560.6 | 1079.4, 1699.0 | 743.4, 2010.3 | 1041.8, 6457.4 |
| | GM | 867.85 | 1350.15 | 1375.56 | 2057.13 |
| | Geo % CV | 38.4 | 15.5 | 33.6 | 64.1 |
| Tₘₐₓ (h) | n | 8 | 8 | 8 | 8 |
| | Median | 0.500 | 0.490 | 0.985 | 0.500 |
| | Min, Max | 0.47, 1.00 | 0.23, 1.92 | 0.50, 1.48 | 0.25, 1.00 |
| T_{1/2} (h) | n | 8 | 7 | 7 | 8 |
| | Mean | 19.245 | 24.634 | 14.217 | 10.103 |
| | SD | 6.6536 | 5.5056 | 6.2696 | 6.2115 |
| | % CV | 34.6 | 22.3 | 44.1 | 61.5 |
| | Median | 17.755 | 25.699 | 12.381 | 8.542 |
| | Min, Max | 10.53, 27.88 | 17.35, 32.24 | 6.93, 24.25 | 2.82, 21.35 |
| | GM | 18.220 | 24.080 | 13.050 | 8.450 |
| | Geo % CV | 37.0 | 23.6 | 47.7 | 74.1 |
| λz (1/h) | n | 8 | 7 | 7 | 8 |
| | Mean | 0.0402 | 0.0295 | 0.0580 | 0.0995 |
| | SD | 0.01448 | 0.00702 | 0.02592 | 0.06898 |
| | % CV | 36.0 | 23.8 | 44.7 | 69.3 |
| | Median | 0.0391 | 0.0270 | 0.0560 | 0.0882 |
| | Min, Max | 0.025, 0.066 | 0.021, 0.040 | 0.029, 0.100 | 0.032, 0.246 |
| | GM | 0.0400 | 0.0300 | 0.0500 | 0.0800 |
| | Geo % CV | 37.0 | 23.6 | 47.7 | 74.1 |

AUC = area under the concentration-time curve;
AUC_{inf} = AUC from time zero extrapolated to infinity;
AUCₗₐₛₜ = AUC up to the last time with concentration above the lower limit of quantitation;
AUC_{0-24 h} = AUC from time 0 to 24 h post-dose;
Cₘₐₓ = maximum observed plasma concentration;
Geo = geometric;
GM = geometric mean;
λz = terminal elimination phase rate constant;
Max = maximum;
Min = minimum;
N = number of subjects of the PK set;
n = number of subjects analyzed;
PK = pharmacokinetic;
SD = standard deviation;
Tₘₐₓ = time to attain Cₘₐₓ;
T_{1/2} = terminal elimination half-life;
Vz/F = apparent volume of distribution during terminal phase;
% CV = percent coefficient of variation

### 3.1.7 Pharmacokinetic Statistical Analyses

The linear regression of Cmax and AUCinf of E4 against laboratory parameters used for grading of the severity of hepatic dysfunction in the Child-Pugh classification showed an inverse relationship to albumin and a direct relationship to bilirubin and prothrombin time (Table 11). The 90% CIs suggest a relevant relationship in all regressions (0 is excluded).

TABLE 9
Linear Regression of Primary PK Parameters of E4 and Laboratory
Parameters Used in Child-Pugh Score (Pharmacokinetic Set)

| | | | | 90% |
|---|---|---|---|---|
| | Laboratory | Number of | Slope | Confidence |
| PK Parameter | variable | Subjects | Estimate | Interval |
| Cₘₐₓ | Albumin (G/L) | 32 | −0.092 | (−0.136, −0.048) |
| (ng/mL) | Bilirubin In Blood | 31 | 0.018 | (0.01, 0.025) |
| | (Umol/L) | | | |
| | Prothrombin Time | 32 | 0.176 | (0.119, 0.233) |
| | (Sec) | | | |
| AUC_{inf} | Albumin (G/L) | 32 | −0.037 | (−0.062, −0.012) |
| (h*ng/mL) | | | | |
| | Bilirubin In Blood | 31 | 0.008 | (0.004, 0.013) |
| | (Umol/L) | | | |
| | Prothrombin Time | 32 | 0.078 | (0.044, 0.112) |
| | (Sec) | | | |

AUC_{inf} = Area under the concentration-time curve from time zero extrapolated to infinity;
Cₘₐₓ = maximum observed plasma concentration
Note:
The dependent variable is log-transformed primary PK parameter. The estimates and confidence intervals refer to the slope for the continuous laboratory parameters used in the Child-Pugh score in the linear regression.

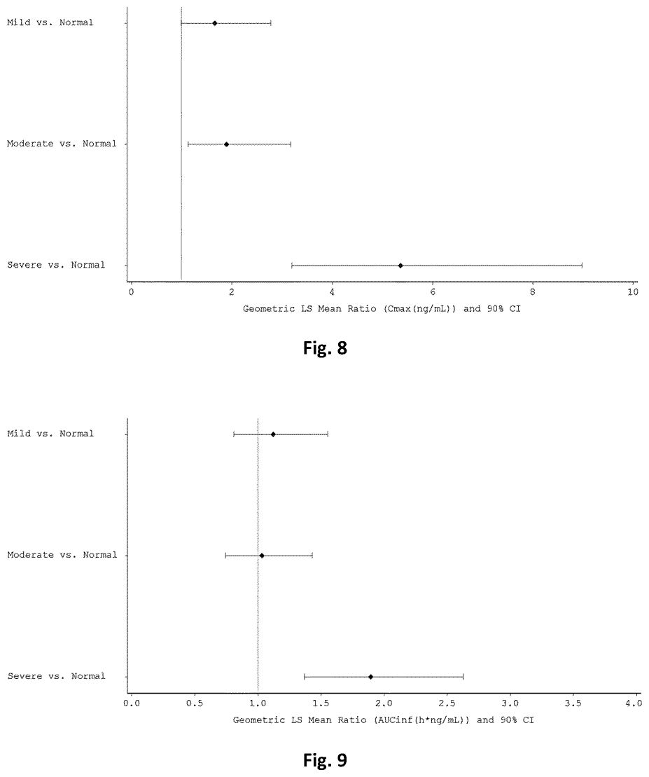

Using a one-way ANOVA, Cmax of E4 was ~5.4-fold higher in subjects with severe hepatic impairment (90% CI: 3.198-8.981) and ~1.9-fold higher in subjects with moderate hepatic impairment (90% CI: 1.132-3.178) when compared to the control group. Cmax for the group with mild hepatic impairment did not present a relevant difference from the control group [~1.7 fold (90% CI: 0.992-2.783)] (Table 12, FIG. 8).

In the group with severe hepatic impairment, AUCinf was ~1.9-fold higher (90% CI: 1.367-2.625) than in the control group (Table 12, FIG. 9). There is no relevant difference of AUCinf for the mild and moderate hepatic impairment groups compared to the control group [~1.1-fold (90% CI: 0.808-1.552) and ~1.0-fold (90% CI: 0.745-1.431), respectively].

TABLE 10
One-Way ANOVA of Primary Pharmacokinetic Parameters of
E4 among Hepatic Function Groups (Pharmacokinetic Set)

| | | | | | | Geometric Mean | |
|---|---|---|---|---|---|---|---|
| Comparison | | Test | | Reference | | Ratio (Test/ | |
| (Test vs. | | | Geometric | | Geometric | Reference) | |
| Reference) | PK Parameter | n | LS Mean | n | LS Mean | Estimate | 90% CI |
| Mild vs. | Cₘₐₓ (ng/mL) | 8 | 34.5 | 8 | 20.8 | 1.661 | (0.991, |
| Normal | | | | | | | 2.783) |
| | AUC_{inf} (h*ng/mL) | 8 | 117 | 8 | 104 | 1.120 | (0.808, |
| | | | | | | | 1.552) |
| Moderate vs. | Cₘₐₓ (ng/mL) | 8 | 39.4 | 8 | 20.8 | 1.896 | (1.132, |
| Normal | | | | | | | 3.178) |
| | AUC_{inf} (h*ng/mL) | 8 | 108 | 8 | 104 | 1.032 | (0.745, |
| | | | | | | | 1.431) |
| Severe vs. | Cₘₐₓ (ng/mL) | 8 | 111 | 8 | 20.8 | 5.359 | (3.198, |
| Normal | | | | | | | 8.981) |
| | AUC_{inf} (h*ng/mL) | 8 | 198 | 8 | 104 | 1.894 | (1.367, |
| | | | | | | | 2.625) |

ANOVA = analysis of variance; AUC_{inf} = Area under the concentration-time curve from time zero extrapolated to infinity; CI = confidence interval; Cₘₐₓ = maximum observed plasma concentration; LS = least squares; PK = pharmacokinetic.

In the 2-way ANOVA, the 4 hepatic function groups (mild, moderate, severe, and normal) and 2 BMI groups (18.5 kg/m2 to <25 kg/m2, and >=25 kg/m2) were compared against the primary PK parameters (Cmax and AUCinf) (Table 13). The results suggested a relevant impact of the hepatic function on the PK parameters while there was no effect of BMI on these PK parameters. In addition, there was no interaction between the hepatic function groups and BMI groups, thus suggesting that hepatic function was not related to BMI. The study was exploratory and not powered, thus P-values have to be interpreted with caution.

TABLE 11
Two-Way ANOVA of Primary Pharmacokinetic Parameters among
Hepatic Function and BMI Groups of E4 (Pharmacokinetic Set)

| Dependent | Independent | Sum of | | Mean | | |
|---|---|---|---|---|---|---|
| Variable | Variable | Squares | DF | Square | F | p-value |
| Cₘₐₓ | Hepatic | 11.9 | 3 | 3.97 | 10.9 | 0.0001 |
| (ng/mL) | BMI | 0.020 | 1 | 0.020 | 0.054 | 0.8175 |
| | Hepatic*BMI | 1.58 | 3 | 0.526 | 1.45 | 0.2537 |
| AUC_{inf} | Hepatic | 2.36 | 3 | 0.788 | 5.21 | 0.0072 |
| (h*ng/mL) | BMI | 0.355 | 1 | 0.355 | 2.34 | 0.1401 |
| | Hepatic*BMI | 0.147 | 3 | 0.049 | 0.324 | 0.8082 |

ANOVA = analysis of variance; AUC_{inf} = Area under the concentration-time curve from time zero extrapolated to infinity; BMI = body mass index; Cₘₐₓ = maximum observed plasma concentration; DF = degree of freedom.
Note:
Hepatic and BMI here refers respectively to the hepatic function group and BMI group.

### 3.2 Pharmacokinetic Evaluation

### 3.2.1 E4 Pharmacokinetics

GM Cmax was ~1.7-fold higher in the group with mild hepatic impairment, ~1.9-fold higher in the group with moderate hepatic impairment, and ~5.4-fold higher in the group with severe hepatic impairment than in the control group (Table 12).
GM AUCinf was comparable with an increase of factor ~1.1 in the group with mild hepatic impairment (116.80 h*ng/mL), and factor ~1.0 in the group with moderate hepatic impairment (107.64 h*ng/mL), and ~1.9-fold higher in the group with severe impairment (197.53 h*ng/ml) (Table 12).
Compared to the normal hepatic group function, the 90% CIs did not contain unity, ie, the ratio of 1, for the estimates of Cmax and AUCinf for severe group and Cmax for the moderate group, thus suggesting that the differences were relevant (Table 12). In fact 90% CIs are completely shifted above the usual bioequivalence limits (0.80-1.25).
GM T½ were similar in normal hepatic function, mild impairment, and moderate impairment. A considerably lower T½ was observed in the severe hepatic impairment group compared to the other groups. (Table 8).
Median Tmax was comparable across all hepatic function groups (Tmax ranged from 0.23 h to 1.92 h) (Table 8).
The GM for apparent clearance CL/F of E4 was comparable for the groups with normal hepatic function and mild and moderate hepatic impairment, and approximately half in the group with severe hepatic impairment (191.77 L/h) compared to the normal hepatic function group 101.25 L/h) (Table 8).
The GM Vz/F was comparable across hepatic function groups except for the severe hepatic impairment group, which had a GM Vz/F of 1201.4 L compared to 5996.6 L in the normal hepatic function group.
The 2-way ANOVA confirms that hepatic function is a variable affecting the PK parameters Cmax and AUCinf whereas BMI has no effect. (Table 13).

### 3.2.2 E4-3-Glucuronide Pharmacokinetics

GM Cmax was comparable across all hepatic function groups (Table 9).
GM AUCinf showed a slight decrease in moderate and severe hepatic impairment groups when compared to the group with normal hepatic function, whereas GM AUC0-24 was comparable across the groups (Table 9).
Median Tmax appeared to be comparable across all but 1 hepatic function groups, for the moderate hepatic impairment group it was 1 h compared to ~0.5 h in the 3 other groups (Table 9).
GM T½ was reduced in the groups of moderate and severe hepatic impairment when compared to the normal hepatic function group and increased in the group with mild hepatic impairment (Table 9).

### 3.2.3 E4-16-Glucuronide Pharmacokinetics

GM Cmax was increased in all groups with hepatic impairment, most pronounced in the group with severe impairment (Table 10).
GM AUCinf and GM AUC0-24 showed an increase in the hepatic impairment groups, most pronounced in the group with severe impairment (Table 10).
Median Tmax appeared to be comparable across all hepatic function groups ((Table 10).
GM T½ appeared to be similar in the groups with normal hepatic function, mild or moderate hepatic impairment, and reduced in the group with severe hepatic impairment.

### 4. Safety Evaluation

### 4.1 Extent of Exposure

All subjects in all groups received 20 mg E4 as described in the protocol.

### 4.2 Adverse Events

### 4.2.1 Brief Summary of Adverse Events

During this study, 9 subjects (28.1%) had at least 1 TEAE: 2 subjects each in the groups of normal hepatic function and mild hepatic impairment; 4 subjects in the group of moderate hepatic impairment; and 1 subject in the group of severe hepatic impairment.

There were no subjects with SAEs.

Six subjects (18.8%) had TEAEs considered of mild severity, and 3 (9.4%) had TEAEs of moderate severity. Eight subjects (25.0%) had TEAEs that were considered by the investigator not related to the study drug, and 1 subject (3.1%) had TEAEs that were considered related to the study drug (Table 14).

The most frequently reported TEAEs were diarrhea for 3 subjects (9.4%), followed by 2 (6.3%) each of headache back pain, hypertension.

TABLE 12
Overall Summary of Adverse Events (Safety Set)

| | Normal | Mild | Moderate | Severe | Total |
|---|---|---|---|---|---|
| | (N = 8) | (N = 8) | (N = 8) | (N = 8) | (N = 32) |
| | n (%) | n (%) | n (%) | n (%) | n (%) |
| Subjects with at least one | 2 (25.0) | 2 (25.0) | 4 (50.0) | 1 (12.5) | 9 (28.1) |
| AE | | | | | |
| Subjects with at least one | 2 (25.0) | 2 (25.0) | 4 (50.0) | 1 (12.5) | 9 (28.1) |
| TEAE | | | | | |
| Subjects with at least one | 0 | 0 | 0 | 0 | 0 |
| SAE | | | | | |
| Subjects with at least one | 0 | 0 | 0 | 0 | 0 |
| TEAE leading to study | | | | | |
| drug discontinuation | | | | | |
| Subjects with TEAEs by | | | | | |
| maximum severity | | | | | |
| Mild | 0 | 2 (25.0) | 3 (37.5) | 1 (12.5) | 6 (18.8) |
| Moderate | 2 (25.0) | 0 | 1 (12.5) | 0 | 3 (9.4) |
| Subjects by maximum | | | | | |
| relationship to study drug | | | | | |
| Not related | 2 (25.0) | 2 (25.0) | 3 (37.5) | 1 (12.5) | 8 (25.0) |
| Related | 0 | 0 | 1 (12.5) | 0 | 1 (3.1) |

AE = adverse event;
N = total number of subjects;
n = number of assessed subjects;
SAE = serious adverse event;
TEAE = treatment-emergent adverse events
Note:
Subjects are counted only once under the category of their most severe event.
Note:
Subjects are counted only once under the category of their most drug-related event, regardless of how many drug-related events they reported

### 4.2.2 Display of Adverse Events

Table 15 shows the summary of TEAEs by SOC and preferred term. Table 16 presents the summary of TEAEs by severity.

TABLE 13
Summary of Treatment-Emergent Adverse Events by System
Organ Class and Preferred Term (Safety Set)

| | Normal | Mild | Moderate | Severe | Total |
|---|---|---|---|---|---|
| System Organ Class | (N = 8) | (N = 8) | (N = 8) | (N = 8) | (N = 32) |
| Preferred Term | n (%) | n (%) | n (%) | n (%) | n (%) |
| Total Subjects with at Least One | 2 (25.0) | 2 (25.0) | 4 (50.0) | 1 (12.5) | 9 (28.1) |
| TEAE | | | | | |
| Gastrointestinal disorders | 2 (25.0) | 0 (0.0) | 2 (25.0) | 0 (0.0) | 4 (12.5) |
| Diarrhea | 1 (12.5) | 0 (0.0) | 2 (25.0) | 0 (0.0) | 3 (9.4) |
| Pancreatitis | 1 (12.5) | 0 (0.0) | 0 (0.0) | 0 (0.0) | 1 (3.1) |
| General disorders and | 0 (0.0) | 0 (0.0) | 1 (12.5) | 0 (0.0) | 1 (3.1) |
| administration site conditions | | | | | |
| Pyrexia | 0 (0.0) | 0 (0.0) | 1 (12.5) | 0 (0.0) | 1 (3.1) |
| Musculoskeletal and connective | 1 (12.5) | 1 (12.5) | 1 (12.5) | 1 (12.5) | 4 (12.5) |
| tissue disorders | | | | | |
| Back pain | 1 (12.5) | 0 (0.0) | 0 (0.0) | 1 (12.5) | 2 (6.3) |
| Haemarthrosis | 0 (0.0) | 1 (12.5) | 0 (0.0) | 0 (0.0) | 1 (3.1) |
| Pain in extremity | 0 (0.0) | 0 (0.0) | 1 (12.5) | 0 (0.0) | 1 (3.1) |
| Nervous system disorders | 0 (0.0) | 0 (0.0) | 2 (25.0) | 0 (0.0) | 2 (6.3) |
| Headache | 0 (0.0) | 0 (0.0) | 2 (25.0) | 0 (0.0) | 2 (6.3) |
| Renal and urinary disorders | 0 (0.0) | 0 (0.0) | 0 (0.0) | 1 (12.5) | 1 (3.1) |
| Renal impairment | 0 (0.0) | 0 (0.0) | 0 (0.0) | 1 (12.5) | 1 (3.1) |
| Vascular disorders | 0 (0.0) | 2 (25.0) | 0 (0.0) | 0 (0.0) | 2 (6.3) |
| Hypertension | 0 (0.0) | 2 (25.0) | 0 (0.0) | 0 (0.0) | 2 (6.3) |

N = total number of subjects;
n = number of assessed subjects;
TEAE = treatment-emergent adverse events
Note:
Coded using the Medical Dictionary for Regulatory Activities version 22.1
Note:
n - Subjects are counted only once within each system organ class and preferred term.

TABLE 14
Summary of Treatment-Emergent Adverse Events by Severity (Safety Set)

| | | Normal | Mild | Moderate | Severe | Total |
|---|---|---|---|---|---|---|
| System Organ Class | | (N = 8) | (N = 8) | (N = 8) | (N = 8) | (N = 32) |
| Preferred Term | Severity | n (%) | n (%) | n (%) | n (%) | n (%) |
| Gastrointestinal | Mild | 0 (0.0) | 0 (0.0) | 2 (25.0) | 0 (0.0) | 2 (6.3) |
| disorders | Moderate | 2 (25.0) | 0 (0.0) | 0 (0.0) | 0 (0.0) | 2 (6.3) |
| Diarrhea | Mild | 0 (0.0) | 0 (0.0) | 2 (25.0) | 0 (0.0) | 2 (6.3) |
| | Moderate | 1 (12.5) | 0 (0.0) | 0 (0.0) | 0 (0.0) | 1 (3.1) |
| Pancreatitis | Moderate | 1 (12.5) | 0 (0.0) | 0 (0.0) | 0 (0.0) | 1 (3.1) |
| General disorders and | Mild | 0 (0.0) | 0 (0.0) | 1 (12.5) | 0 (0.0) | 1 (3.1) |
| administration site | | | | | | |
| conditions | | | | | | |
| Pyrexia | Mild | 0 (0.0) | 0 (0.0) | 1 (12.5) | 0 (0.0) | 1 (3.1) |
| Musculoskeletal and | Mild | 0 (0.0) | 1 (12.5) | 1 (12.5) | 1 (12.5) | 3 (9.4) |
| connective tissue | Moderate | 1 (12.5) | 0 (0.0) | 0 (0.0) | 0 (0.0) | 1 (3.1) |
| disorders | | | | | | |
| Back pain | Mild | 0 (0.0) | 0 (0.0) | 0 (0.0) | 1 (12.5) | 1 (3.1) |
| | Moderate | 1 (12.5) | 0 (0.0) | 0 (0.0) | 0 (0.0) | 1 (3.1) |
| Haemarthrosis | Mild | 0 (0.0) | 1 (12.5) | 0 (0.0) | 0 (0.0) | 1 (3.1) |
| Pain in extremity | Mild | 0 (0.0) | 0 (0.0) | 1 (12.5) | 0 (0.0) | 1 (3.1) |
| Nervous system | Mild | 0 (0.0) | 0 (0.0) | 1 (12.5) | 0 (0.0) | 1 (3.1) |
| disorders | Moderate | 0 (0.0) | 0 (0.0) | 1 (12.5) | 0 (0.0) | 1 (3.1) |
| Headache | Mild | 0 (0.0) | 0 (0.0) | 1 (12.5) | 0 (0.0) | 1 (3.1) |
| | Moderate | 0 (0.0) | 0 (0.0) | 1 (12.5) | 0 (0.0) | 1 (3.1) |
| Renal and urinary | Mild | 0 (0.0) | 0 (0.0) | 0 (0.0) | 1 (12.5) | 1 (3.1) |
| disorders | | | | | | |
| Renal impairment | Mild | 0 (0.0) | 0 (0.0) | 0 (0.0) | 1 (12.5) | 1 (3.1) |
| Vascular disorders | Mild | 0 (0.0) | 2 (25.0) | 0 (0.0) | 0 (0.0) | 2 (6.3) |
| Hypertension | Mild | 0 (0.0) | 2 (25.0) | 0 (0.0) | 0 (0.0) | 2 (6.3) |

N = number of subjects exposed;
n = number of subjects that experienced the AEs
Note:
Coded using the Medical Dictionary for Regulatory Activities version 22.1
Note:
n—Subjects are counted only once under the category of their most severe event within each system organ class and preferred term.

### 4.2.3 Analysis of Adverse Events

AE data were analyzed by frequency, severity, outcome, and relationship to the study drug. No additional analyses of AEs were performed. The most common TEAE was diarrhea (gastrointestinal disorders) with 3 subjects. Additionally, back pain (musculoskeletal and connective tissue disorders), headache (nervous system disorders), and hypertension (vascular disorders) were each reported by 2 subjects (Table 15).

Eleven TEAEs were considered of mild intensity and 4 of moderate intensity. Diarrhea was reported by 1 subject in the normal group and by 2 subjects in the moderate group. The events were considered of moderate intensity for the subject in the normal group and mild for the subjects in the moderate group. Headache was reported by 2 subjects in the moderate group; 1 of them moderate and the other was of mild intensity. Back pain was reported by 1 subject in the normal group and by 1 subject in the severe group. The events were considered of moderate intensity for the subject in the normal group and mild for the subject in the moderate group. Hypertension was recorded for 2 subjects in the mild group and was considered of mild intensity in both subjects (Table 16).

All TEAEs reported during this study were considered not related to the study drug with the exception of 1 TEAE of diarrhea reported by 1 subject in the moderate group and considered of mild intensity.

### 4.3 Deaths, Other Serious Adverse Events, and Other Significant Adverse Events

No deaths, SAEs, or other significant AEs occurred during this study. There were no discontinuations from the study because of TEAEs.

### 4.3.1 Evaluation of Each Laboratory Parameter

4.3.1.1 Laboratory Values over Time

The summaries of laboratory values, their changes from baseline, and shifts from baseline did not show deviations or trends which would raise safety concerns for the parameters of hematology or clinical chemistry after the administration of E4 in subjects with mild, moderate, or severe hepatic impairment, and in subjects with normal hepatic function.

### 4.3.1.2 Individual Subject Changes

The majority of laboratory values were within normal ranges during the course of the clinical study (Screening, Day −1, and Follow-up). Many of the individual deviations from normal ranges of the assessed laboratory parameters during the study were already present at Screening or baseline and could be attributed to the overall health condition or the underlying disease(s) of the study population. Deviations from normal laboratory ranges in individual subjects in hematology, clinical chemistry, coagulation and urinalysis were qualified as not clinically significant due to minor out-of-range values; except 2 subjects who had out-of-range values judged as clinically significant.

### 4.3.1.3 Individual Clinically Significant Abnormalities

Subject 3022 in the group with normal hepatic function, who had no history of gall bladder infection or pancreatitis, had clinically significant elevations of levels of amylase on Day 3 and Day 5, of lipase on Day 9 and Follow-up, and furthermore at Follow-up of alanine aminotransferase, alkaline phosphatase, aspartate aminotransferase, gamma glutamyl transferase, and lactate dehydrogenase, which was diagnosed as a subclinical pancreatitis. The subject had no symptoms throughout the event, received no treatment, and was not hospitalized. The subject was monitored until all parameters returned to near baseline levels (judged as not clinically significant) within 9 days after Follow-up.

For Subject 3028 in the group with severely impaired hepatic function isolated instances of elevated levels for creatinine and urea were measured on Day −1.

### 4.4 Vital Signs, Physical Findings, and Other Observations Related to Safety

### 4.4.1 Vital Signs

Although several individual changes from baseline were observed, blood pressure, pulse rate, respiratory rate, and body temperature showed no trends or clinically relevant changes.

### 4.4.2 Physical Examination

All but 1 abnormalities observed were considered to be of no clinical relevance. One subject in the mild hepatic impairment group had a suffusion (diffuse hematoma) (MedDRA preferred term: Haemarthrosis) in the left elbow on Day 3 that was considered clinically significant and reported as TEAE. The event was of mild severity and deemed by the investigator not related to the study drug. The suffusion resolved without additional treatment.

### 4.5 Safety Conclusions

A single oral dose of 20 mg E4 monohydrate in female subjects with normal hepatic function, mild, moderate, or severe hepatic impairment was safe and well tolerated.
No deaths, SAEs, or other significant AEs occurred during this study.
The most common TEAE was diarrhea (gastrointestinal disorders) with 3 subjects. Diarrhea was reported by 1 subject in the group with normal hepatic function and by 2 subjects in the group with moderate hepatic impairment. The events were considered of moderate intensity for the subject in the group with normal hepatic function and mild for the subjects in the group with moderate hepatic impairment.
All TEAEs reported during this study were considered not related to the study drug with the exception of 1 TEAE of diarrhea reported by 1 subject in the group with moderate hepatic impairment and considered of mild intensity.
The summaries of laboratory values, their changes from baseline, and shifts from baseline did not show deviations or trends which would raise safety concerns for the parameters of hematology or clinical chemistry after the administration of E4 monohydrate in subjects with mild, moderate, or severe hepatic impairment, and in subjects with normal hepatic function.
The results of the physical examination were generally unremarkable.

### 5. Discussion and Overall Conclusions

This was a Phase 1, multi-center, open-label, PK, and safety study of a single oral dose of 20 mg E4 monohydrate in female subjects with different degrees of hepatic impairment and subjects with normal hepatic function. Previous studies on absorption, distribution, metabolism, and excretion suggest hepatic involvement, which is a well-known pathway for other estrogens.

### 5.1 Pharmacokinetics

E4 was readily absorbed and attained Cmax within the first 2 h after oral administration, independent from the hepatic function status. The mean Cmax ranged from ~24 ng/mL in subjects with normal hepatic function, over ~38 ng/mL in subjects with mild hepatic impairment and ~52 ng/mL in subjects with moderate hepatic impairment to ~117 ng/ml in subjects with severe hepatic impairment.

The comparison of Cmax in moderate and severe hepatic impaired subjects versus normal hepatic function showed a relevant difference (1.896-fold, 90% CI: 1.132-3.178 and 5.359-fold, 90% CI: 3.198-8.981, respectively), while this was not the case for the comparison of mild hepatic impairment versus normal hepatic function (1.661-fold, 90% CI: 0.991-2.783).

The distribution and reabsorption phase eventually accounted for an increase of AUC0 24 according to the degree of hepatic dysfunction, with GMs of 69.7554 h*ng/mL, 88.7526 h*ng/mL, and 179.0494 h*ng/ml in subjects with mild, moderate, and severe hepatic impairment, respectively, compared to 60.4146 h*ng/ml in subjects with normal hepatic function.

For AUCinf these differences were not significant in mild and moderate versus normal (1.120-fold, 90% CI: 0.808-1.552 and 1.032-fold, 90% CI: 0.745-1.431, respectively), but significant in severe versus normal (1.894-fold, 90% CI: 1.367-2.625).

The elimination phase of the concentration-time profiles revealed a decrease of the half-life of E4 ordered along the degree of hepatic dysfunction, with a GM T½ of 21.67 h in subjects with normal hepatic function, followed by 20.07 h in subjects with mild hepatic impairment, 14.79 h in subjects with moderate hepatic impairment, and 8.22 h in subjects with severe hepatic impairment. Correspondingly, the last measurable concentration of E4 (Clast) was attained 192 h post-dose (the last measured timepoint) in subjects with normal hepatic function or with mild hepatic impairment, 144 h in subjects with moderate hepatic impairment, and 168 h in subjects with severe hepatic impairment.

Current results are in line with the influence of hepatic impairment on distribution and reabsorption cycles, especially with the influence of severe hepatic impairment on enterohepatic recycling.

In particular, E4 plasma levels at 24 h after administration were in the same range among the different hepatic function groups, which could then result in similar accumulation following a once-a-day multiple dose regimen.

### 5.2 Safety

In this study, a single oral dose of 20 mg E4 monohydrate administered in female subjects with normal hepatic function, mild, moderate, or severe hepatic impairment was safe and well tolerated.

The most common TEAE was diarrhea (gastrointestinal disorders) with 3 subjects. Diarrhea was reported by 1 subject in the normal group and by 2 subjects in the group with moderate hepatic impairment. The events were considered of moderate intensity for the subject in the normal group and mild for the subjects in the group with moderate hepatic impairment.

All TEAEs reported during this study were considered not related to the study drug with the exception of 1 TEAE of diarrhea reported by 1 subject in the group of moderate hepatic impairment and considered of mild intensity.

The summaries of laboratory values, their changes from baseline, and shifts from baseline did not show deviations or trends which would raise safety concerns for the parameters of hematology or clinical chemistry after the administration of E4 in subjects with mild, moderate, or severe hepatic impairment, and in subjects with normal hepatic function.

The results of the physical examination were generally unremarkable.

### 5.3 Conclusions

### 5.3.1 Pharmacokinetics

GM Cmax of E4 after a single dose of 20 mg was significantly increased in subjects with moderate (~1.9-fold, 90% CI: 1.132-3.178) and severe hepatic impairment (~5.4 fold, 90% CI: 3.198-8.981) but not between subjects with mild hepatic impairment and subjects with normal hepatic function (~1.7-fold and 90% CI: 0.991-2.783).
The peak exposure for the metabolites correlated with the degree of hepatic impairment, but the change is negligible due to their low potency and small proportion as pharmacological compound.
GM AUCinf of E4 was comparable in the group with mild hepatic impairment (~1.1 fold, 90% CI 0.808-1.552) and in the group with moderate hepatic impairment (~1.0-fold, 90% CI 0.745-1.431), and ~1.9-fold higher (90% CI 1.367 2.625) in the group with severe hepatic impairment when compared to normal hepatic function.
GM AUCinf of E4-3-glucuronide appeared slightly reduced in moderate and severe hepatic impairment compared to normal hepatic function, however the results show a lot of variability. For E4-16-glucuronide, GM AUCinf showed an increase in the hepatic impairment groups, most pronounced in the severe group.
GM T½ overall appeared to shorten for E4 and metabolites with the degree of hepatic impairment, although variability is high.

### 5.3.2 Safety

A single oral dose of 20 mg E4 monohydrate in female subjects with normal hepatic function, mild, moderate, or severe hepatic impairment was safe and well tolerated.

The most common TEAE was diarrhea (gastrointestinal disorders). Diarrhea was reported by 3 subjects: 1 subject in the normal group and by 2 subjects in the group with moderate hepatic impairment. The events were considered of moderate intensity for the subject in the normal group and mild for the subjects in the group with moderate hepatic impairment.

All TEAEs reported during this study were considered not related to the study drug with the exception of 1 TEAE of diarrhea reported by 1 subject in the group of moderate hepatic impairment and considered of mild intensity.

Vital signs, laboratory values, and physical examination were unremarkable.

### Example 2. A Dose-Finding Study to Select the Daily Oral Dose of Estetrol (E4) for the Treatment of Vasomotor Symptoms in Post-Menopausal Women

### Study Enrolment and Duration:

Enrolment was approximately 18 months. Individual subject participation was up to 27 weeks: up to 6 weeks pre-screening and washout, up to 4 weeks screening and run-in period, up to 91 days (13 weeks) of E4 monohydrate or placebo treatment followed by 2 weeks (14 days) of progestin therapy and a Follow up visit 1 week after completion of progestin therapy in non-hysterectomied subjects only.

### Primary Efficacy Objective:

To define the minimum effective dose (MED) of the oral dose of E4 by evaluating changes in frequency and in severity of moderate to severe vasomotor symptoms (VMS).

### Methodology:

This was a prospective, multicentre, randomised, placebo-controlled, double-blinded, dose-finding study.

### Subject Population:

Eligible subjects were hysterectomied and non-hysterectomied post-menopausal women aged 40 to 65 years, inclusive, presenting at least 7 moderate to severe hot flushes/day or at least 50 moderate to severe hot flushes/week.

### Diagnosis and Inclusion Criteria:

The subjects have met all of the following inclusion criteria at the randomization visit. These criteria were assessed during the screening period:
1. Women aged 40 to 65 years, inclusive, presenting at least 7 moderate to severe hot flushes/day or at least 50 moderate to severe hot flushes/week in the week preceding randomization.
2. Body Mass Index (BMI) between 18.0 and 35.0 kg/m², inclusive.
3. Post-menopausal status defined as levels of follicle stimulating hormone (FSH)>40 IU/L and:
   amenorrhoea for at least 12 consecutive months or,
   amenorrhoea for at least 6 months with estradiol (E2)<20 μg/mL or,
   at least 6 weeks post-surgical bilateral oophorectomy with or without hysterectomy with a copy of the pathology report or a statement on letterhead from the subject's physician documenting both ovaries were removed is required.
4. For non-hysterectomied women: intact uterus with bi-layer endometrial thickness≤5 mm on TVUS.
5. Negative pregnancy test.
6. Good physical and mental health, in the judgement of the Principal Investigator (PI), on the basis of medical, surgical and gynaecological history, physical examination, gynaecological examination, clinical laboratory, and vital signs.
7. Subject has provided signed and dated written informed consent before admission to the study.
8. Subject is able to understand and comply with the protocol requirements, instructions, and protocol-stated restrictions.

### Exclusion Criteria:

Potential study subjects were excluded if one of the following exclusion criteria was present at the randomization visit. These criteria were assessed during the screening period:
1. For non-hysterectomied women: uterine disease or medical condition including:
   a. Bi-layer endometrial thickness>5 mm as determined by TVUS;
   b. Presence of fibroid(s) that obscure(s) evaluation of endometrium by TVUS;
   c. History or presence of uterine cancer;
   d. Presence of endometrial hyperplasia;
   e. Presence of an endometrial polyp with hyperplastic or malignant epithelium.
2. Undiagnosed vaginal bleeding in the last 12 months.
3. Any history of malignancy with the exception of basal cell (excluded if within the prior 2 years) or squamous cell (excluded if within the prior one year) carcinoma of the skin. Any clinically significant findings at the breast examination and/or on mammography suspicious of breast malignancy that would require additional clinical testing to rule out breast cancer (however, simple cysts confirmed by ultrasound were allowed). Note: A screening mammogram was required unless the subject had a written documentation of a mammogram performed within the last 9 months.
4. Abnormal cervical Pap smear in non-hysterectomied subjects (written documentation of prior test within 18 months or test at screening exam) with evidence of cervical dysplasia greater than low grade squamous intraepithelial lesion (LSIL). Women with a diagnosis of atypical squamous cells of undetermined significance (ASCUS) were enrolled.
5. Systolic blood pressure (BP) outside the range 90 to 140 mmHg, diastolic BP outside the range 60 to 90 mmHg, and/or heart rate outside the range 40 to 100 bpm. Subjects with mild to moderate hypertension who were controlled on a stable antihypertension regimen were enrolled if they met the inclusion/exclusion criteria.
6. Any clinically significant abnormality identified on the screening 12-lead ECG.
7. History of venous or arterial thromboembolic disease (e.g., deep vein thrombosis, pulmonary embolism, stroke, myocardial infarction, angina pectoris, etc.), history of known coagulopathy or abnormal coagulation factors.
8. Diabetes mellitus with poor glycaemic control in the last 6 months assessed by laboratory values of glucose outside the normal ranges and glycated haemoglobin above 7%.
9. Dyslipoproteinaemia predisposes the subject to atherosclerotic cardiovascular disease (ASCVD). If a subject had a 10 years ASCVD score≥5% as calculated using the ASCVD risk estimator (ACC/AHA Cardiovascular risk assessment guideline, 2013), she was not be included in the trial. In all cases, LDL cholesterol level≥190 mg/dL or triglycerides plasma level>400 mg/dL were exclusionary.
   If a subject was receiving a lipid-lowering therapy, her treatment had to be on a stable dose for at least 1 month before screening and the same eligibility criteria had to be used.
10. Smoking >10 cigarettes/day or use of >1 ml/day of nicotine containing liquid for electronic cigarette.
11. Presence or history of gallbladder disease, unless cholecystectomy had been performed.
12. Systemic lupus erythematosus.
13. Multiple sclerosis.
14. Acute or chronic liver disease.
15. Acute or chronic renal impairment, including severe renal impairment.
16. Uncontrolled thyroid disorders.
17. Subject had a history of major depression or post-traumatic stress disorder (PTSD) within 2 years, OR a history of other major psychiatric disorder at any time (e.g., schizophrenia, bipolar disorder, etc.).
18. Use of estrogen or progestin containing drug(s). A washout period is required before the Run-in Period in case of use of:
   a. Vaginal hormonal products (rings, creams, gels): washout of at least 4 weeks;
   b. Transdermal estrogen or estrogen/progestin: washout of at least 4 weeks;
   c. Oral estrogen and/or progestin: washout of at least 4 weeks;
   d. Intrauterine progestin therapy: washout of at least 4 weeks;
   Current users of progestin implants or estrogen alone injectable drug therapy were not allowed to participate unless the treatment was stopped more than 3 months ago. Current users of estrogen pellet therapy or progestin injectable drug therapy were not allowed to participate unless the treatment was stopped more than 6 months ago.
19. Use of non-hormonal treatments to reduce hot flushes. A washout period of 1 week was required before the Run-in Period in the case of use of non-hormonal prescription and over-the-counter (OTC) treatments for hot flushes (such as anti-depressants paroxetine, escitalopram, venlafaxine, desvenlafaxine, and clonidine; or phytoestrogens, black cohosh, etc.). If one of these treatments was concomitantly taken with an estrogen or progestin-containing drug, washout periods could be combined and did not have to be sequential.
20. Use of medication that may affect the outcome of the VMS endpoints within 28 days before the Run-in Period. This included (but was not limited to): SSRIs [selective serotonin reuptake inhibitors], SNRIs [serotonin and norepinephrine reuptake inhibitors], dopaminergic or antidopaminergic drugs, or gabapentin.
21. History or presence of allergy to the investigational product or drugs of this class, or history of drug or other allergy that, in the opinion of the Investigator contraindicated subject participation.
22. History or presence of allergy or intolerance to any component of the investigational product.
23. History of alcohol or substance abuse or dependence in the 12 months as determined by the Investigator, i.e. subject consumed excessive alcohol, abused drugs, or had a condition that could compromise the subject's ability to comply with study requirements in the Investigator's opinion.
24. Sponsor or Contract Research Organization (CRO) employees, or personnel in the department of the Investigator and relatives affiliated with this study.
25. Subjects with porphyria and subjects with known or suspected history of a clinically significant systemic disease, unstable medical disorders, life-threatening disease or current malignancies that would pose a risk to the subject in the opinion of the Investigator.
26. Participation in another investigational drug clinical study within 1 month (30 days) or had received an investigational drug within the last 3 months (90 days).
27. Was judged by the Investigator to be unsuitable for any reason.

### Number of Subjects:

### Intention-to-Treat Principle

This principle asserts that the effect of a treatment policy can be best assessed by evaluating on the basis of the intention to treat a subject (i.e. the planned treatment regimen) rather than the actual treatment given. It has the consequence that subjects allocated to a treatment group should be followed up, assessed and analysed as members of that group irrespective of their compliance to the planned course of treatment.

Further, the intention-to-treat principle implies that the primary analysis should include all randomised subjects. Preservation of the initial randomisation in analysis is important in preventing bias and in providing a secure foundation for statistical tests. In many clinical trials the use of the full analysis set provides a conservative strategy. Under many circumstances it may also provide estimates of treatment effects which are more likely to mirror those observed in subsequent practice.

In the present study, the Intention-To-Treat group included a total of 257 patients.

Subjects were randomly allocated to one of the 5 treatment arms in a 1:1:1:1:1 ratio. The randomisation was stratified by centre.

### Study Visits:

| | | Notation as used throughout all | |
|---|---|---|---|
| Week | Visit | tables, listings and figures | Study part |
| Week −7 to −4 | Visit 1 (Pre-Screening) | V1 | Screening |
| Week −4 to −3 | Visit 1a (Screening) | V1a | Screening |
| Week −1 | Visit 2 (Baseline) | V2 | Randomisation |
| Week 5 | Visit 3 | V3 | Treatment period |
| Week 13 | Visit 4 (EOT) | V4 | End of treatment |
| Week 16 | Visit 5 (EOS) | V5 | End of study |

### Test Product and Reference Therapy, Dose, and Mode of Administration

All treatments (Estetrol, hereinafter E4 as monohydrate, [2.5 mg, 5 mg, 10 mg, 15 mg] capsule) were administered once daily (QD) per os for at least 12 consecutive weeks until the last biological assessments (Day 91 maximum) had been performed.

Placebo, 1 capsule administered QD per os for at least 12 consecutive weeks until the last biological assessments (Day 90 maximum) have been performed.

If during the course of the trial, a double layer endometrial thickness≥15 mm was detected on TVUS and/or abnormal uterine bleeding (in the judgement of the gynecologist in light of the estrogen therapy) was reported by a non-hysterectomied woman, she underwent an endometrial biopsy and was treated with progestin (10 mg dydrogesterone) QD until end of Week 11 in a sequential way (i.e., a 14 day progestin treatment period followed by a 14 day progestin treatment pause) in addition to the E4/placebo treatment. If the endometrial biopsy showed endometrial hyperplasia, the subject participation was immediately stopped and the treatment of hyperplasia was performed as per local guidelines. If an abnormal uterine bleeding occurred again after a first normal endometrial biopsy, a thorough gynaecological examination and a TVUS were performed. If necessary in the judgment of the gynecologist, a second endometrial biopsy was performed.

After the E4 monohydrate or placebo treatment period, all non-hysterectomied subjects (including those having received the progestin previously) received progestin therapy for 14 days with 10 mg dydrogesterone QD.

### Results

### A. Vasomotors Parameters for Each of the 5 Treatment Groups

a. VMS Frequency
i. Absolute Change (Mean Change from Baseline) in Weekly Frequency of Moderate to Severe VMS

### a) Week-by-Week for Each Group

| | 2.5 mg E4 | | 5 mg E4 | | 10 mg E4 | | 15 mg E4 | | | |
|---|---|---|---|---|---|---|---|---|---|---|
| | monohydrate | | monohydrate | | monohydrate | | monohydrate | | Placebo | |
| Week | Mean | SD | Mean | SD | Mean | SD | Mean | SD | Mean | SD |
| 0 | 0.00 | 0.00 | 0.00 | 0.00 | 0.00 | 0.00 | 0.00 | 0.00 | 0.00 | 0.00 |
| 1 | −17.28 | 19.71 | −15.04 | 17.36 | −13.37 | 19.59 | −14.86 | 16.45 | −16.65 | 16.24 |
| 2 | −25.10 | 23.91 | −21.68 | 19.26 | −23.47 | 24.74 | −29.19 | 20.25 | −27.38 | 23.16 |
| 3 | −32.46 | 26.15 | −24.38 | 21.36 | −31.34 | 25.21 | −36.30 | 23.23 | −30.16 | 23.09 |
| 4 | −35.89 | 31.57 | −27.57 | 22.47 | −36.38 | 22.62 | −41.43 | 21.60 | −32.94 | 23.14 |
| 5 | −37.16 | 33.27 | −30.70 | 23.05 | −39.07 | 21.55 | −44.34 | 20.95 | −34.70 | 23.01 |
| 6 | −39.98 | 35.44 | −38.02 | 22.13 | −42.56 | 21.18 | −46.58 | 19.83 | −37.18 | 21.25 |
| 7 | −42.44 | 36.96 | −38.23 | 21.73 | −43.30 | 22.18 | −48.51 | 19.34 | −38.55 | 21.85 |
| 8 | −43.62 | 38.43 | −38.55 | 23.54 | −44.46 | 23.30 | −48.35 | 19.75 | −38.13 | 21.69 |
| 9 | −45.19 | 36.91 | −39.34 | 23.51 | −45.19 | 24.22 | −49.62 | 18.97 | −39.25 | 22.74 |
| 10 | −45.54 | 37.93 | −39.83 | 23.79 | −45.93 | 23.58 | −48.88 | 20.04 | −41.12 | 21.82 |
| 11 | −44.74 | 39.30 | −41.46 | 23.59 | −45.90 | 23.57 | −49.92 | 19.42 | −42.50 | 22.68 |
| 12 | −45.04 | 38.91 | −40.60 | 24.37 | −47.21 | 22.87 | −50.94 | 18.38 | −42.97 | 22.31 |

In order to analyse the data recorded in this study, treatment groups have been compared using an ANCOVA (analysis of covariance) with respect to the change in weekly frequency of moderate to severe VMS from baseline to weeks 4 and 12. The ANCOVA model includes treatment (“trt1”) and study centre (“SITEPOOL”) as a fixed effect and baseline (“base”) as a covariate.

### b) Covariate Significance

The following Table presents a comparison over all treatment groups.

| Analysis | | |
|---|---|---|
| Timepoint (N) | Covariate | p_value |
| 4 | trt1 | 0.0164 |
| 4 | base | <.0001 |
| 4 | SITEPOOL | 0.2214 |
| 12 | trt1 | 0.0384 |
| 12 | base | <.0001 |
| 12 | SITEPOOL | 0.2706 |

Upon review, it was considered that the effect due to the study centre was not very important, and a second ANCOVA was performed without accounting for study centres.

### Without Site Effect

| Analysis Timepoint (N) | Covariate | p_value |
|---|---|---|
| 4 | trt1 | 0.0130 |
| 4 | base | <.0001 |
| 12 | trt1 | 0.0254 |
| 12 | base | <.0001 |

c) The Table below presents mean change from baseline by week and treatment:

| Analysis | Planned | LS | Lower 95% | Upper 95% |
|---|---|---|---|---|
| Timepoint | treatment (mg | adjusted | confidence | confidence |
| (N) | E4 monohydrate) | mean | limit | limit |
| 4 | 0 | −33.7959277 | −40.006133 | −27.585722 |
| 4 | 2.5 | −32.2601479 | −38.742792 | −25.777504 |
| 4 | 5 | −27.4372730 | −34.135693 | −20.738853 |
| 4 | 10 | −35.3672857 | −41.716169 | −29.018402 |
| 4 | 15 | −43.5996056 | −50.256441 | −36.942770 |
| 12 | 0 | −43.7344395 | −50.150527 | −37.318352 |
| 12 | 2.5 | −40.1310220 | −46.828579 | −33.433465 |
| 12 | 5 | −40.6802008 | −47.600688 | −33.759714 |
| 12 | 10 | −45.8376026 | −52.396965 | −39.278240 |
| 12 | 15 | −53.8273074 | −60.704831 | −46.949784 |

### Without Site Effect

| Analysis | Planned | LS | Lower 95% | Upper 95% |
|---|---|---|---|---|
| Timepoint | treatment (mg | adjusted | confidence | confidence |
| (N) | E4 monohydrate) | mean | limit | limit |
| 4 | 0 | −33.6519764 | −39.821576 | −27.482377 |
| 4 | 2.5 | −32.6798943 | −39.056255 | −26.303533 |
| 4 | 5 | −27.8705668 | −34.540176 | −21.200958 |
| 4 | 10 | −35.8741722 | −42.156516 | −29.591828 |
| 4 | 15 | −44.3569892 | −50.965323 | −37.748656 |
| 12 | 0 | −43.9304437 | −50.292513 | −37.568374 |
| 12 | 2.5 | −40.6840423 | −47.259323 | −34.108761 |
| 12 | 5 | −41.0122585 | −47.889936 | −34.134581 |
| 12 | 10 | −46.5333871 | −53.011718 | −40.055056 |
| 12 | 15 | −54.9073270 | −61.721817 | −48.092837 |

All statistical tests are supported by presenting Least Square adjusted mean (LS adjusted mean: the group means after having controlled for a covariate; also referred to as marginal means or estimated marginal means) and 95% confidence intervals for the respective treatment effects. These LS adjusted means and Confidence Intervals are based on the statistical models used for the analysis.

The Confidence Intervals implies that if the same population is sampled on numerous occasions and interval estimates are made on each occasion, the resulting intervals would bracket the true population parameter in approximately 95% of the cases.

d) The Table below presents differences with placebo by week and treatment

| Analysis | Treatment | | | Lower 95% | Upper 95% | |
|---|---|---|---|---|---|---|
| Timepoint | (mg E4 | | LS adjusted | confidence | confidence | |
| (N) | monohydrate) | Placebo | mean | limit | limit | p_value |
| 4 | 2.5 | 0 | 1.535780 | −9.602669 | 12.674228 | 0.99183 |
| 4 | 5.0 | 0 | 6.358655 | −4.958071 | 17.675380 | 0.44910 |
| 4 | 10.0 | 0 | −1.571358 | −12.573355 | 9.430639 | 0.99067 |
| 4 | 15.0 | 0 | −9.803678 | −21.033229 | 1.425873 | 0.10653 |
| 12 | 2.5 | 0 | 3.603418 | −7.904295 | 15.111130 | 0.86103 |
| 12 | 5.0 | 0 | 3.054239 | −8.637660 | 14.746138 | 0.92091 |
| 12 | 10.0 | 0 | −2.103163 | −13.469900 | 9.263574 | 0.97581 |
| 12 | 15.0 | 0 | −10.092868 | −21.694703 | 1.508967 | 0.10838 |

### Without Site Effect

| Analysis | Treatment | | | Lower 95% | Upper 95% | |
|---|---|---|---|---|---|---|
| Timepoint | (mg E4 | | LS adjusted | confidence | confidence | |
| (N) | monohydrate) | placebo | mean | limit | limit | p_value |
| 4 | 2.5 | 0 | 0.972082 | −10.164048 | 12.108212 | 0.99860 |
| 4 | 5.0 | 0 | 5.781410 | −5.579628 | 17.142447 | 0.53888 |
| 4 | 10.0 | 0 | −2.222196 | −13.242056 | 8.797664 | 0.96728 |
| 4 | 15.0 | 0 | −10.705013 | −21.979289 | 0.569264 | 0.06834 |
| 12 | 2.5 | 0 | 3.246401 | −8.237137 | 14.729940 | 0.89864 |
| 12 | 5.0 | 0 | 2.918185 | −8.797276 | 14.633647 | 0.93259 |
| 12 | 10.0 | 0 | −2.602943 | −13.966584 | 8.760697 | 0.94911 |
| 12 | 15.0 | 0 | −10.976883 | −22.602878 | 0.649111 | 0.07057 |

From these Tables, it can be seen that the 15 mg estetrol monohydrate daily dose generates a near statistically significant difference with placebo at 4 weeks (p-value of 0.10653 in the first statistical analysis, and of 0.06834 in the analysis without pooled site) and at 12 weeks (p-value of 0.10838 in the first statistical analysis, and of 0.07057 in the analysis without pooled site).

ii. Relative Change (% from Baseline) in Weekly Frequency of Moderate to Severe VMS

### a) Week-by-Week for Each Group

| | 2.5 mg E4 | | 5 mg E4 | | 10 mg E4 | | 15 mg E4 | | | |
|---|---|---|---|---|---|---|---|---|---|---|
| | monohydrate | | monohydrate | | monohydrate | | monohydrate | | Placebo | |
| Week | Mean | SD | Mean | SD | Mean | SD | Mean | SD | Mean | SD |
| 0 | 0% | 0% | 0% | 0% | 0% | 0% | 0% | 0% | 0% | 0% |
| 1 | −23% | 23% | −23% | 28% | −21% | 29% | −22% | 33% | −26% | 25% |
| 2 | −34% | 27% | −33% | 29% | −37% | 37% | −46% | 34% | −41% | 35% |
| 3 | −44% | 29% | −38% | 33% | −48% | 39% | −57% | 34% | −45% | 33% |
| 4 | −49% | 33% | −43% | 34% | −55% | 35% | −67% | 30% | −49% | 32% |
| 5 | −50% | 33% | −47% | 34% | −60% | 33% | −72% | 28% | −52% | 32% |
| 6 | −54% | 36% | −57% | 31% | −65% | 33% | −76% | 26% | −56% | 30% |
| 7 | −57% | 36% | −57% | 30% | −66% | 34% | −79% | 25% | −58% | 30% |
| 8 | −58% | 38% | −57% | 32% | −68% | 35% | −79% | 26% | −58% | 31% |
| 9 | −61% | 36% | −59% | 32% | −69% | 36% | −81% | 24% | −60% | 31% |
| 10 | −61% | 37% | −60% | 32% | −70% | 35% | −80% | 27% | −63% | 30% |
| 11 | −60% | 39% | −63% | 31% | −70% | 34% | −82% | 25% | −65% | 31% |
| 12 | −61% | 38% | −62% | 32% | −72% | 33% | −84% | 23% | −65% | 30% |

From this table, it can be seen that the 15 mg estetrol monohydrate daily dose resulted in a reduction of over 80% in the frequency of moderate to severe VMS when compared to baseline.

### b) Covariate Significance

The Table below presents a comparison over all treatment groups.

| Analysis Timepoint (N) | Covariate | p_value |
|---|---|---|
| 4 | trt1 | 0.0147 |
| 4 | base | 0.3684 |
| 4 | SITEPOOL | 0.3236 |
| 12 | trt1 | 0.0100 |
| 12 | base | 0.1490 |
| 12 | SITEPOOL | 0.0958 |

### Without Site Effect

| Analysis Timepoint (N) | Covariate | p_value |
|---|---|---|
| 4 | trt1 | 0.0107 |
| 4 | base | 0.2264 |
| 12 | trt1 | 0.0065 |
| 12 | base | 0.0774 |

c) The Table below presents mean relative change (%) from baseline by week and treatment

| Analysis | Planned | LS | Lower 95% | Upper 95% |
|---|---|---|---|---|
| Timepoint | treatment (mg | adjusted | confidence | confidence |
| (N) | E4 monohydrate) | mean | limit | limit |
| 4 | 0 | −0.49080693 | −0.578953 | −0.402661 |
| 4 | 2.5 | −0.48482309 | −0.576836 | −0.392811 |
| 4 | 5 | −0.42247695 | −0.517552 | −0.327402 |
| 4 | 10 | −0.54456428 | −0.634678 | −0.454450 |
| 4 | 15 | −0.64721795 | −0.741703 | −0.552733 |
| 12 | 0 | −0.64237949 | −0.726371 | −0.558388 |
| 12 | 2.5 | −0.60565210 | −0.693328 | −0.517976 |
| 12 | 5 | −0.60259514 | −0.693189 | −0.512001 |
| 12 | 10 | −0.70041629 | −0.786283 | −0.614549 |
| 12 | 15 | −0.80089486 | −0.890927 | −0.710863 |

### Without Site Effect

| Analysis | Planned | LS | Lower 95% | Upper 95% |
|---|---|---|---|---|
| Timepoint | treatment (mg | adjusted | confidence | confidence |
| (N) | E4 monohydrate) | mean | limit | limit |
| 4 | 0 | −0.48986685 | −0.577115 | −0.402619 |
| 4 | 2.5 | −0.49532213 | −0.585494 | −0.405151 |
| 4 | 5 | −0.43132298 | −0.525642 | −0.337004 |
| 4 | 10 | −0.55503454 | −0.643877 | −0.466192 |
| 4 | 15 | −0.66014529 | −0.753597 | −0.566693 |
| 12 | 0 | −0.65107969 | −0.735113 | −0.567046 |
| 12 | 2.5 | −0.62210640 | −0.708956 | −0.535256 |
| 12 | 5 | −0.61436957 | −0.705214 | −0.523525 |
| 12 | 10 | −0.71732171 | −0.802891 | −0.631752 |
| 12 | 15 | −0.82338275 | −0.913392 | −0.733373 |

| Analysis | Treatment | | | Lower 95% | Upper 95% | |
|---|---|---|---|---|---|---|
| Timepoint | (mg E4 | | LS adjusted | confidence | confidence | |
| (N) | monohydrate) | Placebo | mean | limit | limit | p_value |
| 4 | 2.5 | 0 | 0.005984 | −0.152112 | 0.164079 | 0.99995 |
| 4 | 5.0 | 0 | 0.068330 | −0.092296 | 0.228956 | 0.68478 |
| 4 | 10.0 | 0 | −0.053757 | −0.209916 | 0.102401 | 0.81738 |
| 4 | 15.0 | 0 | −0.156411 | −0.315800 | 0.002977 | 0.05622 |
| 12 | 2.5 | 0 | 0.036727 | −0.113917 | 0.187372 | 0.93696 |
| 12 | 5.0 | 0 | 0.039784 | −0.113271 | 0.192840 | 0.92218 |
| 12 | 10.0 | 0 | −0.058037 | −0.206836 | 0.090762 | 0.74506 |
| 12 | 15.0 | 0 | −0.158515 | −0.310392 | −0.006639 | 0.03771 |

From this Table it can be seen that the 15 mg estetrol monohydrate daily dose generates a statistically significant difference with placebo at 12 weeks (p-value of 0.03771) and an almost statistically significant difference with placebo at 4 weeks (p-value of 0.05622).

It is particularly striking to observe the low p-values obtained for the 15 mg estetrol monohydrate dose by comparison to the elevated p-values obtained for the 10 mg estetrol monohydrate dose.

### Without Site Effect

| Analysis | Treatment | | | Lower 95% | Upper 95% | |
|---|---|---|---|---|---|---|
| Timepoint | (mg E4 | | LS adjusted | confidence | confidence | |
| (N) | monohydrate) | Placebo | mean | limit | limit | p_value |
| 4 | 2.5 | 0 | −0.005455 | −0.162937 | 0.152027 | 0.99996 |
| 4 | 5.0 | 0 | 0.058544 | −0.102119 | 0.219206 | 0.78717 |
| 4 | 10.0 | 0 | −0.065168 | −0.221006 | 0.090670 | 0.69797 |
| 4 | 15.0 | 0 | −0.170278 | −0.329714 | −0.010843 | 0.03206 |
| 12 | 2.5 | 0 | 0.028973 | −0.122708 | 0.180654 | 0.97301 |
| 12 | 5.0 | 0 | 0.036710 | −0.118034 | 0.191454 | 0.94271 |
| 12 | 10.0 | 0 | −0.066242 | −0.216339 | 0.083855 | 0.65761 |
| 12 | 15.0 | 0 | −0.172303 | −0.325866 | −0.018741 | 0.02210 |

From this Table it can be seen that in the statistical analysis not accounting for site effects, the 15 mg estetrol monohydrate daily dose generates a statistically significant difference with placebo at 4 weeks (p=0.03206) and at 12 weeks (p=0.02210).

It is particularly striking to observe the low p-values obtained for the 15 mg estetrol monohydrate dose by comparison to the elevated p-values obtained for the 10 mg estetrol monohydrate dose.

iii. Frequency Changes in Groups of Responders in Weekly Frequency of Moderate to Severe VMS

VMS frequency was also studied by grouping patients according to their degree of response.

A first grouping of patients showing a response of 50% or more (relative change from baseline) was prepared. According to this analysis, at week 12, the 15 mg estetrol monohydrate daily dose group contains 91.8% of responders, while the placebo group contains 65.5% of responders. The difference between these two groups has a p-value below 0.01, whereas the difference between the 10 mg estetrol monohydrate daily dose group and the placebo group is not statistically significant (p value>0.1).

A second grouping of patients showing a response of 75% or more was prepared. According to this analysis, at week 12, the 15 mg estetrol monohydrate daily dose group contains 77.6% of responders, while the placebo group contains 43.6% of responders. The difference between these two group has a p-value below 0.001, whereas the difference between the 10 mg estetrol monohydrate daily dose group and the placebo group is not statistically significant (p value>0.05).

b. VMS Severity
i. Absolute Change (Mean Change from Baseline) in Weekly Severity of Moderate to Severe VMS

### a) Week-by-Week for Each Group

| | 2.5 mg E4 | 5 mg E4 | 10 mg E4 | 15 mg E4 | |
|---|---|---|---|---|---|
| | monohydrate | monohydrate | monohydrate | monohydrate | Placebo |
| Week | Mean | Mean | Mean | Mean | Mean |
| 0 | 0 | 0 | 0 | 0 | 0 |
| 1 | −0.1542 | −0.098 | −0.2028 | −0.072 | −0.1638 |
| 2 | −0.2112 | −0.1199 | −0.3007 | −0.2853 | −0.2746 |
| 3 | −0.3246 | −0.1574 | −0.3983 | −0.4842 | −0.2676 |
| 4 | −0.3373 | −0.2341 | −0.4842 | −0.5897 | −0.3327 |
| 5 | −0.3984 | −0.1683 | −0.5107 | −0.7432 | −0.4146 |
| 6 | −0.5014 | −0.3212 | −0.6086 | −0.7035 | −0.45 |
| 7 | −0.4562 | −0.2787 | −0.643 | −0.8112 | −0.4645 |
| 8 | −0.5359 | −0.3239 | −0.6644 | −0.8854 | −0.52 |
| 9 | −0.5465 | −0.3249 | −0.6864 | −0.9092 | −0.5522 |
| 10 | −0.6052 | −0.3667 | −0.709 | −0.9628 | −0.5514 |
| 11 | −0.5564 | −0.4161 | −0.666 | −1.0123 | −0.6551 |
| 12 | −0.6279 | −0.4007 | −0.6941 | −1.0425 | −0.6604 |

In order to better analyse the data recorded in this study, treatment groups have been compared using an ANCOVA (analysis of covariance) with respect to the change in severity of moderate to severe VMS from baseline to mild, moderate and severe VMS at week 4 and week 12 for each active treatment versus placebo. For women who experienced 100% VMS relief at week 4 and/or week 12, a value of zero was attributed. The ANCOVA model includes treatment (“trt1”) as a fixed effect and baseline (“base”) as a covariate.

### b) Covariate Significance

The Table below presents a comparison over all treatment groups.

| Analysis Timepoint (N) | Covariate | p_value |
|---|---|---|
| 4 | trt1 | 0.0119 |
| 4 | base | 0.3781 |
| 12 | trt1 | 0.0032 |
| 12 | base | 0.7990 |

c) The Table below presents mean change from baseline by week and treatment:

| Analysis | Planned | LS | Lower 95% | Upper 95% |
|---|---|---|---|---|
| Timepoint | treatment (mg | adjusted | confidence | confidence |
| (N) | E4 monohydrate) | mean | limit | limit |
| 4 | 0 | −0.327801 | −0.470747 | −0.184854 |
| 4 | 2.5 | −0.341092 | −0.486518 | −0.195666 |
| 4 | 5 | −0.238552 | −0.393041 | −0.084063 |
| 4 | 10 | −0.482536 | −0.627769 | −0.337303 |
| 4 | 15 | −0.588755 | −0.739764 | −0.437746 |
| 12 | 0 | −0.658280 | −0.868694 | −0.447865 |
| 12 | 2.5 | −0.629592 | −0.843656 | −0.415528 |
| 12 | 5 | −0.402567 | −0.629971 | −0.175163 |
| 12 | 10 | −0.693404 | −0.907183 | −0.479624 |
| 12 | 15 | −1.042087 | −1.264369 | −0.819805 |

d) The Table below presents differences with placebo by week and treatment

| Analysis | Treatment | | | Lower 95% | Upper 95% | |
|---|---|---|---|---|---|---|
| Timepoint | (mg E4 | | LS adjusted | confidence | confidence | |
| (N) | monohydrate) | Placebo | mean | limit | limit | p_value |
| 4 | 2.5 | 0 | −0.013291 | −0.268767 | 0.242185 | 0.9998 |
| 4 | 5.0 | 0 | 0.089249 | −0.174507 | 0.353004 | 0.8253 |
| 4 | 10.0 | 0 | −0.154735 | −0.409222 | 0.099751 | 0.3767 |
| 4 | 15.0 | 0 | −0.260954 | −0.520749 | −0.001159 | 0.0486 |
| 12 | 2.5 | 0 | 0.028687 | −0.347368 | 0.404743 | 0.9992 |
| 12 | 5.0 | 0 | 0.255713 | −0.132530 | 0.643955 | 0.3062 |
| 12 | 10.0 | 0 | −0.035124 | −0.409723 | 0.339475 | 0.9981 |
| 12 | 15.0 | 0 | −0.383807 | −0.766221 | −0.001394 | 0.0489 |

From this Table it can be seen that the 15 mg estetrol monohydrate daily dose generates a statistically significant difference with placebo at 4 weeks (p-value of 0.0486) and at 12 weeks (p-value of 0.0489). The 15 mg estetrol monohydrate dose thus significantly improves the severity of VMS at weeks 4 and 12 compared with placebo.

For the severity parameter also, the difference between the 10 mg and 15 mg estetrol monohydrate doses is impressive: this is reflected firstly in the mean changes from baseline presented in the Table of section c) above, where for example at 12 weeks the LS adjusted mean for 10 mg is-0.69 (to be compared with −0.66 found for the placebo group), while it is-1.04 for the 15 mg daily dose group. This clear difference is mirrored by a near 8-fold improvement in the p-value at 4 weeks when switching from 10 mg to 15 mg per day, and an over 20-fold improvement in the p-value at 12 weeks when switching from 10 mg to 15 mg per day.

ii. Relative Change (% from Baseline) in Weekly Severity of Moderate to Severe VMS

### a) Week-by-Week for Each Group

| | 2.5 mg E4 | 5 mg E4 | 10 mg E4 | 15 mg E4 | |
|---|---|---|---|---|---|
| | monohydrate | monohydrate | monohydrate | monohydrate | Placebo |
| Week | Mean | Mean | Mean | Mean | Mean |
| 0 | 0% | 0% | 0% | 0% | 0% |
| 1 | −7% | −4% | −8% | −3% | −7% |
| 2 | −9% | −5% | −13% | −12% | −12% |
| 3 | −14% | −7% | −17% | −20% | −11% |
| 4 | −15% | −10% | −20% | −24% | −14% |
| 5 | −17% | −7% | −21% | −31% | −18% |
| 6 | −22% | −14% | −26% | −30% | −19% |
| 7 | −20% | −12% | −27% | −34% | −20% |
| 8 | −24% | −14% | −28% | −37% | −22% |
| 9 | −24% | −14% | −28% | −38% | −23% |
| 10 | −26% | −15% | −29% | −40% | −23% |
| 11 | −25% | −18% | −25% | −42% | −28% |
| 12 | −28% | −17% | −28% | −44% | −27% |

From this table, it can be seen that the 15 mg estetrol monohydrate daily dose resulted in a reduction of over 40% in the severity of moderate to severe VMS when compared to baseline.

### b) Covariate Significance

The Table below presents a comparison over all treatment groups.

| Analysis Timepoint (N) | Covariate | p_value |
|---|---|---|
| 4 | trt1 | 0.0126 |
| 4 | base | 0.6594 |
| 12 | trt1 | 0.0031 |
| 12 | base | 0.1651 |

c) The Table below presents mean relative change from baseline by week and treatment

| Analysis | Planned | LS | Lower 95% | Upper 95% |
|---|---|---|---|---|
| Timepoint | treatment (mg | adjusted | confidence | confidence |
| (N) | E4 monohydrate) | mean | limit | limit |
| 4 | 0 | −0.140987 | −0.200215 | −0.081759 |
| 4 | 2.5 | −0.145345 | −0.205600 | −0.085090 |
| 4 | 5 | −0.095556 | −0.159566 | −0.031546 |
| 4 | 10 | −0.201564 | −0.261739 | −0.141388 |
| 4 | 15 | −0.242707 | −0.305276 | −0.180139 |
| 12 | 0 | −0.279744 | −0.368327 | −0.191161 |
| 12 | 2.5 | −0.275038 | −0.365158 | −0.184919 |
| 12 | 5 | −0.164881 | −0.260616 | −0.069145 |
| 12 | 10 | −0.286159 | −0.376158 | −0.196159 |
| 12 | 15 | −0.437476 | −0.531055 | −0.343897 |

d) The Table below presents differences with placebo by week and treatment

| Analysis | Traetment | | | Lower 95% | Upper 95% | |
|---|---|---|---|---|---|---|
| Timepoint | (mg E4 | | LS adjusted | confidence | confidence | |
| (N) | monohydrate) | Placebo | mean | limit | limit | p_value |
| 4 | 2.5 | 0 | −0.004358 | −0.110211 | 0.101495 | 0.9999 |
| 4 | 5.0 | 0 | 0.045431 | −0.063853 | 0.154714 | 0.7006 |
| 4 | 10.0 | 0 | −0.060577 | −0.166020 | 0.044866 | 0.4281 |
| 4 | 15.0 | 0 | −0.101720 | −0.209363 | 0.005922 | 0.0702 |
| 12 | 2.5 | 0 | 0.004705 | −0.153612 | 0.163022 | 1.0000 |
| 12 | 5.0 | 0 | 0.114863 | −0.048585 | 0.278310 | 0.2522 |
| 12 | 10.0 | 0 | −0.006415 | −0.164119 | 0.151289 | 0.9999 |
| 12 | 15.0 | 0 | −0.157732 | −0.318726 | 0.003261 | 0.0568 |

From this Table it can be seen that the 15 mg estetrol monohydrate daily dose generates a near statistically significant difference with placebo at 12 weeks (p-value of 0.0568). The 15 mg estetrol monohydrate daily dose improves the severity of VMS at weeks 4 and 12 compared with placebo, whereas the 10 mg estetrol monohydrate daily dose here again is hardly distinguishable from placebo, especially so at 12 weeks.

c. Hot Flush Weekly Weighted Score
i. Absolute Change (Mean Change from Baseline) in Weekly Weighted Score

### a) Week-by-Week for Each Group

| | 2.5 mg E4 | 5 mg E4 | 10 mg E4 | 15 mg E4 | |
|---|---|---|---|---|---|
| | monohydrate | monohydrate | monohydrate | monohydrate | Placebo |
| Week | Mean | Mean | Mean | Mean | Mean |
| 0 | 2.4151 | 1.8297 | 2.3773 | 2.219 | 2.0545 |
| 1 | −40.805 | −33.7092 | −31.4591 | −33.1745 | −38.203 |
| 2 | −58.6163 | −49.1319 | −54.0849 | −67.6983 | −64.1272 |
| 3 | −74.8798 | −56.9697 | −73.5189 | −85.1915 | −70.45 |
| 4 | −82.75 | −64.8344 | −87.2673 | −98.8329 | −77.936 |
| 5 | −84.6062 | −71.6181 | −93.4648 | −105.223 | −83.073 |
| 6 | −89.9842 | −88.2337 | −101.646 | −110.268 | −89.6742 |
| 7 | −94.2468 | −88.2989 | −103.024 | −115.263 | −92.9248 |
| 8 | −97.1084 | −89.2039 | −104.835 | −115.656 | −91.6603 |
| 9 | −101.429 | −90.923 | −107.578 | −118.578 | −94.9918 |
| 10 | −100.18 | −91.4798 | −109.514 | −116.853 | −99.4484 |
| 11 | −97.6016 | −95.8365 | −109.532 | −119.365 | −102.771 |
| 12 | −98.5424 | −93.5117 | −111.764 | −121.777 | −104.292 |

### b) Covariate Significance

The Table below presents a comparison over all treatment groups.

| Analysis Timepoint (N) | Covariate | p_value |
|---|---|---|
| 4 | trt1 | 0.0128 |
| 4 | base | <.0001 |
| 12 | trt1 | 0.0107 |
| 12 | base | <.0001 |

c) The Table below presents mean change from baseline by week and treatment:

| Analysis | Planned treatment (mg | LS adjusted | Lower 95% | Upper 95% |
|---|---|---|---|---|
| Timepoint (N) | E4 monohydrate) | mean | confidence limit | confidence limit |
| 4 | 0 | −78.267781 | −93.531339 | −63.004223 |
| 4 | 2.5 | −75.370737 | −91.064629 | −59.676845 |
| 4 | 5 | −66.878234 | −83.400001 | −50.356467 |
| 4 | 10 | −85.563378 | −101.119727 | −70.007030 |
| 4 | 15 | −106.330686 | −122.645517 | −90.015855 |
| 12 | 0 | −104.682568 | −120.535991 | −88.829145 |
| 12 | 2.5 | −89.857250 | −106.157637 | −73.556864 |
| 12 | 5 | −95.915504 | −113.075759 | −78.755249 |
| 12 | 10 | −109.760136 | −125.917663 | −93.602609 |
| 12 | 15 | −130.594733 | −147.540056 | −113.649411 |

d) The Table below presents differences with placebo by week and treatment

| Analysis | Treatment | Placebo | | Lower 95% | Upper 95% | |
|---|---|---|---|---|---|---|
| Timepoint | (mg E4 | (mg E4 | LS adjusted | confidence | confidence | |
| (N) | monohydrate) | monohydrate) | mean | limit | limit | p_value |
| 4 | 2.5 | 0 | 2.897044 | −24.495634 | 30.289722 | 0.9970 |
| 4 | 5.0 | 0 | 11.389547 | −16.739876 | 39.518971 | 0.7205 |
| 4 | 10.0 | 0 | −7.295597 | −34.556270 | 19.965075 | 0.9147 |
| 4 | 15.0 | 0 | −28.062905 | −55.994318 | −0.131492 | 0.0485 |
| 12 | 2.5 | 0 | 14.825318 | −13.625957 | 43.276593 | 0.5176 |
| 12 | 5.0 | 0 | 8.767064 | −20.449428 | 37.983557 | 0.8780 |
| 12 | 10.0 | 0 | −5.077568 | −33.391737 | 23.236601 | 0.9785 |
| 12 | 15.0 | 0 | −25.912165 | −54.922995 | 3.098664 | 0.0951 |

ii. Relative Change (% from Baseline) in Weekly Weighted Score

### a) Week-by-Week for Each Group

| | 2.5 mg E4 | 5 mg E4 | 10 mg E4 | 15 mg E4 | |
|---|---|---|---|---|---|
| | monohydrate | monohydrate | monohydrate | monohydrate | Placebo |
| Week | Mean | Mean | Mean | Mean | Mean |
| 0 | 0% | 0% | 0% | 0% | 0% |
| 1 | −22% | −22% | −20% | −19% | −24% |
| 2 | −32% | −32% | −35% | −43% | −40% |
| 3 | −43% | −37% | −47% | −56% | −43% |
| 4 | −47% | −43% | −54% | −66% | −47% |
| 5 | −48% | −46% | −58% | −70% | −51% |
| 6 | −51% | −56% | −64% | −74% | −55% |
| 7 | −53% | −56% | −64% | −78% | −57% |
| 8 | −55% | −56% | −66% | −78% | −57% |
| 9 | −57% | −58% | −67% | −80% | −58% |
| 10 | −57% | −59% | −68% | −79% | −62% |
| 11 | −56% | −61% | −69% | −80% | −64% |
| 12 | −57% | −60% | −70% | −82% | −65% |

### b) Covariate Significance

The Table below presents a comparison over all treatment groups.

| Analysis Timepoint (N) | Covariate | p_value |
|---|---|---|
| 4 | trt1 | 0.0108 |
| 4 | base | 0.4181 |
| 12 | trt1 | 0.0024 |
| 12 | base | 0.0593 |

c) The Table below presents mean relative change from baseline by week and treatment

| Analysis | Planned | LS | Lower 95% | Upper 95% |
|---|---|---|---|---|
| Timepoint | treatment (mg | adjusted | confidence | confidence |
| (N) | E4 monohydrate) | mean | limit | limit |
| 4 | 0 | −0.474107 | −0.562044 | −0.386170 |
| 4 | 2.5 | −0.473966 | −0.564382 | −0.383549 |
| 4 | 5 | −0.426177 | −0.521362 | −0.330991 |
| 4 | 10 | −0.541886 | −0.631510 | −0.452262 |
| 4 | 15 | −0.650317 | −0.744310 | −0.556323 |
| 12 | 0 | −0.644566 | −0.728624 | −0.560509 |
| 12 | 2.5 | −0.579457 | −0.665884 | −0.493029 |
| 12 | 5 | −0.599251 | −0.690237 | −0.508264 |
| 12 | 10 | −0.699351 | −0.785021 | −0.613681 |
| 12 | 15 | −0.812214 | −0.902061 | −0.722368 |

d) The Table below presents differences with placebo by week and treatment

| Analysis | Treatment | Placebo | | Lower 95% | Upper 95% | |
|---|---|---|---|---|---|---|
| Timepoint | (mg E4 | (mg E4 | LS adjusted | confidence | confidence | |
| (N) | monohydrate) | monohydrate) | mean | limit | limit | p_value |
| 4 | 2.5 | 0 | −0.473966 | −0.564382 | −0.383549 | 1.0000 |
| 4 | 5.0 | 0 | −0.426177 | −0.521362 | −0.330991 | 0.8833 |
| 4 | 10.0 | 0 | −0.541886 | −0.631510 | −0.452262 | 0.6745 |
| 4 | 15.0 | 0 | −0.650317 | −0.744310 | −0.556323 | 0.0267 |
| 12 | 2.5 | 0 | −0.579457 | −0.665884 | −0.493029 | 0.6744 |
| 12 | 5.0 | 0 | −0.599251 | −0.690237 | −0.508264 | 0.8871 |
| 12 | 10.0 | 0 | −0.699351 | −0.785021 | −0.613681 | 0.7861 |
| 12 | 15.0 | 0 | −0.812214 | −0.902061 | −0.722368 | 0.0276 |

From this Table it can be seen that the 15 mg estetrol monohydrate daily dose generates a statistically significant difference with placebo at 4 weeks (p-value of 0.0267) and at 12 weeks (p-value of 0.0276).

It is particularly striking to observe the low p-values obtained for the 15 mg estetrol monohydrate dose by comparison to the elevated p-values obtained for the 10 mg estetrol monohydrate dose.

### B. Vasomotor Parameters for the 10 mg and 15 mg Estetrol Monohydrate Groups by Comparison to the Placebo and Inefficient Doses (2.5 mg and 5 mg Estetrol Monohydrate) Grouped Together

Based on the results observed in section A above, it became apparent that the two lowest doses tested (2.5 mg per day and 5 mg estetrol monohydrate per day) did not show efficacy. A further analysis of the results was thus prepared, where data from these two doses were grouped with the placebo dose and compared to the 10 mg and 15 mg estetrol monohydrate doses.

1. Relative Change (% from Baseline) in Weekly Frequency of Moderate to Severe VMS

### a) Week-by-Week for Each Group

| | 10 mg E4 | | 15 mg E4 | | Placebo with 2.5 and | |
|---|---|---|---|---|---|---|
| | monohydrate | | monohydrate | | 5 mg monohydrate | |
| Week | Mean | SD | Mean | SD | Mean | SD |
| 0 | 0% | 0% | 0% | 0% | 0% | 0% |
| 1 | −21% | 29% | −22% | 33% | −24% | 25% |
| 2 | −37% | 37% | −46% | 34% | −35% | 31% |
| 3 | −48% | 39% | −57% | 34% | −42% | 32% |
| 4 | −55% | 35% | −67% | 30% | −47% | 33% |
| 5 | −60% | 33% | −72% | 28% | −50% | 33% |
| 6 | −65% | 33% | −76% | 26% | −56% | 32% |
| 7 | −66% | 34% | −79% | 25% | −58% | 32% |
| 8 | −68% | 35% | −79% | 26% | −58% | 34% |
| 9 | −69% | 36% | −81% | 24% | −60% | 33% |
| 10 | −70% | 35% | −80% | 27% | −61% | 33% |
| 11 | −70% | 34% | −82% | 25% | −63% | 34% |
| 12 | −72% | 33% | −84% | 23% | −63% | 33% |

### b) Covariate Significance

| Analysis Timepoint (N) | Covariate | p_value |
|---|---|---|
| 4 | trt1 | 0.0039 |
| 4 | base | 0.3935 |
| 4 | SITEPOOL | 0.3308 |
| 12 | trt1 | 0.0017 |
| 12 | base | 0.1244 |
| 12 | SITEPOOL | 0.0957 |

c) Pairwise Comparisons with Placebo (Including Ineffective Doses 2.5 mg and 5 mg Estetrol Monohydrate Grouped Together)

| Analysis | Planned | LS | Lower 95% | Upper 95% |
|---|---|---|---|---|
| Timepoint | treatment (mg | adjusted | confidence | confidence |
| (N) | E4 monohydrate) | mean | limit | limit |
| 4 | 0 | −0.46779276 | −0.521579 | −0.414007 |
| 4 | 10 | −0.54424142 | −0.634204 | −0.454279 |
| 4 | 15 | −0.64752092 | −0.741851 | −0.553191 |
| 12 | 0 | −0.61799510 | −0.669162 | −0.566828 |
| 12 | 10 | −0.70075417 | −0.786337 | −0.615172 |
| 12 | 15 | −0.80058459 | −0.890322 | −0.710847 |

d) Differences with Placebo (Including Ineffective Doses 2.5 mg and 5 mg Estetrol Monohydrate Grouped Together)

| | | Placebo and | | | | |
|---|---|---|---|---|---|---|
| | | ineffective | | | | |
| | | doses 2.5 and | | | | |
| | | 5 mg estetrol | | | | |
| Analysis | Treatment | monohydrate | LS | Lower 95% | Upper 95% | |
| Timepoint | (mg E4 | grouped | adjusted | confidence | confidence | |
| (N) | monohydrate) | together* | mean | limit | limit | p_value |
| 4 | 10.0 | 0 | −0.076449 | −0.193421 | 0.040523 | 0.26148 |
| 4 | 15.0 | 0 | −0.179728 | −0.301777 | −0.057679 | 0.00218 |
| 12 | 10.0 | 0 | −0.082759 | −0.194036 | 0.028518 | 0.17979 |
| 12 | 15.0 | 0 | −0.182589 | −0.298696 | −0.066483 | 0.00099 |

*Placebo and ineffective doses (2.5 and 5 mg estetrol monohydrate) are grouped together and simply referred to as “0”

As already mentioned under Section A) above it is particularly striking to observe the low p-values obtained for the 15 mg estetrol monohydrate daily dose by comparison to the elevated p-values obtained for the 10 mg daily dose, demonstrating the unique relief obtained with the 15 mg estetrol monohydrate daily dose.

2. Relative Change (% from Baseline) in Hot Flush Weekly Weighted Score:

### a) Week-by-Week for Each Group

| | 10 mg E4 | | 15 mg E4 | | Placebo with 2.5 and | |
|---|---|---|---|---|---|---|
| | monohydrate | | monohydrate | | 5 mg monohydrate | |
| Week | Mean | SD | Mean | SD | Mean | SD |
| 0 | 0% | 0% | 0% | 0% | 0% | 0% |
| 1 | −20% | 28% | −22% | 32% | −24% | 25% |
| 2 | −34% | 36% | −45% | 32% | −36% | 30% |
| 3 | −46% | 39% | −56% | 32% | −42% | 31% |
| 4 | −53% | 34% | −66% | 29% | −46% | 32% |
| 5 | −57% | 32% | −70% | 27% | −49% | 33% |
| 6 | −62% | 32% | −72% | 26% | −54% | 32% |
| 7 | −64% | 32% | −75% | 25% | −55% | 31% |
| 8 | −66% | 34% | −76% | 26% | −56% | 32% |
| 9 | −66% | 34% | −78% | 25% | −57% | 31% |
| 10 | −68% | 34% | −77% | 27% | −59% | 32% |
| 11 | −68% | 33% | −79% | 26% | −60% | 33% |
| 12 | −69% | 32% | −81% | 24% | −60% | 32% |

### b) Covariate Significance

| Analysis Timepoint (N) | Covariate | p_value |
|---|---|---|
| 4 | trt1 | 0.0037 |
| 4 | base | 0.5294 |
| 4 | SITEPOOL | 0.3783 |
| 12 | trt1 | 0.0015 |
| 12 | base | 0.0770 |
| 12 | SITEPOOL | 0.0487 |

c) Pairwise Comparisons with Placebo (Including Ineffective Doses 2.5 mg and 5 mg Estetrol Monohydrate)

| Analysis | Planned | LS | Lower 95% | Upper 95% |
|---|---|---|---|---|
| Timepoint | treatment (mg | adjusted | confidence | confidence |
| (N) | E4 monohydrate) | mean | limit | limit |
| 4 | 0 | −0.45362100 | −0.506219 | −0.401023 |
| 4 | 10 | −0.51368261 | −0.601866 | −0.425499 |
| 4 | 15 | −0.63242707 | −0.724866 | −0.539989 |
| 12 | 0 | −0.59132448 | −0.640861 | −0.541788 |
| 12 | 10 | −0.67757463 | −0.760625 | −0.594524 |
| 12 | 15 | −0.76832743 | −0.855386 | −0.681269 |

d) Differences with Placebo (Including Ineffective Doses 2.5 mg and 5 mg Estetrol Monohydrate)

| | | Placebo and | | | | |
|---|---|---|---|---|---|---|
| | | ineffective | | | | |
| | | doses 2.5 and | | | | |
| | | 5 mg estetrol | | | | |
| Analysis | Treatment | monohydrate | LS | Lower 95% | Upper 95% | |
| Timepoint | (mg E4 | grouped | adjusted | confidence | confidence | |
| (N) | monohydrate) | together* | mean | limit | limit | p_value |
| 4 | 10.0 | 0 | −0.060062 | −0.174760 | 0.054636 | 0.41666 |
| 4 | 15.0 | 0 | −0.178806 | −0.298114 | −0.059498 | 0.00178 |
| 12 | 10.0 | 0 | −0.086250 | −0.194272 | 0.021772 | 0.14044 |
| 12 | 15.0 | 0 | −0.177003 | −0.289367 | −0.064639 | 0.00097 |

*Placebo and ineffective doses (2.5 and 5 mg estetrol monohydrate) are grouped together and simply referred to as “0”

As already mentioned under Section A) above, it can be seen that the 15 mg estetrol monohydrate daily dose generates a statistically significant difference with placebo at 4 weeks (p-value of 0.00178) and at 12 weeks (p-value of 0.00097).

Again, it is particularly striking to observe the low p-values obtained for the 15 mg estetrol monohydrate dose by comparison to the elevated p-values obtained for the 10 mg estetrol monohydrate dose.

### C. Menopause Rating Scale

The Menopause Rating Scale (MRS) is a health-related quality of life scale allowing the measure of severity of age-/menopause-related complaints by rating a profile of symptoms (Heinemann et al., 2003, “International versions of the Menopause Rating Scale (MRS)” Health Qual Life Outcomes 1:28; Heinemann et al., 2004, “The Menopause Rating Scale (MRS) scale: A methodological review”. Health Qual Life Outcomes 2:45; Heinemann et al., 2004, “The Menopause Rating Scale (MRS) as outcome measure for hormone treatment? A validation study”. Health Qual Life Outcomes 2:67).

The score increases point by point with increasing severity of subjectively perceived complaints in each one of 11 items (severity expressed in 0 to 4 points in each item). By checking these 5 possible boxes of “severity” for each of the items in the questionnaire, the respondent provides her personal perception. The total MRS score ranges between 0 (asymptomatic) to 44 (highest degree of complaints). The minimal/maximal scores vary between three dimensions depending on the number of complaints allocated to the respective dimension of symptoms (Heinemann et al., 2003, Health Qual Life Outcomes 1:28):
1. psychological symptoms: 0 to 16 scoring points (4 symptoms: depressed, irritable, anxious, exhausted);
2. somato-vegetative symptoms: 0 to 16 points (4 symptoms: sweating/flush, cardiac complaints, sleeping disorders, joint & muscle complaints);
3. urogenital symptoms: 0 to 12 points (3 symptoms: sexual problems, urinary complaints, vaginal dryness).

### Total MRS Score

| | 2.5 mg E4 | | 5 mg E4 | | 10 mg E4 | | 15 mg E4 | | | |
|---|---|---|---|---|---|---|---|---|---|---|
| | mono- | | mono- | | mono- | | mono- | | | |
| | hydrate | | hydrate | | hydrate | | hydrate | | Placebo | |
| Week | Mean | SD | Mean | SD | Mean | SD | Mean | SD | Mean | SD |
| Baseline | 16.5 | 7.2 | 16.5 | 7.3 | 17.6 | 7.6 | 16.4 | 8.1 | 18.2 | 8.9 |
| Week 4 | 10.3 | 5.6 | 10.5 | 6.7 | 11.7 | 6.8 | 8.7 | 6.2 | 12.8 | 8.0 |
| Week 12 | 9.5 | 6.7 | 11.0 | 7.7 | 9.7 | 6.9 | 8.1 | 5.8 | 11.4 | 7.8 |

The Menopause Rating Scale (MRS) points to an overall improvement in quality of life, with the strongest effect for the 15 mg estetrol monohydrate dose. At this dose, a statistically significant effect was observed by comparison with placebo at week 4, with a p-value of 0.0113 and a near statistically significant effect was observed at week 12, with a p-value of 0.0694.

### D. Genito-Urinary Symptoms (GSM)

Change from baseline to week 12 in the following GSM symptoms (VVA subject self-assessment) were recorded:
a) Vaginal dryness (sensation of dryness or burning in the vagina; none=0 mild=1, moderate=2 or severe=3):

| Dose E4 | | | |
|---|---|---|---|
| monohydrate | Baseline | Week 12 | p-value vs |
| (mg) | (mean ± SD) | (mean ± SD) | placebo |
| 2.5 | 1 ± 0.90 | 0.5 ± 0.77 | 0.3345 |
| 5 | 1.3 ± 0.94 | 0.7 ± 0.86 | 0.1202 |
| 10 | 1 ± 0.93 | 0.5 ± 0.75 | 0.0798 |
| 15 | 1.1 ± 1.04 | 0.5 ± 0.68 | 0.0291* |
| Placebo | 1.3 ± 1.10 | 0.9 ± 1.02 | |

*p < 0.05 vs placebo at week 12.

b) Vaginal and/or vulvar irritation/itching (sensation of abnormal irritation or sensitive condition in the vagina; none=0, mild=1, moderate=2 or severe=3):

| Dose E4 | | | |
|---|---|---|---|
| monohydrate | Baseline | Week 12 | p-value vs |
| (mg) | (mean ± SD) | (mean ± SD) | placebo |
| 2.5 | 0.7 ± 0.96 | 0.3 ± 0.60 | 0.1717 |
| 5 | 0.6 ± 0.90 | 0.4 ± 0.65 | 0.9618 |
| 10 | 0.7 ± 0.87 | 0.3 ± 0.64 | 0.2487 |
| 15 | 0.5 ± 0.85 | 0.4 ± 0.70 | 0.931 |
| Placebo | 0.8 ± 0.92 | 0.5 ± 0.77 | |

c) Dysuria (sensation of pain or difficulty in urinating; none=0, mild=1, moderate=2 or severe=3):

| Dose E4 | | | |
|---|---|---|---|
| monohydrate | Baseline | Week 12 | p-value vs |
| (mg) | (mean ± SD) | (mean ± SD) | placebo |
| 2.5 | 0.2 ± 0.56 | 0 ± 0.19 | 0.2942 |
| 5 | 0.2 ± 0.56 | 0 ± 0.20 | 0.3488 |
| 10 | 0.2 ± 0.58 | 0.1 ± 0.23 | 0.3386 |
| 15 | 0.3 ± 0.58 | 0.3 ± 0.61 | 0.643 |
| Placebo | 0.2 ± 0.60 | 0.3 ± 0.55 | |

d) Vaginal pain associated with sexual activity (sensation of pain with sexual intercourse none=0, mild=1, moderate=2 or severe=3):

| Dose E4 | | | |
|---|---|---|---|
| monohydrate | Baseline | Week 12 | p-value vs |
| (mg) | (mean ± SD) | (mean ± SD) | placebo |
| 2.5 | 0.6 ± 0.77 | 0.3 ± 0.73 | 0.0763 |
| 5 | 1 ± 1.07 | 0.5 ± 0.78 | 0.0246* |
| 10 | 0.6 ± 0.77 | 0.2 ± 0.36 | 0.0004** |
| 15 | 0.7 ± 0.90 | 0.3 ± 0.54 | 0.0006** |
| Placebo | 1 ± 1.14 | 0.7 ± 1.01 | |

*p < 0.05 vs placebo at week 12;
**p < 0.001 vs placebo at week 12.

e) Vaginal bleeding associated with sexual activity (loss of blood with sexual intercourse; presence=1 vs. absence=0):

| Dose E4 | Baseline | | Week 12 | | p-value |
|---|---|---|---|---|---|
| mono- | Presence | Absence | Presence | Absence | vs |
| hydrate(mg) | (%) | (%) | (%) | (%) | placebo |
| 2.5 | 0 | 100 | 0 | 100 | 0.9958 |
| 5 | 6.4 | 91.5^{#} | 0 | 100 | 0.903 |
| 10 | 0 | 100 | 0 | 100 | 0.9955 |
| 15 | 0 | 97.9^{#} | 4.2 | 95.8 | 0.9308 |
| Placebo | 3.6 | 92.7^{#} | 3.6 | 96.4 | |

^{#}Some patients had no sexual activity

The evolution of the VVA symptoms points to an overall improvement, with the strongest effect for the 15 mg estetrol monohydrate daily dose. For vaginal pain associated with sexual activity, significant differences with placebo are observed with the doses of 5, 10 and 15 mg estetrol monohydrate daily with p-values of 0.0246, 0.0004 and 0.0006, respectively. Vaginal dryness, however, which is generally considered as the most bothersome symptom, is only significantly improved by the 15 mg estetrol monohydrate daily dose, with a p-value of 0.0291.

### E. Measurements Related to Treatment Side Effects

1. Number of Patients with Biopsies

| | | 2.5 mg | 5 mg | 10 mg | 15 mg |
|---|---|---|---|---|---|
| | | E4 | E4 | E4 | E4 |
| | | mono- | mono- | mono- | mono- |
| Treatment Group | Placebo | hydrate | hydrate | hydrate | hydrate |
| Number of patients | 4 | 4 | 5 | 11 | 9 |
| with biopsies | | | | | |

### 2. Adverse Events (AEs)

| | | 2.5 mg | 5 mg | 10 mg | 15 mg |
|---|---|---|---|---|---|
| | | E4 | E4 | E4 | E4 |
| | | mono- | mono- | mono- | mono- |
| Treatment Group | Placebo | hydrate | hydrate | hydrate | hydrate |
| Count of Treatment | 71 | 61 | 63 | 95 | 82 |
| Emergent AEs (TEAEs) | | | | | |
| Percentage of patients | 9.1 | 7.7 | 10.6 | 7.4 | 6.1 |
| with severe TEAE | | | | | |
| Percentage of patients | 3.6 | 1.9 | 6.4 | 5.6 | 4.1 |
| with TEAE leading to | | | | | |
| Study Discontinuation | | | | | |

It can be seen from the Table above that the patients in the 15 mg estetrol monohydrate group present less TEAEs than patients in the 10 mg estetrol monohydrate group. In the 10 mg estetrol monohydrate group of patients who had AEs, the average was 3.2 AEs per patient. By comparison, in the 15 mg estetrol monohydrate group, patients who had AEs had on average 2.6 AEs. Globally, those data show that the 15 mg estetrol monohydrate daily dose provides a significant relief of VMS without generating additional AEs for the patients. In addition, there were less requirements for biopsies in the 15 mg per day group than in the 10 mg estetrol monohydrate per day group.

This is confirmed by the following statistical analysis. Using a Poisson regression model with a random effect for the patient and treatment group as a covariate to model the count of TEAEs in the different treatment groups, it can be shown that there is no statistical difference between the treatment groups (p value of 0.099). Second, a chi-square test was used to assess if the prevalence of patients reporting TEAEs in each treatment group was similar. No statistical difference was found between the treatment groups (p value of 0.575).

### 3. Patients Leaving the Study

| | | 2.5 mg | 5 mg | 10 mg | 15 mg |
|---|---|---|---|---|---|
| | | E4 | E4 | E4 | E4 |
| | | mono- | mono- | mono- | mono- |
| Treatment Group | Placebo | hydrate | hydrate | hydrate | hydrate |
| Number of patients | 14 | 9 | 11 | 15 | 8 |
| leaving the study | | | | | |

### F. Measurements Performed at 15 mg and 20 mg Estetrol Monohydrate Daily Doses

In order to better assess the potential for increasing the daily dose beyond the minimum effective dose of 15 mg estetrol monohydrate daily, a number of parameters were followed in a study where estetrol monohydrate was administered at the increased dose of 20 mg per day.

1. Triglycerides Level (mmol/L)

| | | | End Of Treatment * | |
|---|---|---|---|---|
| | | | | Actual Change |
| Treatment | Parameter | Baseline | Actual Value | from Baseline |
| E4 15 mg | Mean | 1.32 | 1.61 | 0.20 |
| monohydrate | SD | 0.64 | 0.94 | 0.82 |
| | Median | 1.21 | 1.40 | 0.15 |
| E4 20 mg | Mean | 1.43 | 1.53 | 0.10 |
| monohydrate | SD | 0.47 | 0.63 | 0.44 |
| | Median | 1.40 | 1.40 | 0.10 |

* End Of Treatment was after 28 days for the 20 mg dose and after 12 weeks for the 15 mg dose.

2. Glucose Level (mmol/L)

| | | | End Of Treatment * | |
|---|---|---|---|---|
| | | | | Actual Change |
| Treatment | Parameter | Baseline | Actual Value | from Baseline |
| E4 15 mg | Mean | 4.77 | 4.79 | 0.02 |
| monohydrate | SD | 0.37 | 0.58 | 0.56 |
| | Median | 4.72 | 4.77 | 0.05 |
| E4 20 mg | Mean | 5.53 | 5.44 | −0.10 |
| monohydrate | SD | 0.54 | 0.50 | 0.22 |
| | Median | 5.60 | 5.60 | −0.15 |

* End Of Treatment was after 28 days for the 20 mg dose and after 12 weeks for the 15 mg dose.

3. Cholesterol Level (mmol/L)

| | | | End Of Treatment * | |
|---|---|---|---|---|
| | | | | Actual Change |
| Treatment | Parameter | Baseline | Actual Value | from Baseline |
| E4 15 mg | Mean | 5.51 | 5.63 | 0.12 |
| monohydrate | SD | 0.83 | 0.97 | 0.73 |
| | Median | 5.62 | 5.71 | 0.08 |
| E4 20 mg | Mean | 6.27 | 6.09 | −0.18 |
| monohydrate | SD | 0.90 | 0.96 | 0.65 |
| | Median | 6.25 | 6.10 | −0.20 |

* End Of Treatment was after 28 days for the 20 mg dose and after 12 weeks for the 15 mg dose.

4. HDL-Cholesterol Level (mmol/L)

| | | | End Of Treatment * | |
|---|---|---|---|---|
| | | | | Actual Change |
| Treatment | Parameter | Baseline | Actual Value | from Baseline |
| E4 15 mg | Mean | 1.73 | 1.89 | 0.16 |
| monohydrate | SD | 0.46 | 0.41 | 0.24 |
| | Median | 1.66 | 1.81 | 0.17 |
| E4 20 mg | Mean | 1.68 | 1.97 | 0.29 |
| monohydrate | SD | 0.37 | 0.36 | 0.14 |
| | Median | 1.60 | 1.90 | 0.35 |

* End Of Treatment was after 28 days for the 20 mg dose and after 12 weeks for the 15 mg dose.

5. LDL-Cholesterol Level (mmol/L)

| | | | End Of Treatment * | |
|---|---|---|---|---|
| | | | | Actual Change |
| Treatment | Parameter | Baseline | Actual Value | from Baseline |
| E4 15 mg | Mean | 3.05 | 3.14 | 0.09 |
| monohydrate | SD | 0.85 | 0.95 | 0.45 |
| | Median | 3.22 | 3.25 | 0.10 |
| E4 20 mg | Mean | 3.94 | 3.43 | −0.51 |
| monohydrate | SD | 0.98 | 1.05 | 0.56 |
| | Median | 4.00 | 3.45 | −0.65 |

* End Of Treatment was after 28 days for the 20 mg dose and after 12 weeks for the 15 mg dose.

From the above 5 Tables it can be observed that these lipid parameters and glucose level do not behave significantly differently when a 20 mg estetrol monohydrate daily dose is used in place of a 15 mg estetrol monohydrate daily dose.

### 6. C-Terminal Telopeptide (CTX-1) (Ng/L)

| | | | End Of Treatment * | |
|---|---|---|---|---|
| | | | | Actual Change |
| Treatment | Parameter | Baseline | Actual Value | from Baseline |
| E4 15 mg | Mean | 416.0 | 320.9 | −95.0 |
| monohydrate | SD | 286.16 | 359.65 | 442.18 |
| | Median | 339.0 | 197.5 | −142.5 |
| E4 20 mg | Mean | 422.50 | 274.9 | −147.60 |
| monohydrate | SD | 134.8 | 95.3 | 70.6 |
| | Median | 416.5 | 267.50 | −138.0 |

* End Of Treatment was after 28 days for the 20 mg dose and after 12 weeks for the 15 mg dose.

CTX-1 is a specific marker of bone resorption.

In the Table above it can been seen that the 15 mg estetrol monohydrate daily dose leads to a small decrease of bone resorption and this effect is more pronounced with the 20 mg estetrol monohydrate daily dose already after 28 days of treatment.

### G. Measurements Performed at 15 mg and 30 mg Estetrol Monohydrate Daily Doses

The following treatments were administered to healthy women (between 15-50 years inclusive) according to the randomization code.
Placebo (n=16);
Group 15 mg: a single oral dose of 15 mg Estetrol monohydrate/3 mg Drospirenone (n=10) followed, after a washout of 14 days, by multiple oral doses of 15 mg Estetrol monohydrate/3 mg Drospirenone (n=10) once daily for 14 days;
Group 30 mg: a single oral dose of 30 mg Estetrol monohydrate/6 mg Drospirenone (n=10), followed, after a washout of 14 days, by multiple oral doses of 30 mg Estetrol monohydrate/6 mg Drospirenone (n=10) once daily for 14 days.

Adverse events were recorded from first admission until completion of the follow-up visit (between 37 to 42 days after first day of treatment).

| | | 15 mg E4 | 30 mg E4 |
|---|---|---|---|
| Treatment Emergent AEs (TEAEs) by | | mono- | mono- |
| relationship to study drug | Placebo | hydrate | hydrate |
| Total | 75% | 80% | 70% |
| Related | 31% | 50% | 50% |
| Unrelated | 75% | 50% | 50% |

% = number of subjects reporting one or more AE as percentage of the total number of subjects in the corresponding treatment group

Overall, single dose administration and 14-day once daily administration of oral Estetrol monohydrate/Drospirenone doses in the range of 15 mg Estetrol monohydrate/3 mg Drospirenone to 30 mg Estetrol monohydrate/6 mg Drospirenone were safe and well-tolerated by the healthy female subjects in this study. With increasing single and multiple Estetrol monohydrate/Drospirenone doses (dose was doubled), no increase in either percentage of subjects reporting TEAEs or the number of TEAEs was observed.

It is thus reasonable to envision a hormone replacement therapy for alleviating menopause-associated symptoms which uses a daily dose of estetrol monohydrate of between 15 mg, the minimal effective dose, and 20 mg, or even 25 mg which will allow a better benefit-to-risk profile to be obtained. Increasing the estetrol monohydrate dose beyond the minimal effective dose of 15 mg per day will indeed offer even better efficacy in VMS relief and in parameters such as bone resorption, while maintaining an excellent safety profile (including, but not limited to, Adverse Events, as presented under Sections E) 2) and G) above, and particularly lipid parameters and glucose level presented under Section F) 1) to 5) above). Increasing the estetrol monohydrate dose beyond the minimal effective dose of 15 mg per day will also permit a faster onset of relief to be obtained when patients initiate hormone replacement therapy.

### Example 3. Comparison Between 15 Mg and 20 Mg Estetrol Monohydrate

A further clinical study was designed to evaluate the effect of 15 or 20 mg estetrol (E4) monohydrate, or placebo on the severity and frequency of vasomotor symptoms (VMS) and the safety of E4 20 mg. The inclusion and exclusion criteria as described for Example 2 were maintained for the present Example (i.e. Example 3).

End points:

### Primary Outcome Measures

1. Mean change in weekly frequency of moderate to severe vasomotor symptoms (VMS) from Baseline to Week 4 (Efficacy Study Part) [Time Frame: Baseline and Week 4]:
   The weekly frequency of moderate to severe VMS at Baseline and Week 4 is defined as the total number (sum) of all recorded moderate to severe VMS experienced during the last 7 consecutive days prior randomization for Baseline and at Week 4. Mean change=mean weekly frequency at Week 4-mean weekly frequency at Baseline.
2. Mean change in weekly frequency of moderate to severe vasomotor symptoms (VMS) from Baseline to Week 12 (Efficacy Study Part) [Time Frame: Baseline and Week 12]
   The weekly frequency of moderate to severe VMS at Baseline and Week 12 is defined as the total number (sum) of all recorded moderate to severe VMS experienced during the last 7 consecutive days prior randomization for Baseline and at Week 12. Mean change=mean weekly frequency at Week 12-mean weekly frequency at Baseline.
3. Mean change in severity of moderate to severe vasomotor symptoms (VMS) from Baseline to Week 4 (Efficacy Study Part) [Time Frame: Baseline and Week 4]
   The severity score is derived as follows: mild=1, moderate=2, and severe=3. The mean severity score of VMS at Baseline and Week 4 is defined as the arithmetic mean of the daily severity score values of moderate and severe VMS observed from the last 7 days prior randomization for Baseline and of moderate and severe VMS observed at Week 4. Baseline and Week 4 severity score=[(2×number of moderate VMS)+(3×number of severe VMS)]/(total number of moderate+severe VMS). Mean change=mean severity score at Week 4-mean severity score at Baseline.
4. Mean change in severity of moderate to severe vasomotor symptoms (VMS) from Baseline to Week 12 (Efficacy Study Part) [Time Frame: Baseline and Week 12]
   The severity score is derived as follows: mild=1, moderate=2, and severe=3. The mean severity score of VMS at Baseline and Week 12 is defined as the arithmetic mean of the daily severity score values of moderate and severe VMS observed from the last 7 days prior randomization for Baseline and of moderate and severe VMS observed at Week 12. Baseline and Week 12 severity score=[(2×number of moderate VMS)+(3×number of severe VMS)]/(total number of moderate+severe VMS). Mean change=mean severity score at Week 12-mean severity score at Baseline.
5. Incidence of endometrial hyperplasia with up to 12 months of treatment based on endometrial biopsies (Endometrial and General Safety Study Part) [Time Frame: Screening and Week 53]
   Endometrial biopsies will be centrally evaluated by three independent expert pathologists from different institutions, blinded to treatment group and to each other's readings. The concurrence of two of the three pathologists will be accepted as the final diagnosis. If there is no agreement among the three pathologists, the most severe pathologic diagnosis, i.e., atypical hyperplasia>complex hyperplasia>simple hyperplasia>benign endometrium, will be used as the final diagnosis.

### Secondary Outcome Measures

1. Mean change from Baseline to Week n in the weekly frequency and severity of moderate to severe vasomotor symptoms (VMS) (Efficacy Study Part) [Time Frame: Baseline and Week n] (wherein n is an integer selected from 1 to 12).
   The weekly frequency of moderate to severe VMS at Baseline and Week n is defined as the total number (sum) of all recorded moderate to severe VMS experienced during the last 7 consecutive days prior randomization for Baseline and at Week n. Mean change=mean weekly frequency at Week n-mean weekly frequency at Baseline. The severity score is derived as follows: mild=1, moderate=2, and severe=3. The mean severity score of VMS at Baseline and Week n is defined as the arithmetic mean of the daily severity score values of moderate and severe VMS observed from the last 7 days prior randomization for Baseline and of moderate and severe VMS observed at Week n. Baseline and Week n severity score=[(2×number of moderate VMS)+(3×number of severe VMS)]/(total number of moderate+severe VMS). Mean change=mean severity score at Week n-mean severity score at Baseline.
2. Mean change from Baseline to Week n in the weekly frequency and severity of mild, moderate and severe vasomotor symptoms (VMS) (Efficacy Study Part) [Time Frame: Baseline and Week n] (wherein n is an integer selected from 1 to 12).
   The severity score is derived as follows: mild=1, moderate=2, and severe=3. The mean severity score of VMS at Baseline and Week n is defined as the arithmetic mean of the daily severity score values of mild, moderate and severe VMS observed from the last 7 days prior randomization for Baseline and of mild, moderate and severe VMS observed at Week n. Baseline and Week n severity score=[(1×number of mild VMS)+(2×number of moderate VMS)+(3×number of severe VMS)]/(total number of mild+moderate+severe VMS). Mean change=mean severity score at Week n-mean severity score at Baseline
3. Percentage of participants with 50% reduction from Baseline in the weekly frequency of moderate to severe vasomotor symptoms (VMS) at Week 1, 2, 3, 4, 5, 6, 7, 8, 9, 10, 11, and 12 (Efficacy Study Part) [Time Frame: Baseline, Week 1, 2, 3, 4, 5, 6, 7, 8, 9, 10, 11, and 12]
   The weekly frequency of moderate to severe VMS at Baseline and Week X is defined as the total number (sum) of all recorded moderate to severe VMS experienced during the last 7 consecutive days prior randomization (Baseline) and day [(X-1)*7+1] to day X*7 (Week X).
4. Percentage of participants with 50% reduction from Baseline in the weekly frequency of mild, moderate, and severe vasomotor symptoms (VMS) at Week 1, 2, 3, 4, 5, 6, 7, 8, 9, 10, 11, and 12 (Efficacy Study Part) [Time Frame: Baseline, Week 1, 2, 3, 4, 5, 6, 7, 8, 9, 10, 11, and 12]
   The weekly frequency of moderate to severe VMS at Baseline and Week X is defined as the total number (sum) of all recorded moderate to severe VMS experienced during the last 7 consecutive days prior randomization (Baseline) and day [(X-1)*7+1] to day X*7 (Week X).
5. Percentage of participants with 75% reduction from Baseline in the weekly frequency of moderate to severe VMS at Week 1, 2, 3, 4, 5, 6, 7, 8, 9, 10, 11, and 12 (Efficacy Study Part) [Time Frame: Baseline, Week 1, 2, 3, 4, 5, 6, 7, 8, 9, 10, 11, and 12]
   The weekly frequency of moderate to severe VMS at Baseline and Week X is defined as the total number (sum) of all recorded moderate to severe VMS experienced during the last 7 consecutive days prior randomization (Baseline) and day [(X-1)*7+1] to day X*7 (Week X).
6. Percentage of participants with 75% reduction from Baseline in the weekly frequency of mild, moderate, and severe VMS at Week 1, 2, 3, 4, 5, 6, 7, 8, 9, 10, 11, and 12 (Efficacy Study Part) [Time Frame: Baseline, Week 1, 2, 3, 4, 5, 6, 7, 8, 9, 10, 11, and 12]
   The weekly frequency of moderate to severe VMS at Baseline and Week X is defined as the total number (sum) of all recorded moderate to severe VMS experienced during the last 7 consecutive days prior randomization (Baseline) and day [(X-1)*7+1] to day X*7 (Week X).
7. Percentage of participants with a clinically important difference (CID) compared to Baseline in the weekly frequency of moderate to severe VMS at Week 4 and 12 using the Clinical Global Impression (CGI) questionnaire (Efficacy Study Part) [Time Frame: Week 4 and 12]
   The CGI score is a seven point scale in which subjects will be asked to rate the total improvement, whether or not in her judgment it was due entirely to drug treatment, compared to her condition at admission to the study. Scale: Very much improved, Much improved, Minimally improved, No change, Minimally worse, Much worse, Very much worse.
8. Change from Baseline to Week 12 in VVA symptoms (Efficacy Study Part) [Time Frame: Baseline and Week 12]
   GSM will be assessed by the subjects using the VVA self-assessment questionnaire. The following GSM symptoms will be assessed:
      Vaginal dryness
      Vaginal and/or vulvar irritation/itching
      Dysuria
      Vaginal pain associated with sexual activity
      Vaginal bleeding associated with sexual activity
   All GSM symptoms except vaginal bleeding associated with sexual activity will be graded by the participants using the following scale: [0] none, [1] mild, [2] moderate, or [3] severe. Vaginal bleeding associated with sexual activity is documented using 2 categories: [0] absent or [1] present. A negative change from baseline score indicates improvement in symptoms.
9. Change from Baseline to Week 12 in the VVA symptom that is initially identified by the participant as being the most bothersome using the VVA questionnaire at baseline (Efficacy Study Part) [Time Frame: Baseline and Week 12]
   GSM will be assessed by the subjects using the VVA self-assessment questionnaire. The following GSM symptoms will be assessed:
      Vaginal dryness
      Vaginal and/or vulvar irritation/itching
      Dysuria
      Vaginal pain associated with sexual activity
      Vaginal bleeding associated with sexual activity
   All GSM symptoms except vaginal bleeding associated with sexual activity were graded by the participants using the following scale: [0] none, [1] mild, [2] moderate, or [3] severe. Vaginal bleeding associated with sexual activity was documented using 2 categories: [0] absent or [1] present. A negative change from baseline score indicates improvement in symptoms. At baseline the participant will be asked which of the above mentioned symptoms she identifies as being the most bothersome.
10. Change from Baseline to Week 12 in plasma concentration of triglycerides, plasma concentration of low-density lipoprotein (LDL)-cholesterol, plasma concentration of total cholesterol, in the total cholesterol/high density cholesterol (HDL) cholesterol ratio, in the HDL-cholesterol ratio, in plasma concentration of lipoprotein (a), in fasting glycaemia, in plasma concentration of insulin, in plasma concentration of glycated hemoglobin, in Homeostasis model-assessment-estimated insulin resistance (HOMA-IR), in health-related quality of life assessment (HRQOL) using the menopause-specific Quality of Life (MENQOL) questionnaire (Efficacy Study Part) [Time Frame: Baseline and Week 12]
   The MENQOL is self-administered questionnaire which will assess changes in quality of life over a one-month period. It is composed of 29 questions indicating if subject experienced the problem (Yes/No) and if Yes, rating scale ranged from 0=Not bothered at all to 6=Extremely bothered. For analysis, the original scores will be converted to the analysis score ranging from 1-8 where No=1, 0=2, 1=3 . . . and 6=8. The scale contains four domains: vasomotor, psychosocial, physical and sexual. Each domain is scored separately. Vasomotor domain score is mean of =Q1, Q2, Q3, with 1 being “not at all bothered” and 8 being “extremely bothered”.
11. Total score in treatment satisfaction using the Clinical Global Impression (CGI) questionnaire (Efficacy Study Part) [Time Frame: Weeks 4 and 12]
   The CGI score is a seven point scale in which subjects will be asked to rate the total improvement, whether or not in her judgment it was due entirely to drug treatment, compared to her condition at admission to the study. Scale: Very much improved, Much improved, Minimally improved, No change, Minimally worse, Much worse, Very much worse.
12. Number of participants with treatment-emergent adverse events (TEAEs) (Efficacy Study Part) [Time Frame: From baseline to Follow-up visit (up to Week 16)]
   TEAEs are those adverse events occurring from time point of first ingestion of investigational product until last visit or any event already present that worsens in either intensity or frequency following exposure to the treatment.
13. Number of participants with changes in physical and gynaecological examination results (Efficacy Study Part) [Time Frame: Screening and Week 13]
   Physical examination will include an examination of general appearance, head, eyes, ears, nose, throat, skin, neck, lungs, breast, lymph nodes, abdomen, and the cardiovascular musculoskeletal and neurological systems. Gynaecological examination will include a manual pelvic examination.
14. Number of participants with changes in vital sign results (Efficacy Study Part) [Time Frame: From screening to Week 13]
   Vital signs will include height, body weight, body mass index, sitting systolic and diastolic blood pressures, and heart rate.
15. Number of participants with changes in electrocardiogram (ECG) results (Efficacy Study Part) [Time Frame: Screening and Week 13]
   The ECG interpretation scheme will include the analysis of the morphology, rhythm, conduction, ST segment, PR, QRS, QT and corrected QT (QTc) intervals, T waves, U waves and the presence or absence of any pathological changes.
16. Number of participants with changes in breast examination results (Efficacy Study Part) [Time Frame: Screening and Week 13]
   Number of participants with changes in routine clinical laboratory test results (Efficacy Study Part) [Time Frame: Screening, Baseline and Week 13]. Routine laboratory tests include hematology and chemistry.
17. Change from baseline to each measured time point in endometrial thickness (Efficacy Study Part) [Time Frame: Screening, Week 13, Week 16]
   Endometrial thickness will be assessed by transvaginal ultrasound (TVUS). Baseline: data will be recorded at Screening.
18. Frequency of subjects in the different endometrial categories according to Blaustein's pathology (Efficacy Study Part) [Time Frame: Screening and Week 13]
   Endometrial biopsies will be centrally evaluated by three independent expert pathologists from different institutions, blinded to treatment group and to each other's readings. The concurrence of two of the three pathologists will be accepted as the final diagnosis. If there is no agreement among the three pathologists, the most severe pathologic diagnosis, i.e., atypical hyperplasia>complex hyperplasia>simple hyperplasia>benign endometrium, will be used as the final diagnosis.
19. Number of participants with vaginal bleeding and/or spotting during each 28-day cycle of treatment with E4 (Efficacy Study Part) [Time Frame: From Baseline up to Follow-up (Week 16)]
   Vaginal bleeding will be daily recorded by the participant on the diary. Absence or occurrence of vaginal bleeding/spotting will be assessed using the scale below: 0=Absence of vaginal bleeding or spotting; 1=Spotting: evidence of minimal blood loss requiring none or at most one pad, tampon or panty liner per day; 2=Bleeding: evidence of blood loss requiring more than one pad, tampon or panty liner per day.
20. Number of days with bleeding and/or spotting during each 28-day cycle of treatment (Efficacy Study Part) [Time Frame: From Baseline up to Follow-up (Week 16)]
   Vaginal bleeding will be daily recorded by the participant on the diary. Absence or occurrence of vaginal bleeding/spotting will be assessed using the scale below: 0=Absence of vaginal bleeding or spotting; 1=Spotting: evidence of minimal blood loss requiring none or at most one pad, tampon or panty liner per day; 2=Bleeding: evidence of blood loss requiring more than one pad, tampon or panty liner per day.
21. Number of participants with amenorrhea (absence of any bleeding or spotting) during each 28-day cycle of treatment with E4 (Efficacy Study Part) [Time Frame: From Baseline up to Follow-up (Week 16)]
   Vaginal bleeding will be daily recorded by the participant on the diary. Absence or occurrence of vaginal bleeding/spotting will be assessed using the scale below: 0=Absence of vaginal bleeding or spotting; 1=Spotting: evidence of minimal blood loss requiring none or at most one pad, tampon or panty liner per day; 2=Bleeding: evidence of blood loss requiring more than one pad, tampon or panty liner per day.
22. Cumulative rates of amenorrhea (Efficacy Study Part) [Time Frame: From Baseline up to Follow-up (Week 16)]
   The rate of amenorrhea is defined as the percentage of women who reported consecutive cycles of amenorrhea for a given cycle of time.
23. Number of participants with treatment-emergent adverse events (TEAEs) (Endometrial and General Safety Part) [Time Frame: From baseline to Week 53]
   TEAEs are those adverse events occurring from time point of first ingestion of investigational product until last visit or any event already present that worsens in either intensity or frequency following exposure to the treatment.
24. Number of participants with changes in physical and gynaecological examination results, changes in vital sign results, changes in breast examination results, changes in electrocardiogram (ECG) results, changes in mammography results, changes in routine clinical laboratory test results (Endometrial and General Safety Part) [Time Frame: Screening and Week 53]
   Physical examination will include an examination of general appearance, head, eyes, ears, nose, throat, skin, neck, lungs, breast, lymph nodes, abdomen, and the cardiovascular musculoskeletal and neurological systems. Gynaecological examination will include a manual pelvic examination. Vital signs will include height, body weight, body mass index, sitting systolic and diastolic blood pressures, and heart rate. The ECG interpretation scheme will include the analysis of the morphology, rhythm, conduction, ST segment, PR, QRS, QT and corrected QT (QTc) intervals, T waves, U waves and the presence or absence of any pathological changes. Routine laboratory tests include haematology and chemistry.
25. Number of women with vaginal bleeding and/or spotting during each 28-day cycle of treatment with E4 (Endometrial and General Safety Part) [Time Frame: From Baseline to Week 53]
   Vaginal bleeding will be daily recorded by the participant on the diary. Absence or occurrence of vaginal bleeding/spotting will be assessed using the scale below: 0=Absence of vaginal bleeding or spotting; 1=Spotting: evidence of minimal blood loss requiring none or at most one pad, tampon or panty liner per day; 2=Bleeding: evidence of blood loss requiring more than one pad, tampon or panty liner per day.
26. Number of days with bleeding and/or spotting during each 28-day cycle of treatment (Endometrial and General Safety Part) [Time Frame: From Baseline to Week 53]
   Vaginal bleeding will be daily recorded by the participant on the diary. Absence or occurrence of vaginal bleeding/spotting will be assessed using the scale below: 0=Absence of vaginal bleeding or spotting; 1=Spotting: evidence of minimal blood loss requiring none or at most one pad, tampon or panty liner per day; 2=Bleeding: evidence of blood loss requiring more than one pad, tampon or panty liner per day.
27. Number of participants with amenorrhea (absence of any bleeding or spotting) during each 28-day cycle of treatment with E4 (Endometrial and General Safety Part) [Time Frame: From Baseline to Week 53]
   Vaginal bleeding will be daily recorded by the participant on the diary. Absence or occurrence of vaginal bleeding/spotting will be assessed using the scale below: 0=Absence of vaginal bleeding or spotting; 1=Spotting: evidence of minimal blood loss requiring none or at most one pad, tampon or panty liner per day; 2=Bleeding: evidence of blood loss requiring more than one pad, tampon or panty liner per day.
28. Cumulative rates of amenorrhea (Endometrial and General Safety Part) [Time Frame: From Baseline to Week 53]
   The rate of amenorrhea is defined as the percentage of women who reported consecutive cycles of amenorrhea for a given cycle of time.
29. Change from Baseline to Weeks 12 and 52 in health-related quality of life assessment (HRQOL) using the menopause-specific Quality of Life (MENQOL) questionnaire (Endometrial and General Safety Part) [Time Frame: Baseline and Weeks 12 and 52]
   The MENQOL is self-administered questionnaire which will assess changes in quality of life over a one-month period. It is composed of 29 questions indicating if subject experienced the problem (Yes/No) and if Yes, rating scale ranged from 0=Not bothered at all to 6=Extremely bothered. For analysis, the original scores were converted to the analysis score ranging from 1-8 where No=1, 0=2, 1=3 . . . and 6=8. The scale contains four domains: vasomotor, psychosocial, physical and sexual. Each domain is scored separately. Vasomotor domain score is mean of =Q1, Q2, Q3, with 1 being “not at all bothered” and 8 being “extremely bothered”.
30. Total score in treatment satisfaction assessed after 4, 12 and 52 weeks of treatment using the Clinical Global Impression (CGI) questionnaire (Endometrial and General Safety Part) [Time Frame: Weeks 4, 12, and 52]
   The CGI score is a seven point scale in which subjects will be asked to rate the total improvement, whether or not in her judgment it was due entirely to drug treatment, compared to her condition at admission to the study. Scale: Very much improved, Much improved, Minimally improved, No change, Minimally worse, Much worse, Very much worse.
31. Change from Baseline to Weeks 12 and 52 in plasma concentration of triglycerides, in plasma concentration of high-density lipoprotein (HDL)-cholesterol, in plasma concentration of low-density lipoprotein (LDL)-cholesterol, in plasma concentration of total cholesterol, in the total cholesterol/high density cholesterol (HDL) cholesterol ratio, in plasma concentration of lipoprotein (a), in fasting glycaemia, in plasma concentration of insulin, in plasma concentration of glycated haemoglobin, in Homeostasis model-assessment-estimated insulin resistance (HOMA-IR), in endometrial thickness (Endometrial and General Safety Part) [Time Frame: Baseline and Weeks 12 and 52]
   Endometrial thickness will be assessed by transvaginal ultrasound (TVUS).
32. Frequency of subjects in the different endometrial categories according to Blaustein's pathology (Endometrial and General Safety Study Part) [Time Frame: Screening and Week 53]
   Endometrial biopsies will be centrally evaluated by three independent expert pathologists from different institutions, blinded to treatment group and to each other's readings. The concurrence of two of the three pathologists will be accepted as the final diagnosis. If there is no agreement among the three pathologists, the most severe pathologic diagnosis, i.e., atypical hyperplasia>complex hyperplasia>simple hyperplasia>benign endometrium, will be used as the final diagnosis.

### Results

### 1. VMS

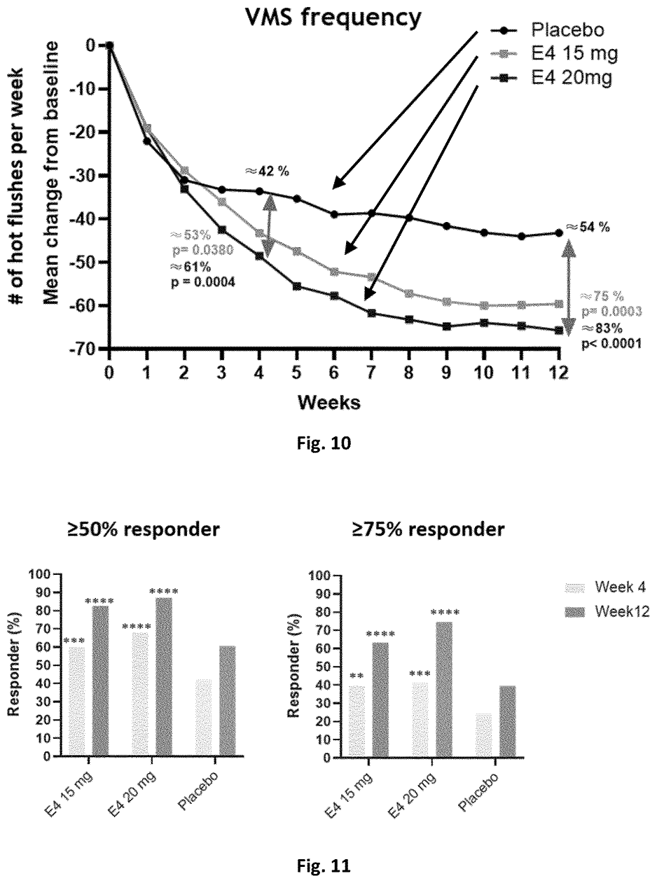

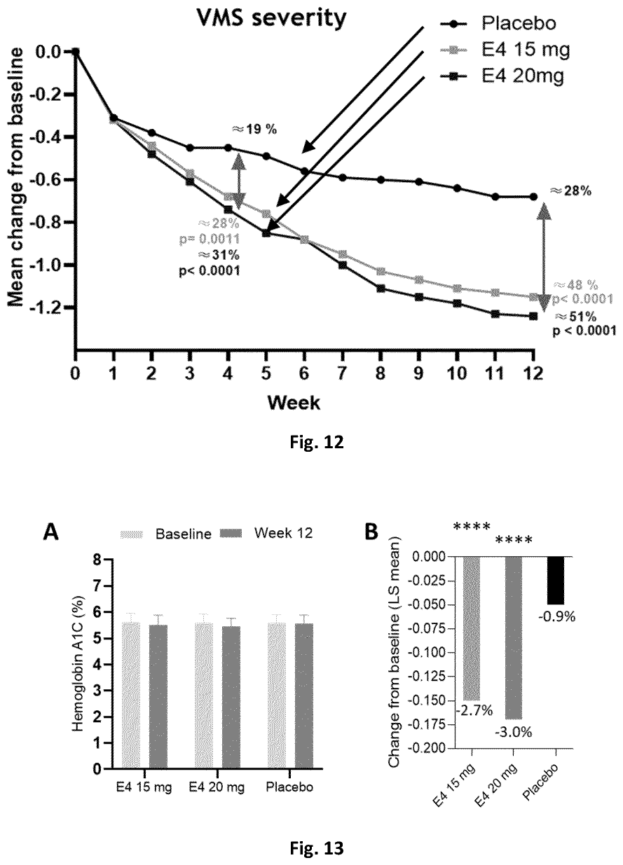

From the data, it could be deducted that estetrol significantly decreased the frequency of moderate to severe VMS (FIG. 10). In addition, estetrol treatment resulted in a higher number of women that experienced a reduced VMS frequency than placebo (FIG. 11). Finally, estetrol monohydrate significantly decreased the severity of moderate to severe VMS (FIG. 12).

### 2. Endometrial Thickness

No significant difference in endometrial thickness could be observed upon comparison of the 15 mg estetrol monohydrate with the 20 mg estetrol monohydrate treatment group, as substantiated by an adjusted p-value of 0.8560 by ANCOVA analysis. Both doses resulted in an equal increase as expected from an estrogen treatment.

### 3. MENQOL

The total MENQOL scores of the subjects treated with estetrol monohydrate were significantly improved when compared to placebo treatment. This improvement stems from several distinct parameters that contribute to the total MENQOL score, including but not limited to improvements in terms of reported muscle aching, sleeping difficulties, anxiety and/or nervousness, impatience, degree of dissatisfaction relating to personal life, and the psychosocial domain score.

| | | Treatment | | Adj |
|---|---|---|---|---|
| Parameter | Treatment 1 | 2 | Estimate | P-value |
| Total MENQOL Score | E4 monoh. 15 | Placebo | −0.7068 | <.0001 |
| | mg | | | |
| | E4 monoh. 20 | Placebo | −0.7155 | <.0001 |
| | mg | | | |

Physical Domain

| | | | | |
|---|---|---|---|---|
| Aching in muscles | E4 monoh. 15 | Placebo | −0.1858 | 0.6146 |
| | mg | | | |
| | E4 monoh. 20 | Placebo | −0.5576 | 0.0130 |
| | mg | | | |

Psychosocial Domain

| | | | | |
|---|---|---|---|---|
| Psychosocial Domain | E4 monoh. 15 | Placebo | −0.5211 | 0.0035 |
| Score | mg | | | |
| | E4 monoh. 20 | Placebo | −0.6056 | 0.0005 |
| | mg | | | |
| Difficulty sleeping | E4 monoh. 15 | Placebo | −0.7011 | 0.0050 |
| | mg | | | |
| | E4 monoh. 20 | Placebo | −0.7258 | 0.0031 |
| | mg | | | |
| Anxious or Nervous | E4 monoh. 15 | Placebo | −0.6100 | 0.0054 |
| | mg | | | |
| | E4 monoh. 20 | Placebo | −0.8197 | 0.0002 |
| | mg | | | |
| Being Impatient with | E4 monoh. 15 | Placebo | −0.4911 | 0.0643 |
| Others | mg | | | |
| | E4 monoh. 20 | Placebo | −0.5656 | 0.0241 |
| | mg | | | |
| Dissatisfied Personal | E4 monoh. 15 | Placebo | −0.8163 | 0.0181 |
| Life | mg | | | |
| | E4 monoh. 20 | Placebo | −1.2719 | 0.0003 |
| | mg | | | |

### 4. Diabetes

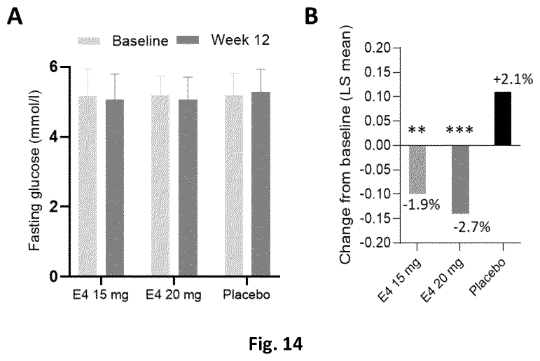

Treatment of subjects with 15 mg or 20 mg estetrol monohydrate positively impacted glucose metabolism in terms of reduced haemoglobin A1C (FIG. 13) and fasting glucose (FIG. 14).

### Haemoglobin A1C:

| | Baseline | | | Week 12 | | |
|---|---|---|---|---|---|---|
| | mean | SD | n | mean | SD | n |
| E4 15 mg | 5.625 | 0.3342 | 211 | 5.504 | 0.3869 | 151 |
| monohydrate | | | | | | |
| E4 20 mg | 5.605 | 0.3244 | 213 | 5.458 | 0.3137 | 153 |
| monohydrate | | | | | | |
| Placebo | 5.599 | 0.3189 | 214 | 5.567 | 0.323 | 163 |

P-value compared to placebo

| 15 mg E4 | 20 mg E4 |
|---|---|
| monohydrate | monohydrate |
| <.0001 | <.0001 |

### Fasting Glucose:

| | Baseline | | | Week 12 | | |
|---|---|---|---|---|---|---|
| | mean | SD | n | mean | SD | n |
| E4 15 mg | 5.156 | 0.7805 | 212 | 5.057 | 0.7342 | 157 |
| monohydrate | | | | | | |
| E4 20 mg | 5.177 | 0.5648 | 210 | 5.057 | 0.6474 | 151 |
| monohydrate | | | | | | |
| Placebo | 5.193 | 0.614 | 212 | 5.285 | 0.6424 | 155 |

P-value compared to placebo

| 15 mg E4 | 20 mg E4 |
|---|---|
| monohydrate | monohydrate |
| 0.0020 | 0.0002 |

### Example 4. a Dose-Finding Study to Select the Daily Oral Dose of Estetrol (E4) for the Treatment of Vasomotor Symptoms in Post-Menopausal Women with Mild, Moderate, or Severe Hepatic Impairment

The study described in Example 2 is carried out again whilst also including women with mild, moderate, or severe hepatic impairment. In women with mild, moderate, or severe hepatic impairment and preferably in women with mild or moderate hepatic impairment, there will be no statistically significant differences in efficacy and safety compared to women with normal hepatic function. In particular, it is emphasized that the number of biopsies with both 15 and 20 mg estetrol monohydrate dosage in the groups with and without hepatic impairment will not differ. Particularly, there will be no difference in the number of biopsies in women with mild or moderate hepatic impairment when compared to the number of biopsies in women with normal hepatic function when the distinct groups are normalized for sample size. In conclusion, the metabolism of estetrol monohydrate in women with hepatic impairment is not affected in such a way that dose adjustment is necessary. Instead, women with hepatic impairment can simply receive the same dose as women with normal hepatic function.

1-16. (canceled)

17. A method for alleviating estrogen deficiency symptoms in a hepatically impaired subject, comprising orally administering a composition formulated as an oral dosage unit to a hepatically impaired subject, wherein said composition comprises an estetrol component and is administered at a daily dose equivalent to from about 15 mg to about 25 mg of estetrol.

18. The method of claim 17, wherein the hepatically impaired subject is selected from a subject exhibiting mild hepatic impairment (Child-Pugh score of 5 or 6 points), moderate hepatic impairment (Child-Pugh score of 7 to 9 points), or severe hepatic impairment (Child-Pugh score of 10 to 15 points), as classified by the Child-Pugh scoring system.

19. The method of claim 17, wherein the subject is a female menopausal, perimenopausal, or post-menopausal subject, and the estrogen deficiency symptoms are menopause-associated symptoms.

20. The method of claim 17, wherein the method is effective for alleviating estrogen deficiency symptoms as assessed by one or more selected from reducing vasomotor symptoms (VMS) frequency, reducing VMS severity, reducing hot flush weekly weighted score, reducing dryness of the vagina, reducing dyspareunia, improving Quality of Life according to the Menopause Rating Scale (MRS) questionnaire, and improving Quality of Life according to the Menopause-specific Quality of Life (MENQOL) questionnaire.

21. The method of claim 20, wherein the VMS comprise one or more selected from hot flashes, sweating attacks, night sweats, chills, increased perspiration, and palpitations.

22. The method of claim 19, wherein the method is effective for alleviating one or more emotional aspects of menopausal transition selected from depression, irritability, mood changes, insomnia, sleep disturbance, anxiety, and nervous tension.

23. The method of claim 19, wherein the method is effective for alleviating one or more physiological aspects of menopausal transition selected from joint pain, loss of bone density, urinary tract infections, urinary incontinence, dryness of the vagina, uterine prolapse, changes in skin texture, weight gain, dyspareunia, cardiovascular diseases, and diabetes.

24. The method of claim 17, wherein a single administration of the oral dosage unit provides the hepatically impaired subject with a pharmacokinetic profile having a geometric mean (GM) Cₘₐₓ for estetrol (E4) which is less than 2-fold of a corresponding GM Cₘₐₓ in a subject with normal hepatic function.

25. The method of claim 24, wherein a single administration of the oral dosage unit provides the hepatically impaired subject with a pharmacokinetic profile having a geometric mean (GM) Cₘₐₓ for estetrol (E4) which is 1.7-fold or less of a corresponding GM Cₘₐₓ in a subject with normal hepatic function.

26. The method of claim 17, wherein a single administration of the oral dosage unit provides the hepatically impaired subject with a pharmacokinetic profile having a GM AUC_{inf} for estetrol (E4) which is less than 2-fold of a corresponding GM AUC_{inf} in a subject with normal hepatic function.

27. The method of claim 26, wherein a single administration of the oral dosage unit provides the hepatically impaired subject with a pharmacokinetic profile having a GM AUC_{inf} for estetrol (E4) which is 1.1-fold or less of a corresponding GM AUC_{inf} in a subject with normal hepatic function.

28. The method of claim 17, wherein a single administration of the oral dosage unit provides the hepatically impaired subject with a pharmacokinetic profile having a GM AUC_{inf} for E4-3 glucuronide which does not significantly differ from a corresponding GM AUC_{inf} of E4-3 glucuronide in a subject with a normal hepatic function.

29. The method of claim 17, wherein a single administration of the oral dosage unit provides the hepatically impaired subject with a pharmacokinetic profile having a GM T½ for estetrol (E4) which is similar to a corresponding GM T½ in a subject with normal hepatic function.

30. The method of claim 17, wherein the number, frequency, and/or severity of adverse effects do not differ between a population of hepatically impaired subjects and a population of subjects with normal hepatic function.

31. The method of claim 17, wherein the estetrol component is estetrol monohydrate.

32. The method of claim 31, wherein the oral dosage unit is administered at a daily dose selected from about 15 mg estetrol monohydrate and about 20 mg estetrol monohydrate.

33. The method of claim 17, wherein the composition further comprises a progestogenic component, optionally selected from progesterone, drospirenone, norethisterone, norethisteron-acetate (NETA), dydrogesterone, levonorgestrel (LNG), etonogestrel, norgestrel, nomegestrol, nomegestrol-acetate (NOMAC), trimegestone, nestorone, dydrogesterone, gestodene, desogestrel, norgestimate, cyproterone acetate, dienogest, and chlormadinone, or wherein the composition further comprises bazedoxifene.

34. The method of claim 33, wherein the progestogenic component is drospirenone, and is administered at a daily dose of from about 1 mg to about 4 mg of drospirenone.

35. The method of claim 34, wherein the drospirenone is administered at a daily dose of about 3 mg drospirenone.

36. The method of claim 17, wherein the oral dosage unit is formulated as a daily dosage unit.
